SEQUENCE LISTING (81 sequences)

SEQ ID NO: 1 - DNA, 900 bp, Homo sapiens
  CDS (1)..(900)
atgctagagtctcacctcattatctattttcttcttgcagtgatacaatttcttcttgggattttcacaaatggcatcattgtggtggtgaatggcattgacttgatcaagcacagaaaaatggctccgctggatctccttctttcttgtctggcagtttctagaatttttctgcagttgttcatcttctacgttaatgtgattgttatcttcttcatagaattcatcatgtgttctgcgaattgtgcaattctcttatttataaatgaattggaactttggcttgccacatggctcggcgttttctattgtgccaaggttgccagcgtccgtcacccactcttcatctggttgaagatgaggatatccaagctggtcccatggatgatcctggggtctctgctatatgtatctatgatttgtgttttccatagcaaatatgcagggtttatggtcccatacttcctaaggaaatttttctcccaaaatgccacaattcaaaaagaagatacactggctatacagattttctcttttgttgctgagttctcagtgccattgcttatcttcctttttgctgttttgctcttgattttctctctggggaggcacacccggcaaatgagaaacacagtggccggcagcagggttcctggcaggggtgcacccatcagcgcgttgctgtctatcctgtccttcctgatcctctacttctcccactgcatgataaaagtttttctctcttctctaaagtttcacatcagaaggttcatctttctgttcttcatccttgtgattggtatatacccttctggacactctctcatcttaattttaggaaatcctaaattgaaacaaaatgcaaaaaagttcctcctccacagtaagtgctgtcagtga

SEQ ID NO: 2 - protein, 299 aa, Homo sapiens
MLESHLIIYFLLAVIQFLLGIFTNGIIVVVNGIDLIKHRKMAPLDLLLSCLAVSRIFLQLFIFYVNVIVIFFIEFIMCSANCAILLFINELELWLATWLGVFYCAKVASVRHPLFIWLKMRISKLVPWMILGSLLYVSMICVFHSKYAGFMVPYFLRKFFSQNATIQKEDTLAIQIFSFVAEFSVPLLIFLFAVLLLIFSLGRHTRQMRNTVAGSRVPGRGAPISALLSILSFLILYFSHCMIKVFLSSLKFHIRRFIFLFFILVIGIYPSGHSLILILGNPKLKQNAKKFLLHSKCCQ

SEQ ID NO: 3 - DNA, 951 bp, Homo sapiens
  CDS (1)..(951)
atgatgggactcaccgagggggtgttcctgattctgtctggcactcagttcacactgggaattctggtcaattgtttcattgagttggtcaatggtagcagctggttcaagaccaagagaatgtctttgtctgacttcatcatcaccaccctggcactcttgaggatcattctgctgtgtattatcttgactgatagttttttaatagaattctctcccaacacacatgattcagggataataatgcaaattattgatgtttcctggacatttacaaaccatctgagcatttggcttgccacctgtcttggtgtcctctactgcctgaaaatcgccagtttctctcaccccacattcctctggctcaagtggagagtttctagggtgatggtatggatgctgttgggtgcactgctcttatcctgtggtagtaccgcatctctgatcaatgagtttaagctctattctgtctttaggggaattgaggccaccaggaatgtgactgaacacttcagaaagaagaggagtgagtattatctgatccatgttcttgggactctgtggtacctgcctcccttaattgtgtccctggcctcctactctttgctcatcttctccctggggaggcacacacggcagatgctgcaaaatgggacaagctccagagatccaaccactgaggcccacaagagggccatcagaatcatcctttccttcttctttctcttcttactttactttcttgctttcttaattgcatcatttggtaatttcctaccaaaaaccaagatggctaagatgattggcgaagtaatgacaatgttttatcctgctggccactcatttattctcattctggggaacagtaagctgaagcagacatttgtagtgatgctccggtgtgagtctggtcatctgaagcctggatccaagggacccattttctcttag

SEQ ID NO: 4 - protein, 316 aa, Homo sapiens
MMGLTEGVFLILSGTQFTLGILVNCFIELVNGSSWFKTKRMSLSDFIITTLALLRIILLCIILTDSFLIEFSPNTHDSGIIMQIIDVSWTFTNHLSIWLATCLGVLYCLKIASFSHPTFLWLKWRVSRVMVWMLLGALLLSCGSTASLINEFKLYSVFRGIEATRNVTEHFRKKRSEYYLIHVLGTLWYLPPLIVSLASYSLLIFSLGRHTRQMLQNGTSSRDPTTEAHKRAIRIILSFFFLFLLYFLAFLIASFGNFLPKTKMAKMIGEVMTMFYPAGHSFILILGNSKLKQTFVVMLRCESGHLKPGSKGPIFS

SEQ ID NO: 5 - DNA, 900 bp, Homo sapiens
  CDS (1)..(900)
atgcttcggttattctatttctctgctattattgcctcagttattttaaattttgtaggaatcattatgaatctgtttattacagtggtcaattgcaaaacttgggtcaaaagccatagaatctcctcttctgataggattctgttcagcctgggcatcaccaggtttcttatgctgggactatttctggtgaacaccatctacttcgtctcttcaaatacggaaaggtcagtctacctgtctgctttttttgtgttgtgtttcatgtttttggactcgagcagtgtctggtttgtgaccttgctcaatatcttgtactgtgtgaagattactaacttccaacactcagtgtttctcctgctgaagcggaatatctccccaaagatccccaggctgctgctggcctgtgtgctgatttctgctttcaccacttgcctgtacatcacgcttagccaggcatcaccttttcctgaacttgtgactacgagaaataacacatcatttaatatcagtgagggcatcttgtctttagtggtttctttggtcttgagctcatctctccagttcatcattaatgtgacttctgcttccttgctaatacactccttgaggagacatatacagaagatgcagaaaaatgccactggtttctggaatccccagacggaagctcatgtaggtgctatgaagctgatggtctatttcctcatcctctacattccatattcagttgctaccctggtccagtatctccccttttatgcagggatggatatggggaccaaatccatttgtctgatttttgccaccctttactctccaggacattctgttctcattattatcacacatcctaaactgaaaacaacagcaaagaagattctttgtttcaaaaaatag

SEQ ID NO: 6 - protein, 299 aa, Homo sapiens
MLRLFYFSAIIASVILNFVGIIMNLFITVVNCKTWVKSHRISSSDRILFSLGITRFLMLGLFLVNTIYFVSSNTERSVYLSAFFVLCFMFLDSSSVWFVTLLNILYCVKITNFQHSVFLLLKRNISPKIPRLLLACVLISAFTTCLYITLSQASPFPELVTTRNNTSFNISEGILSLVVSLVLSSSLQFIINVTSASLLIHSLRRHIQKMQKNATGFWNPQTEAHVGAMKLMVYFLILYIPYSVATLVQYLPFYAGMDMGTKSICLIFATLYSPGHSVLIIITHPKLKTTAKKILCFKK

SEQ ID NO: 7 - DNA, 900 bp, Homo sapiens
  CDS (1)..(900)
atgctgagcgctggcctaggactgctgatgctggtggcagtggttgaatttctcatcggtttaattggaaatggaagcctggtggtctggagttttagagaatggatcagaaaattcaactggtcctcatataacctcattatcctgggcctggctggctgccgatttctcctgcagtggctgatcattttggacttaagcttgtttccacttttccagagcagccgttggcttcgctatcttagtatcttctgggtcctggtaagccaggccagcttatggtttgccaccttcctcagtgtcttctattgcaagaagatcacgaccttcgatcgcccggcctacttgtggctgaagcagagggcctataacctgagtctctggtgccttctgggctactttataatcaatttgttacttacagtccaaattggcttaacattctatcatcctccccaaggaaacagcagcattcggtatccctttgaaagctggcagtacctgtatgcatttcagctcaattcaggaagttatttgcctttagtggtgtttcttgtttcctctgggatgctgattgtctctttgtatacacaccacaagaagatgaaggtccattcagctggtaggagggatgtccgggccaaggctcacatcactgcgctgaagtccttgggctgcttcctcttacttcacctggtttatatcatggccagccccttctccatcacctccaagacttatcctcctgatctcaccagtgtcttcatctgggagacactcatggcagcctatccttctcttcattctctcatattgatcatggggattcctagggtgaagcagacttgtcagaagatcctgtggaagacagtgtgtgctcggagatgctggggcccatga

SEQ ID NO: 8 - protein, 299 aa, Homo sapiens
MLSAGLGLLMLVAVVEFLIGLIGNGSLVVWSFREWIRKFNWSSYNLIILGLAGCRFLLQWLIILDLSLFPLFQSSRWLRYLSIFWVLVSQASLWFATFLSVFYCKKITTFDRPAYLWLKQRAYNLSLWCLLGYFIINLLLTVQIGLTFYHPPQGNSSIRYPFESWQYLYAFQLNSGSYLPLVVFLVSSGMLIVSLYTHHKKMKVHSAGRRDVRAKAHITALKSLGCFLLLHLVYIMASPFSITSKTYPPDLTSVFIWETLMAAYPSLHSLILIMGIPRVKQTCQKILWKTVCARRCWGP

SEQ ID NO: 9 - DNA, 957 bp, Homo sapiens
  CDS (1)..(957)
atggcagataaagtgcagactactttattgttcttagcagttggagagttttcagtggggatcttagggaatgcattcattggattggtaaactgcatggactgggtcaagaagaggaaaattgcctccattgatttaatcctcacaagtctggccatatccagaatttgtctattgtgcgtaatactattagattgttttatattggtgctatatccagatgtctatgccactggtaaagaaatgagaatcattgacttcttctggacactaaccaatcatttaagtatctggtttgcaacctgcctcagcatttactatttcttcaagataggtaatttctttcacccacttttcctctggatgaagtggagaattgacagggtgatttcctggattctactggggtgcgtggttctctctgtgtttattagccttccagccactgagaatttgaacgctgatttcaggttttgtgtgaaggcaaagaggaaaacaaacttaacttggagttgcagagtaaataaaactcaacatgcttctaccaagttatttctcaacctggcaacgctgctccccttttgtgtgtgcctaatgtcctttttcctcttgatcctctccctgcggagacatatcaggcgaatgcagctcagtgccacagggtgcagagaccccagcacagaagcccatgtgagagccctgaaagctgtcatttccttccttctcctctttattgcctactatttgtcctttctcattgccacctccagctactttatgccagagacggaattagctgtgatttttggtgagtccatagctctaatctacccctcaagtcattcatttatcctaatactggggaacaataaattaagacatgcatctctaaaggtgatttggaaagtaatgtctattctaaaaggaagaaaattccaacaacataaacaaatctga

SEQ ID NO: 10 - protein, 318 aa, Homo sapiens
MADKVQTTLLFLAVGEFSVGILGNAFIGLVNCMDWVKKRKIASIDLILTSLAISRICLLCVILLDCFILVLYPDVYATGKEMRIIDFFWTLTNHLSIWFATCLSIYYFFKIGNFFHPLFLWMKWRIDRVISWILLGCVVLSVFISLPATENLNADFRFCVKAKRKTNLTWSCRVNKTQHASTKLFLNLATLLPFCVCLMSFFLLILSLRRHIRRMQLSATGCRDPSTEAHVRALKAVISFLLLFIAYYLSFLIATSSYFMPETELAVIFGESIALIYPSSHSFILILGNNKLRHASLKVIWKVMSILKGRKFQQHKQI

SEQ ID NO: 11 - DNA, 930 bp, Homo sapiens
  CDS (1)..(930)
atgttcagtcctgcagataacatctttataatcctaataactggagaattcatactaggaatattggggaatggatacattgcactagtcaactggattgactggattaagaagaaaaagatttccacagttgactacatccttaccaatttagttatcgccagaatttgtttgatcagtgtaatggttgtaaatggcattgtaatagtactgaacccagatgtttatacaaaaaataaacaacagatagtcatttttaccttctggacatttgccaactacttaaatatgtggattaccacctgccttaatgtcttctattttctgaagatagccagttcctctcatccactttttctctggctgaagtggaaaattgatatggtggtgcactggatcctgctgggatgctttgccatttccttgttggtcagccttatagcagcaatagtactgagttgtgattataggtttcatgcaattgccaaacataaaagaaacattactgaaatgttccatgtgagtaaaataccatactttgaacccttgactctctttaacctgtttgcaattgtcccatttattgtgtcactgatatcatttttccttttagtaagatctttatggagacataccaagcaaataaaactctatgctaccggcagtagagaccccagcacagaagttcatgtgagagccattaaaactatgacttcatttatcttcttttttttcctatactatatttcttctattttgatgacctttagctatcttatgacaaaatacaagttagctgtggagtttggagagattgcagcaattctctaccccttgggtcactcacttattttaattgttttaaataataaactgaggcagacatttgtcagaatgctgacatgtagaaaaattgcctgcatgatatga

SEQ ID NO: 12 - protein, 309 aa, Homo sapiens
MFSPADNIFIILITGEFILGILGNGYIALVNWIDWIKKKKISTVDYILTNLVIARICLISVMVVNGIVIVLNPDVYTKNKQQIVIFTFWTFANYLNMWITTCLNVFYFLKIASSSHPLFLWLKWKIDMVVHWILLGCFAISLLVSLIAAIVLSCDYRFHAIAKHKRNITEMFHVSKIPYFEPLTLFNLFAIVPFIVSLISFFLLVRSLWRHTKQIKLYATGSRDPSTEVHVRAIKTMTSFIFFFFLYYISSILMTFSYLMTKYKLAVEFGEIAAILYPLGHSLILIVLNNKLRQTFVRMLTCRKIACMI

SEQ ID NO: 13 - DNA, 939 bp, Homo sapiens
  CDS (1)..(939)
atgccaagtgcaatagaggcaatatatattattttaattgctggtgaattgaccatagggatttggggaaatggattcattgtactagttaactgcattgactggctcaaaagaagagatatttccttgattgacatcatcctgatcagcttggccatctccagaatctgtctgctgtgtgtaatatcattagatggcttctttatgctgctctttccaggtacatatggcaatagcgtgctagtaagcattgtgaatgttgtctggacatttgccaataattcaagtctctggtttacttcttgcctcagtatcttctatttactcaagatagccaatatatcgcacccatttttcttctggctgaagctaaagatcaacaaggtcatgcttgcgattcttctggggtcctttcttatctctttaattattagtgttccaaagaatgatgatatgtggtatcaccttttcaaagtcagtcatgaagaaaacattacttggaaattcaaagtgagtaaaattccaggtactttcaaacagttaaccctgaacctgggggtgatggttccctttatcctttgcctgatctcatttttcttgttacttttctccctagttagacacaccaagcagattcgactgcatgctacagggttcagagaccccagtacagaggcccacatgagggccataaaggcagtgatcatctttctgctcctcctcatcgtgtactacccagtctttcttgttatgacctctagcgctctgattcctcagggaaaattagtgttgatgattggtgacatagtaactgtcattttcccatcaagccattcattcattctaattatgggaaatagcaagttgagggaagcttttctgaagatgttaagatttgtgaagtgtttccttagaagaagaaagccttttgttccatag

SEQ ID NO: 14 - protein, 312 aa, Homo sapiens
MPSAIEAIYIILIAGELTIGIWGNGFIVLVNCIDWLKRRDISLIDIILISLAISRICLLCVISLDGFFMLLFPGTYGNSVLVSIVNVVWTFANNSSLWFTSCLSIFYLLKIANISHPFFFWLKLKINKVMLAILLGSFLISLIISVPKNDDMWYHLFKVSHEENITWKFKVSKIPGTFKQLTLNLGVMVPFILCLISFFLLLFSLVRHTKQIRLHATGFRDPSTEAHMRAIKAVIIFLLLLIVYYPVFLVMTSSALIPQGKLVLMIGDIVTVIFPSSHSFILIMGNSKLREAFLKMLRFVKCFLRRRKPFVP

SEQ ID NO: 15 - DNA, 924 bp, Homo sapiens
  CDS (1)..(924)
atgctacgtgtagtggaaggcatcttcatttttgttgtagttagtgagtcagtgtttggggttttggggaatggatttattggacttgtaaactgcattgactgtgccaagaataagttatctacgattggctttattctcaccggcttagctatttcaagaatttttctgatatggataataattacagatggatttatacagatattctctccaaatatatatgcctccggtaacctaattgaatatattagttacttttgggtaattggtaatcaatcaagtatgtggtttgccaccagcctcagcatcttctatttcctgaagatagcaaatttttccaactacatatttctctggttgaagagcagaacaaatatggttcttcccttcatgatagtattcttacttatttcatcgttacttaattttgcatacattgcgaagattcttaatgattataaaacgaagaatgacacagtctgggatctcaacatgtataaaagtgaatactttattaaacagattttgctaaatctgggagtcattttcttctttacactatccctaattacatgtatttttttaatcatttccctttggagacacaacaggcagatgcaatcgaatgtgacaggattgagagactccaacacagaagctcatgtgaaggcaatgaaagttttgatatctttcatcatcctctttatcttgtattttataggcatggccatagaaatatcatgttttactgtgcgagaaaacaaactgctgcttatgtttggaatgacaaccacagccatctatccctggggtcactcatttatcttaattctaggaaacagcaagctaaagcaagcctctttgagggtactgcagcaattgaagtgctgtgagaaaaggaaaaatctcagagtcacatag

SEQ ID NO: 16 - protein, 307 aa, Homo sapiens
MLRVVEGIFIFVVVSESVFGVLGNGFIGLVNCIDCAKNKLSTIGFILTGLAISRIFLIWIIITDGFIQIFSPNIYASGNLIEYISYFWVIGNQSSMWFATSLSIFYFLKIANFSNYIFLWLKSRTNMVLPFMIVFLLISSLLNFAYIAKILNDYKTKNDTVWDLNMYKSEYFIKQILLNLGVIFFFTLSLITCIFLIISLWRHNRQMQSNVTGLRDSNTEAHVKAMKVLISFIILFILYFIGMAIEISCFTVRENKLLLMFGMTTTAIYPWGHSFILILGNSKLKQASLRVLQQLKCCEKRKNLRVT

SEQ ID NO: 17 - DNA, 912 bp, Homo sapiens
  CDS (1)..(912)
atggaaagtgccctgccgagtatcttcactcttgtaataattgcagaattcataattgggaatttgagcaatggatttatagtactgatcaactgcattgactgggtcagtaaaagagagctgtcctcagtcgataaactcctcattatcttggcaatctccagaattgggctgatctgggaaatattagtaagttggtttttagctctgcattatctagccatatttgtgtctggaacaggattaagaattatgatttttagctggatagtttctaatcacttcaatctctggcttgctacaatcttcagcatcttttatttgctcaaaatagcgagtttctctagccctgcttttctctatttgaagtggagagtaaacaaagtgattctgatgatactgctaggaaccttggtcttcttatttttaaatctgatacaaataaacatgcatataaaagactggctggaccgatatgaaagaaacacaacttggaatttcagtatgagtgactttgaaacattttcagtgtcggtcaaattcactatgactatgttcagtctaacaccatttactgtggccttcatctcttttctcctgttaattttctccctgcagaaacatctccagaaaatgcaactcaattacaaaggacacagagaccccaggaccaaggtccatacaaatgccttgaaaattgtgatctcattccttttattctatgctagtttctttctatgtgttctcatatcatggatttctgagctgtatcagaacacagtgatctacatgctttgtgagacgattggagtcttctctccttcaagccactcctttcttctgattctaggaaacgctaagttaagacaggcctttcttttggtggcagctaaggtatgggctaaacgatga

SEQ ID NO: 18 - protein, 303 aa, Homo sapiens
MESALPSIFTLVIIAEFIIGNLSNGFIVLINCIDWVSKRELSSVDKLLIILAISRIGLIWEILVSWFLALHYLAIFVSGTGLRIMIFSWIVSNHFNLWLATIFSIFYLLKIASFSSPAFLYLKWRVNKVILMILLGTLVFLFLNLIQINMHIKDWLDRYERNTTWNFSMSDFETFSVSVKFTMTMFSLTPFTVAFISFLLLIFSLQKHLQKMQLNYKGHRDPRTKVHTNALKIVISFLLFYASFFLCVLISWISELYQNTVIYMLCETIGVFSPSSHSFLLILGNAKLRQAFLLVAAKVWAKR

SEQ ID NO: 19 - DNA, 954 bp, Homo sapiens
  CDS (1)..(954)
atgggtggtgtcataaagagcatatttacattcgttttaattgtggaatttataattggaaatttaggaaatagtttcatagcactggtgaactgtattgactgggtcaagggaagaaagatctcttcggttgatcggatcctcactgctttggcaatctctcgaattagcctggtttggttaatattcggaagctggtgtgtgtctgtgtttttcccagctttatttgccactgaaaaaatgttcagaatgcttactaatatctggacagtgatcaatcattttagtgtctggttagctacaggcctcggtactttttattttctcaagatagccaatttttctaactctatttttctctacctaaagtggagggttaaaaaggtggttttggtgctgcttcttgtgacttcggtcttcttgtttttaaatattgcactgataaacatccatataaatgccagtatcaatggatacagaagaaacaagacttgcagttctgattcaagtaactttacacgattttccagtcttattgtattaaccagcactgtgttcattttcataccctttactttgtccctggcaatgtttcttctcctcatcttctccatgtggaaacatcgcaagaagatgcagcacactgtcaaaatatccggagacgccagcaccaaagcccacagaggagttaaaagtgtgatcactttcttcctactctatgccattttctctctgtcttttttcatatcagtttggacctctgaaaggttggaggaaaatctaattattctttcccaggtgatgggaatggcttatccttcatgtcactcatgtgttctgattcttggaaacaagaagctgagacaggcctctctgtcagtgctactgtggctgaggtacatgttcaaagatggggagccctcaggtcacaaagaatttagagaatcatcttga

SEQ ID NO: 20 - protein, 317 aa, Homo sapiens
MGGVIKSIFTFVLIVEFIIGNLGNSFIALVNCIDWVKGRKISSVDRILTALAISRISLVWLIFGSWCVSVFFPALFATEKMFRMLTNIWTVINHFSVWLATGLGTFYFLKIANFSNSIFLYLKWRVKKVVLVLLLVTSVFLFLNIALINIHINASINGYRRNKTCSSDSSNFTRFSSLIVLTSTVFIFIPFTLSLAMFLLLIFSMWKHRKKMQHTVKISGDASTKAHRGVKSVITFFLLYAIFSLSFFISVWTSERLEENLIILSQVMGMAYPSCHSCVLILGNKKLRQASLSVLLWLRYMFKDGEPSGHKEFRESS

SEQ ID NO: 21 - DNA, 876 bp, Homo sapiens
  CDS (1)..(876)
atgatacccatccaactcactgtcttcttcatgatcatctatgtgcttgagtccttgacaattattgtgcagagcagcctaattgttgcagtgctgggcagagaatggctgcaagtcagaaggctgatgcctgtggacatgattctcatcagcctgggcatctctcgcttctgtctacagtgggcatcaatgctgaacaatttttgctcctattttaatttgaattatgtactttgcaacttaacaatcacctgggaattttttaatatccttacattctggttaaacagcttgcttaccgtgttctactgcatcaaggtctcttctttcacccatcacatctttctctggctgaggtggagaattttgaggttgtttccctggatattactgggttctctgatgattacttgtgtaacaatcatcccttcagctattgggaattacattcaaattcagttactcaccatggagcatctaccaagaaacagcactgtaactgacaaacttgaaaattttcatcagtatcagttccaggctcatacagttgcattggttattcctttcatcctgttcctggcctccaccatctttctcatggcatcactgaccaagcagatacaacatcatagcactggtcactgcaatccaagcatgaaagcgcgcttcactgccctgaggtcccttgccgtcttatttattgtgtttacctcttactttctaaccatactcatcaccattataggtactctatttgataagagatgttggttatgggtctgggaagcttttgtctatgctttcatcttaatgcattccacttcactgatgctgagcagccctacgttgaaaaggattctaaagggaaagtgctag

SEQ ID NO: 22 - protein, 291 aa, Homo sapiens
MIPIQLTVFFMIIYVLESLTIIVQSSLIVAVLGREWLQVRRLMPVDMILISLGISRFCLQWASMLNNFCSYFNLNYVLCNLTITWEFFNILTFWLNSLLTVFYCIKVSSFTHHIFLWLRWRILRLFPWILLGSLMITCVTIIPSAIGNYIQIQLLTMEHLPRNSTVTDKLENFHQYQFQAHTVALVIPFILFLASTIFLMASLTKQIQHHSTGHCNPSMKARFTALRSLAVLFIVFTSYFLTILITIIGTLFDKRCWLWVWEAFVYAFILMHSTSLMLSSPTLKRILKGKC

SEQ ID NO: 23 - DNA, 1002 bp, Homo sapiens
  CDS (1)..(1002)
atgttgactctaactcgcatccgcactgtgtcctatgaagtcaggagtacatttctgttcatttcagtcctggagtttgcagtggggtttctgaccaatgccttcgttttcttggtgaatttttgggatgtagtgaagaggcagccactgagcaacagtgattgtgtgctgctgtgtctcagcatcagccggcttttcctgcatggactgctgttcctgagtgctatccagcttacccacttccagaagttgagtgaaccactgaaccacagctaccaagccatcatcatgctatggatgattgcaaaccaagccaacctctggcttgctgcctgcctcagcctgctttactgctccaagctcatccgtttctctcacaccttcctgatctgcttggcaagctgggtctccaggaagatctcccagatgctcctgggtattattctttgctcctgcatctgcactgtcctctgtgtttggtgcttttttagcagacctcacttcacagtcacaactgtgctattcatgaataacaatacaaggctcaactggcagattaaagatctcaatttattttattcctttctcttctgctatctgtggtctgtgcctcctttcctattgtttctggtttcttctgggatgctgactgtctccctgggaaggcacatgaggacaatgaaggtctataccagaaactctcgtgaccccagcctggaggcccacattaaagccctcaagtctcttgtctcctttttctgcttctttgtgatatcatcctgtgctgccttcatctctgtgcccctactgattctgtggcgcgacaaaataggggtgatggtttgtgttgggataatggcagcttgtccctctgggcatgcagccgtcctgatctcaggcaatgccaagttgaggagagctgtgatgaccattctgctctgggctcagagcagcctgaaggtaagagccgaccacaaggcagattcccggacactgtgctga

SEQ ID NO: 24 - protein, 333 aa, Homo sapiens [sequence omitted]

SEQ ID NO: 25 - DNA, 1017 bp, Homo sapiens
  CDS (1)..(1017)
atgctagggagatgttttcctccagacaccaaagagaagcaacagctcagaatgactaaactctgcgatcctgcagaaagtgaattgtcgccatttctcatcaccttaattttagcagttttacttgctgaatacctcattggtatcattgcaaatggtttcatcatggctatacatgcagctgaatgggttcaaaataaggcagtttccacaagtggcaggatcctggttttcctgagtgtatccagaatagctctccaaagcctcatgatgttagaaattaccatcagctcaacctccctaagtttttattctgaagacgctgtatattatgcattcaaaataagttttatattcttaaatttttgtagcctgtggtttgctgcctggctcagtttcttctactttgtgaagattgccaatttctcctacccccttttcctcaaactgaggtggagaattactggattgataccctggcttctgtggctgtccgtgtttatttccttcagtcacagcatgttctgcatcaacatctgcactgtgtattgtaacaattctttccctatccactcctccaactccactaagaaaacatacttgtctgagatcaatgtggtcggtctggcttttttctttaacctggggattgtgactcctctgatcatgttcatcctgacagccaccctgctgatcctctctctcaagagacacaccctacacatgggaagcaatgccacagggtccaacgaccccagcatggaggctcacatgggggccatcaaagctatcagctactttctcattctctacattttcaatgcagttgctctgtttatctacctgtccaacatgtttgacatcaacagtctgtggaataatttgtgccagatcatcatggctgcctaccctgccagccactcaattctactgattcaagataaccctgggctgagaagagcctggaagcggcttcagcttcgacttcatctttacccaaaagagtggactctgtga

SEQ ID NO: 26 - protein, 338 aa, Homo sapiens
MLGRCFPPDTKEKQQLRMTKLCDPAESELSPFLITLILAVLLAEYLIGIIANGFIMAIHAAEWVQNKAVSTSGRILVFLSVSRIALQSLMMLEITISSTSLSFYSEDAVYYAFKISFIFLNFCSLWFAAWLSFFYFVKIANFSYPLFLKLRWRITGLIPWLLWLSVFISFSHSMFCINICTVYCNNSFPIHSSNSTKKTYLSEINVVGLAFFFNLGIVTPLIMFILTATLLILSLKRHTLHMGSNATGSNDPSMEAHMGAIKAISYFLILYIFNAVALFIYLSNMFDINSLWNNLCQIIMAAYPASHSILLIQDNPGLRRAWKRLQLRLHLYPKEWTL

SEQ ID NO: 27 - DNA, 972 bp, Homo sapiens
  CDS (1)..(972)
atggcaacggtgaacacagatgccacagataaagacatatccaagttcaaggtcaccttcactttggtggtctccggaatagagtgcatcactggcatccttgggagtggcttcatcacggccatctatggggctgagtgggccaggggcaaaacactccccactggtgaccgcattatgttgatgctgagcttttccaggctcttgctacagatttggatgatgctggagaacattttcagtctgctattccgaattgtttataaccaaaactcagtgtatatcctcttcaaagtcatcactgtctttctgaaccattccaatctctggtttgctgcctggctcaaagtcttctattgtcttagaattgcaaacttcaatcatcctttgttcttcctgatgaagaggaaaatcatagtgctgatgccttggcttctcaggctgtcagtgttggtttccttaagcttcagctttcctctctcgagagatgtcttcaatgtgtatgtgaatagctccattcctatcccctcctccaactccacggagaagaagtacttctctgagaccaatatggtcaacctggtatttttctataacatggggatcttcgttcctctgatcatgttcatcctggcagccaccctgctgatcctctctctcaagagacacaccctacacatgggaagcaatgccacagggtccagggaccccagcatgaaggctcacataggggccatcaaagccaccagctactttctcatcctctacattttcaatgcaattgctctatttctttccacgtccaacatctttgacacttacagttcctggaatattttgtgcaagatcatcatggctgcctaccctgccggccactcagtacaactgatcttgggcaaccctgggctgagaagagcctggaagcggtttcagcaccaagttcctctttacctaaaagggcagactctgtga

SEQ ID NO: 28 - protein, 323 aa, Homo sapiens
MATVNTDATDKDISKFKVTFTLVVSGIECITGILGSGFITAIYGAEWARGKTLPTGDRIMLMLSFSRLLLQIWMMLENIFSLLFRIVYNQNSVYILFKVITVFLNHSNLWFAAWLKVFYCLRIANFNHPLFFLMKRKIIVLMPWLLRLSVLVSLSFSFPLSRDVFNVYVNSSIPIPSSNSTEKKYFSETNMVNLVFFYNMGIFVPLIMFILAATLLILSLKRHTLHMGSNATGSRDPSMKAHIGAIKATSYFLILYIFNAIALFLSTSNIFDTYSSWNILCKIIMAAYPAGHSVQLILGNPGLRRAWKRFQHQVPLYLKGQTL

SEQ ID NO: 29 - DNA, 924 bp, Homo sapiens
  CDS (1)..(924)
atgcaagcagcactgacggccttcttcgtgttgctctttagcctgctgagtcttctggggattgcagcgaatggcttcattgtgctggtgctgggcagggagtggctgcgatatggcaggttgctgcccttggatatgatcctcattagcttgggtgcctcccgcttctgcctgcagttggttgggacagtgcacaacttctactactctgcccagaaggtcgagtactctgggggtctcggccgacagttcttccatctacactggcacttcctgaactcagccaccttctggttttgcagctggctcagtgtcctgttctgtgtgaagattgctaacatcacacactccaccttcctgtggctgaagtggaggttcctagggtgggtgccctggctcctgttgggctctgtcctgatctccttcatcataaccctgctgtttttttgggtgaactaccctgtatatcaagaatttttaattagaaaattttctgggaacatgacctacaagtggaatacaaggatagaaacatactatttcccatccctgaaactggtcatctggtcaattcctttttctgtttttctggtctcaattatgctgttaattaattctctgaggaggcatactcagagaatgcagcacaacgggcacagcctgcaggaccccagcacccaggctcacaccagagctctgaagtccctcatctccttcctcattctttatgctctgtcctttctgtccctgatcattgatgccgcaaaatttatctccatgcagaacgacttttactggccatggcaaattgcagtctacctgtgcatatctgtccatcccttcatcctcatcttcagcaacctcaagcttcgaagcgtgttctcacagctcctgttgttggcaaggggcttctgggtggcctga

SEQ ID NO: 30 - protein, 307 aa, Homo sapiens
MQAALTAFFVLLFSLLSLLGIAANGFIVLVLGREWLRYGRLLPLDMILISLGASRFCLQLVGTVHNFYYSAQKVEYSGGLGRQFFHLHWHFLNSATFWFCSWLSVLFCVKIANITHSTFLWLKWRFLGWVPWLLLGSVLISFIITLLFFWVNYPVYQEFLIRKFSGNMTYKWNTRIETYYFPSLKLVIWSIPFSVFLVSIMLLINSLRRHTQRMQHNGHSLQDPSTQAHTRALKSLISFLILYALSFLSLIIDAAKFISMQNDFYWPWQIAVYLCISVHPFILIFSNLKLRSVFSQLLLLARGFWVA

SEQ ID NO: 31 - DNA, 945 bp, Homo sapiens
  CDS (1)..(945)
atggccaccgaattggacaaaatctttctgattctggcaatagcagaattcatcatcagcatgctggggaatgtgttcattggactggtaaactgctctgaagggatcaagaaccaaaaggtcttctcagctgacttcatcctcacctgcttggctatctccacaattggacaactgttggtgatactgtttgattcatttctagtgggacttgcttcacatttatataccacatatagactaggaaaaactgttattatgctttggcacatgactaatcacttgacaacctggcttgccacctgcctaagcattttctatttctttaagatagcccacttcccccactcccttttcctctggctgaggtggaggatgaacggaatgattgttatgcttcttatattgtctttgttcttactgatttttgacagtttagtgctagaaatatttattgatatctcactcaatataatagataaaagtaatctgactttatatttagatgaaagtaaaactctctatgataaactctctattttaaaaactcttctcagcttaaccagttttatccccttttctctgttcctgacctccttgctttttttatttctgtccttggtgagacatactagaaatttgaagctcagttccttgggctctagagactccagcacagaggcccataggagggccatgaaaatggtgatgtctttccttttcctcttcatagttcattttttttccttacaagtggccaatgggatattttttatgttgtggaacaacaagtacataaagtttgtcatgttagccttaaatgcctttccctcgtgccactcatttattctcattctgggaaacagcaagctgcgacagacagctgtgaggctactgtggcatcttaggaactatacaaaaacaccaaatgctttacctttgtag

SEQ ID NO: 32 - protein, 314 aa, Homo sapiens
MATELDKIFLILAIAEFIISMLGNVFIGLVNCSEGIKNQKVFSADFILTCLAISTIGQLLVILFDSFLVGLASHLYTTYRLGKTVIMLWHMTNHLTTWLATCLSIFYFFKIAHFPHSLFLWLRWRMNGMIVMLLILSLFLLIFDSLVLEIFIDISLNIIDKSNLTLYLDESKTLYDKLSILKTLLSLTSFIPFSLFLTSLLFLFLSLVRHTRNLKLSSLGSRDSSTEAHRRAMKMVMSFLFLFIVHFFSLQVANGIFFMLWNNKYIKFVMLALNAFPSCHSFILILGNSKLRQTAVRLLWHLRNYTKTPNALPL

SEQ ID NO: 33 - DNA, 930 bp, Homo sapiens
  CDS (1)..(930)
atgataacttttctacccatcattttttccagtctggtagtggttacatttgttattggaaattttgctaatggcttcatagcactggtaaattccattgagtcgttcaagagacaaaagatctcctttgctgaccaaattctcactgctctggcggtctccagagttggtttgctctgggtattattattaaactggtattcaactgtgttgaatccagcttttaatagtgtagaagtaagaactactgcttataatatctgggcagtgatcaaccatttcagcaactggcttgctactaccctcagcatattttatttgctcaagattgccaatttctccaactttatttttcttcacttaaagaggagagttaagagtgtcattctggtgatgttgttggggcctttgctatttttggcttgtcatctttttgtgataaacatgaatgagattgtgcggacaaaagaatttgaaggaaacatgacttggaagatcaaattgaagagtgcaatgtacttttcaaatatgactgtaaccatggtagcaaacttagtacccttcactctgaccctactatcttttatgctgttaatctgttctttgtgtaaacatctcaagaagatgcagctccgtggtaaaggatctcaagatcccagcacgaaggtccacataaaagctttgcaaactgtgatctccttcctcttgttatgtgccatttactttctgtccataatgatatcagtttggagttttggaagtctggaaaacaaacctgtcttcatgttctgcaaagctattagattcagctatccttcaatccacccattcatcctgatttggggaaacaagaagctaaagcagacttttctttcagttttttggcaaatgaggtactgggtgaaaggagagaagacttcatctccatag

SEQ ID NO: 34 - protein, 309 aa, Homo sapiens
MITFLPIIFSSLVVVTFVIGNFANGFIALVNSIESFKRQKISFADQILTALAVSRVGLLWVLLLNWYSTVLNPAFNSVEVRTTAYNIWAVINHFSNWLATTLSIFYLLKIANFSNFIFLHLKRRVKSVILVMLLGPLLFLACHLFVINMNEIVRTKEFEGNMTWKIKLKSAMYFSNMTVTMVANLVPFTLTLLSFMLLICSLCKHLKKMQLRGKGSQDPSTKVHIKALQTVISFLLLCAIYFLSIMISVWSFGSLENKPVFMFCKAIRFSYPSIHPFILIWGNKKLKQTFLSVFWQMRYWVKGEKTSSP

SEQ ID NO: 35 - DNA, 930 bp, Homo sapiens
  CDS (1)..(930)
atgacaacttttatacccatcattttttccagtgtggtagtggttctatttgttattggaaattttgctaatggcttcatagcattggtaaattccattgagcgggtcaagagacaaaagatctcttttgctgaccagattctcactgctctggcggtctccagagttggtttgctctgggtattattattaaattggtattcaactgtgtttaatccagctttttatagtgtagaagtaagaactactgcttataatgtctgggcagtaaccggccatttcagcaactggcttgctactagcctcagcatattttatttgctcaagattgccaatttctccaaccttatttttcttcacttaaagaggagagttaagagtgtcattctggtgatgctgttggggcctttactatttttggcttgtcaactttttgtgataaacatgaaagagattgtacggacaaaagaatatgaaggaaacatgacttggaagatcaaattgaggagtgcagtgtacctttcagatgcgactgtaaccacgctaggaaacttagtgcccttcactctgaccctgctatgttttttgctgttaatctgttctctgtgtaaacatctcaagaagatgcagctccatggtaaaggatctcaagatcccagcaccaaggtccacataaaagctttgcaaactgtgatctttttcctcttgttatgtgccgtttactttctgtccataatgatatcagtttggagttttgggagtctggaaaacaaacctgtcttcatgttctgcaaagctattagattcagctatccttcaatccacccattcatcctgatttggggaaacaagaagctaaagcagacttttctttcagttttgcggcaagtgaggtactgggtgaaaggagagaagccttcatctccatga

SEQ ID NO: 36 - protein, 309 aa, Homo sapiens
MTTFIPIIFSSVVVVLFVIGNFANGFIALVNSIERVKRQKISFADQILTALAVSRVGLLWVLLLNWYSTVFNPAFYSVEVRTTAYNVWAVTGHFSNWLATSLSIFYLLKIANFSNLIFLHLKRRVKSVILVMLLGPLLFLACQLFVINMKEIVRTKEYEGNMTWKIKLRSAVYLSDATVTTLGNLVPFTLTLLCFLLLICSLCKHLKKMQLHGKGSQDPSTKVHIKALQTVIFFLLLCAVYFLSIMISVWSFGSLENKPVFMFCKAIRFSYPSIHPFILIWGNKKLKQTFLSVLRQVRYWVKGEKPSSP

SEQ ID NO: 37 - DNA, 900 bp, Homo sapiens
  CDS (1)..(900)
atgataacttttctgcccatcatattttccattctagtagtggttacatttgttattggaaattttgctaatggcttcatagcgttggtaaattccaccgagtgggtgaagagacaaaagatctcctttgctgaccaaattgtcactgctctggcggtctccagagttggtttgctctgggtgttattattaaattggtattcaactgtgttgaatccagctttttgtagtgtagaattaagaactactgcttataatatctgggcagtaaccggccatttcagcaactggcctgctactagcctcagcatattttatttgctcaagattgccaatttctccaaccttatttttcttcgcttaaagaggagagttaagagtgtcattctggtggtgctgttggggcctttgctatttttggcttgtcatctttttgtggtaaacatgaatcagattgtatggacaaaagaatatgaaggaaacatgacttggaagatcaaattgaggcgtgcaatgtacctttcagatacgactgtaaccatgctagcaaacttagtaccctttactgtaaccctgatatcttttctgctgttagtctgttctctgtgtaaacatctcaagaagatgcagctccatggcaaaggatctcaagatcccagtaccaaggtccacataaaagttttgcaaactgtgatctccttcttcttgttacgtgccatttactttgtgtctgtaataatatcagtttggagttttaagaatctggaaaacaaacctgtcttcatgttctgccaagctattggattcagctgttcttcagcccacccgttcatcctgatttggggaaacaagaagctaaagcagacttatctttcagttttgtggcaaatgaggtactga

SEQ ID NO: 38 - protein, 299 aa, Homo sapiens
MITFLPIIFSILVVVTFVIGNFANGFIALVNSTEWVKRQKISFADQIVTALAVSRVGLLWVLLLNWYSTVLNPAFCSVELRTTAYNIWAVTGHFSNWPATSLSIFYLLKIANFSNLIFLRLKRRVKSVILVVLLGPLLFLACHLFVVNMNQIVWTKEYEGNMTWKIKLRRAMYLSDTTVTMLANLVPFTVTLISFLLLVCSLCKHLKKMQLHGKGSQDPSTKVHIKVLQTVISFFLLRAIYFVSVIISVWSFKNLENKPVFMFCQAIGFSCSSAHPFILIWGNKKLKQTYLSVLWQMRY

SEQ ID NO: 39 - DNA, 930 bp, Homo sapiens
  CDS (1)..(930)
atgataacttttctgcccatcattttttccattctaatagtggttacatttgtgattggaaattttgctaatggcttcatagcattggtaaattccattgagtggttcaagagacaaaagatctcttttgctgaccaaattctcactgctctggcagtctccagagttggtttactctgggtattagtattaaattggtatgcaactgagttgaatccagcttttaacagtatagaagtaagaattactgcttacaatgtctgggcagtaatcaaccatttcagcaactggcttgctactagcctcagcatattttatttgctcaagattgccaatttctccaaccttatttttcttcacttaaagaggagagttaagagtgttgttctggtgatactattggggcctttgctatttttggtttgtcatctttttgtgataaacatgaatcagattatatggacaaaagaatatgaaggaaacatgacttggaagatcaaactgaggagtgcaatgtacctttcaaatacaacggtaaccatcctagcaaacttagttcccttcactctgaccctgatatcttttctgctgttaatctgttctctgtgtaaacatctcaaaaagatgcagctccatggcaaaggatctcaagatcccagcatgaaggtccacataaaagctttgcaaactgtgacctccttcctcttgttatgtgccatttactttctgtccataatcatgtcagtttggagttttgagagtctggaaaacaaacctgtcttcatgttctgcgaagctattgcattcagctatccttcaacccacccattcatcctgatttggggaaacaagaagctaaagcagacttttctttcagttttgtggcatgtgaggtactgggtgaaaggagagaagccttcatcttcatag

SEQ ID NO: 40 - protein, 309 aa, Homo sapiens
MITFLPIIFSILIVVTFVIGNFANGFIALVNSIEWFKRQKISFADQILTALAVSRVGLLWVLVLNWYATELNPAFNSIEVRITAYNVWAVINHFSNWLATSLSIFYLLKIANFSNLIFLHLKRRVKSVVLVILLGPLLFLVCHLFVINMNQIIWTKEYEGNMTWKIKLRSAMYLSNTTVTILANLVPFTLTLISFLLLICSLCKHLKKMQLHGKGSQDPSMKVHIKALQTVTSFLLLCAIYFLSIIMSVWSFESLENKPVFMFCEAIAFSYPSTHPFILIWGNKKLKQTFLSVLWHVRYWVKGEKPSSS

SEQ ID NO: 41 - DNA, 960 bp, Homo sapiens
  CDS (1)..(960)
atgataacttttctgcccatcattttttccattctaatagtggttatatttgttattggaaattttgctaatggcttcatagcattggtaaattccattgagtgggtcaagagacaaaagatctcctttgttgaccaaattctcactgctctggcggtctccagagttggtttgctctgggtgttattactacattggtatgcaactcagttgaatccagctttttatagtgtagaagtaagaattactgcttataatgtctgggcagtaaccaaccatttcagcagctggcttgctactagcctcagcatgttttatttgctcaggattgccaatttctccaaccttatttttcttcgcataaagaggagagttaagagtgttgttctggtgatactgttggggcctttgctatttttggtttgtcatctttttgtgataaacatggatgagactgtatggacaaaagaatatgaaggaaacgtgacttggaagatcaaattgaggagtgcaatgtaccattcaaatatgactctaaccatgctagcaaactttgtacccctcactctgaccctgatatcttttctgctgttaatctgttctctgtgtaaacatctcaagaagatgcagctccatggcaaaggatctcaagatcccagcaccaaggtccacataaaagctttgcaaactgtgacctcctttcttctgttatgtgccatttactttctgtccatgatcatatcagtttgtaattttgggaggctggaaaagcaacctgtcttcatgttctgccaagctattatattcagctatccttcaacccacccattcatcctgattttgggaaacaagaagctaaagcagatttttctttcagttttgcggcatgtgaggtactgggtgaaagacagaagccttcgtctccatagattcacaagaggggcattgtgtgtcttctag

SEQ ID NO: 42 - protein, 319 aa, Homo sapiens
MITFLPIIFSILIVVIFVIGNFANGFIALVNSIEWVKRQKISFVDQILTALAVSRVGLLWVLLLHWYATQLNPAFYSVEVRITAYNVWAVTNHFSSWLATSLSMFYLLRIANFSNLIFLRIKRRVKSVVLVILLGPLLFLVCHLFVINMDETVWTKEYEGNVTWKIKLRSAMYHSNMTLTMLANFVPLTLTLISFLLLICSLCKHLKKMQLHGKGSQDPSTKVHIKALQTVTSFLLLCAIYFLSMIISVCNFGRLEKQPVFMFCQAIIFSYPSTHPFILILGNKKLKQIFLSVLRHVRYWVKDRSLRLHRFTRGALCVF

SEQ ID NO: 43 - DNA, 900 bp, Homo sapiens
  CDS (1)..(900)
atgatgtgttttctgctcatcatttcatcaattctggtagtgtttgcatttgttcttggaaatgttgccaatggcttcatagccctagtaaatgtcattgactgggttaacacacgaaagatctcctcagctgagcaaattctcactgctctggtggtctccagaattggtttactctgggtcatgttattcctttggtatgcaactgtgtttaattctgctttatatggtttagaagtaagaattgttgcttctaatgcctgggctgtaacgaaccatttcagcatgtggcttgctgctagcctcagcatattttgtttgctcaagattgccaatttctccaaccttatttctctccacctaaagaagagaattaagagtgttgttctggtgatactgttggggcccttggtatttctgatttgtaatcttgctgtgataaccatggatgagagagtgtggacaaaagaatatgaaggaaatgtgacttggaagatcaaattgaggaatgcaatacacctttcaagcttgactgtaactactctagcaaacctcataccctttactctgagcctaatatgttttctgctgttaatctgttctctttgtaaacatctcaagaagatgcggctccatagcaaaggatctcaagatcccagcaccaaggtccatataaaagctttgcaaactgtgacctccttcctcatgttatttgccatttactttctgtgtataatcacatcaacttggaatcttaggacacagcagagcaaacttgtactcctgctttgccaaactgttgcaatcatgtatccttcattccactcattcatcctgattatgggaagtaggaagctaaaacagacctttctttcagttttgtggcagatgacacgctga

SEQ ID NO: 44 - protein, 299 aa, Homo sapiens
MMCFLLIISSILVVFAFVLGNVANGFIALVNVIDWVNTRKISSAEQILTALVVSRIGLLWVMLFLWYATVFNSALYGLEVRIVASNAWAVTNHFSMWLAASLSIFCLLKIANFSNLISLHLKKRIKSVVLVILLGPLVFLICNLAVITMDERVWTKEYEGNVTWKIKLRNAIHLSSLTVTTLANLIPFTLSLICFLLLICSLCKHLKKMRLHSKGSQDPSTKVHIKALQTVTSFLMLFAIYFLCIITSTWNLRTQQSKLVLLLCQTVAIMYPSFHSFILIMGSRKLKQTFLSVLWQMTR

SEQ ID NO: 45 - DNA, 930 bp, Homo sapiens
  CDS (1)..(930)
atgatgagttttctacacattgttttttccattctagtagtggttgcatttattcttggaaattttgccaatggctttatagcactgataaatttcattgcctgggtcaagagacaaaagatctcctcagctgatcaaattattgctgctctggcagtctccagagttggtttgctctgggtaatattattacattggtattcaactgtgttgaatccaacttcatctaatttaaaagtaataatttttatttctaatgcctgggcagtaaccaatcatttcagcatctggcttgctactagcctcagcatattttatttgctcaagatcgtcaatttctccagacttatttttcatcacttaaaaaggaaggctaagagtgtagttctggtgatagtgttggggtctttgttctttttggtttgtcaccttgtgatgaaacacacgtatataaatgtgtggacagaagaatgtgaaggaaacgtaacttggaagatcaaactgaggaatgcaatgcacctttccaacttgactgtagccatgctagcaaacttgataccattcactctgaccctgatatcttttctgctgttaatctactctctgtgtaaacatctgaagaagatgcagctccatggcaaaggatctcaagatcccagcaccaagatccacataaaagctctgcaaactgtgacctccttcctcatattacttgccatttactttctgtgtctaatcatatcgttttggaattttaagatgcgaccaaaagaaattgtcttaatgctttgccaagcttttggaatcatatatccatcattccactcattcattctgatttgggggaacaagacgctaaagcagacctttctttcagttttgtggcaggtgacttgctgggcaaaaggacagaaccagtcaactccatag

SEQ ID NO: 46 - protein, 309 aa, Homo sapiens
MMSFLHIVFSILVVVAFILGNFANGFIALINFIAWVKRQKISSADQIIAALAVSRVGLLWVILLHWYSTVLNPTSSNLKVIIFISNAWAVTNHFSIWLATSLSIFYLLKIVNFSRLIFHHLKRKAKSVVLVIVLGSLFFLVCHLVMKHTYINVWTEECEGNVTWKIKLRNAMHLSNLTVAMLANLIPFTLTLISFLLLIYSLCKHLKKMQLHGKGSQDPSTKIHIKALQTVTSFLILLAIYFLCLIISFWNFKMRPKEIVLMLCQAFGIIYPSFHSFILIWGNKTLKQTFLSVLWQVTCWAKGQNQSTP

SEQ ID NO: 47 - DNA, 900 bp, Homo sapiens
  CDS (1)..(900)
atgataacttttctatacatttttttttcaattctaataatggttttatttgttctcggaaactttgccaatggcttcatagcactggtaaatttcattgactgggtgaagagaaaaaagatctcctcagctgaccaaattctcactgctctggcggtctccagaattggtttgctctgggcattattattaaattggtatttaactgtgttgaatccagctttttatagtgtagaattaagaattacttcttataatgcctgggttgtaaccaaccatttcagcatgtggcttgctgctaacctcagcatattttatttgctcaagattgccaatttctccaaccttctttttcttcatttaaagaggagagttaggagtgtcattctggtgatactgttggggactttgatatttttggtttgtcatcttcttgtggcaaacatggatgagagtatgtgggcagaagaatatgaaggaaacatgactgggaagatgaaattgaggaatacagtacatctttcatatttgactgtaactaccctatggagcttcataccctttactctgtccctgatatcttttctgatgctaatctgttctctgtgtaaacatctcaagaagatgcagctccatggagaaggatcgcaagatctcagcaccaaggtccacataaaagctttgcaaactctgatctccttcctcttgttatgtgccattttctttctattcctaatcgtttcggtttggagtcctaggaggctgcggaatgacccggttgtcatggttagcaaggctgttggaaacatatatcttgcattcgactcattcatcctaatttggagaaccaagaagctaaaacacacctttcttttgattttgtgtcagattaggtgctga

SEQ ID NO: 48 - protein, 299 aa, Homo sapiens
MITFLYIFFSILIMVLFVLGNFANGFIALVNFIDWVKRKKISSADQILTALAVSRIGLLWALLLNWYLTVLNPAFYSVELRITSYNAWVVTNHFSMWLAANLSIFYLLKIANFSNLLFLHLKRRVRSVILVILLGTLIFLVCHLLVANMDESMWAEEYEGNMTGKMKLRNTVHLSYLTVTTLWSFIPFTLSLISFLMLICSLCKHLKKMQLHGEGSQDLSTKVHIKALQTLISFLLLCAIFFLFLIVSVWSPRRLRNDPVVMVSKAVGNIYLAFDSFILIWRTKKLKHTFLLILCQIRC

SEQ ID NO: 49 - DNA, 957 bp, Homo sapiens
  CDS (1)..(957)
atgaatggagaccacatggttctaggatcttcggtgactgacaagaaggccatcatcttggttaccattttactccttttacgcctggtagcaatagcaggcaatggcttcatcactgctgctctgggcgtggagtgggtgctacggagaatgttgttgccttgtgataagttattggttagcctaggggcctctcgcttctgtctgcagtcagtggtaatgggtaagaccatttatgttttcttgcatccgatggccttcccatacaaccctgtactgcagtttctagctttccagtgggacttcctgaatgctgccaccttatggtcctctacctggctcagtgtcttctattgtgtgaaaattgctaccttcacccaccctgtcttcttctggctaaagcacaagttgtctgggtggctaccatggatgctcttcagctctgtagggctctccagcttcaccaccattctatttttcataggcaaccacagaatgtatcagaactatttaaggaaccatctacaaccttggaatgtcactggcgatagcatacggagctactgtgagaaattctatctcttccctctaaaaatgattacttggacaatgcccactgctgtctttttcatttgcatgattttgctcatcacatctctgggaagacacaggaagaaggctctccttacaacctcaggattccgagagcccagtgtgcaggcacacataaaggctctgctggctctcctctcttttgccatgctcttcatctcatatttcctgtcactggtgttcagtgctgcaggtatttttccacctctggactttaaattctgggtgtgggagtcagtgatttatctgtgtgcagcagttcaccccatcattctgctcttcagcaactgcaggctgagagctgtgctgaagagtcgtcgttcctcaaggtgtgggacaccttga

SEQ ID NO: 50 - protein, 318 aa, Homo sapiens
MNGDHMVLGSSVTDKKAIILVTILLLLRLVAIAGNGFITAALGVEWVLRRMLLPCDKLLVSLGASRFCLQSVVMGKTIYVFLHPMAFPYNPVLQFLAFQWDFLNAATLWSSTWLSVFYCVKIATFTHPVFFWLKHKLSGWLPWMLFSSVGLSSFTTILFFIGNHRMYQNYLRNHLQPWNVTGDSIRSYCEKFYLFPLKMITWTMPTAVFFICMILLITSLGRHRKKALLTTSGFREPSVQAHIKALLALLSFAMLFISYFLSLVFSAAGIFPPLDFKFWVWESVIYLCAAVHPIILLFSNCRLRAVLKSRRSSRCGTP

SEQ ID NO: 51 - DNA, 948 bp, Homo sapiens
  CDS (1)..(948)
atgcagccagaatctggggccaatggaacagtcattgctgagttcatcctgctgggcttgctggaggcgccagggctgcagccagttgtctttgtgctcttcctctttgcctacctggtcacggtcaggggcaacctcagcatcctggcagctgtcttggtggagcccaaactccacacccccatgtacttcttcctggggaacctatcagtgctggatgttgggtgcatcagcgtcactgttccatcaatgttgagtcgtctcctgtcccgcaagcgtgcagttccctgtggggcctgccttacccagctcttcttcttccatctgttcgttggagtggactgcttcctgctgaccgccatggcctatgaccgattcctggccatctgccggcccctcacctacagcacccgcatgagtcagacagtccagaggatgttggtggctgcgtcctgggcttgtgctttcaccaacgcactgacccacactgtggccatgtccacgctcaacttctgtggccccaatgtgatcaatcacttctactgtgacctcccacagctcttccagctctcctgctccagcacccaactcaatgagctgctgctttttgctgtgggttttataatggcaggtacccccatggctctcattgtcatctcctatatccacgtggcagctgcagtcctgcgaattcgctctgtagagggcaggaagaaagccttctccacatgtggctcccacctcactgtggttgccatattctatggttcaggtatctttaactatatgcgactgggttcaaccaagctttcagacaaggataaagctgttggaattttcaacactgtcatcaatcccatgctgaacccaatcatctacagcttcagaaaccctgatgtgcagagtgccatctggaggatgctcacagggaggcggtcactggcttga

SEQ ID NO: 52 - protein, 315 aa, Homo sapiens
MQPESGANGTVIAEFILLGLLEAPGLQPVVFVLFLFAYLVTVRGNLSILAAVLVEPKLHTPMYFFLGNLSVLDVGCISVTVPSMLSRLLSRKRAVPCGACLTQLFFFHLFVGVDCFLLTAMAYDRFLAICRPLTYSTRMSQTVQRMLVAASWACAFTNALTHTVAMSTLNFCGPNVINHFYCDLPQLFQLSCSSTQLNELLLFAVGFIMAGTPMALIVISYIHVAAAVLRIRSVEGRKKAFSTCGSHLTVVAIFYGSGIFNYMRLGSTKLSDKDKAVGIFNTVINPMLNPIIYSFRNPDVQSAIWRMLTGRRSLA

SEQ ID NO: 53 - DNA, 939 bp, Homo sapiens
  CDS (1)..(939)
atggatggaggcaaccagagtgaaggttcagagttccttctcctggggatgtcagagagtcctgagcagcagcggatcctgttttggatgttcctgtccatgtacctggtcacggtggtgggaaatgtgctcatcatcctggccatcagctctgattcccgcctgcacacccccgtgtacttcttcctggccaacctctccttcactgacctcttctttgtcaccaacacaatccccaagatgctggtgaacctccagtcccataacaaagccatctcctatgcagggtgtctgacacagctctacttcctggtctccttggtggccctggacaacctcatcctggctgtgatggcatatgaccgctatgtggccatctgctgccccctccactacaccacagccatgagccctaagctctgtatcttactcctttccttgtgttgggtcctatccgtcctctatggcctcatacacaccctcctcatgaccagagtgaccttctgtgggtcacgaaaaatccactacatcttctgtgagatgtatgtattgctgaggatggcatgttccaacattcagattaatcacacagtgctgattgccacaggctgcttcatcttcctcattccctttggattcgtgatcatttcctatgtgctgattatcagagccatcctcagaataccctcagtctctaagaaatacaaagccttctccacctgtgcctcccatttgggtgcagtctccctcttctatgggacactttgtatggtatacctaaagcccctccatacctactctgtgaaggactcagtagccacagtgatgtatgctgtggtgacacccatgatgaatcccttcatctacagcctgaggaacaaggacatgcatggggctctgggaagactcctagataaacactttaagaggctgacatga

SEQ ID NO: 54 - protein, 312 aa, Homo sapiens
MDGGNQSEGSEFLLLGMSESPEQQRILFWMFLSMYLVTVVGNVLIILAISSDSRLHTPVYFFLANLSFTDLFFVTNTIPKMLVNLQSHNKAISYAGCLTQLYFLVSLVALDNLILAVMAYDRYVAICCPLHYTTAMSPKLCILLLSLCWVLSVLYGLIHTLLMTRVTFCGSRKIHYIFCEMYVLLRMACSNIQINHTVLIATGCFIFLIPFGFVIISYVLIIRAILRIPSVSKKYKAFSTCASHLGAVSLFYGTLCMVYLKPLHTYSVKDSVATVMYAVVTPMMNPFIYSLRNKDMHGALGRLLDKHFKRLT

SEQ ID NO: 55 - DNA, 939 bp, Homo sapiens
  CDS (1)..(939)
atgatgattaaaaaaaatgcaagttcggaagacttctttattctacttggattttctaattggcctcagctggaagtagttctctttgtggttatcttgatcttctacctgatgacactgacaggaaacctgttcatcatcatcctgtcatacgtggactcccatctccacacaccaatgtacttcttcctttcaaacctctcatttctggatctctgctacaccaccagctctatccctcagttgctggtgaatctccggggcccggaaaagaccatctcgtatgctggttgcatggttcaactttactttgttcttgcactgggaatcacagagtgtgtcctactggtggtgatgtcatatgatcgttatgtagctgtgtgtagacctttgcattacactgtcctcatgcaccctcgtttctgccacttgttggttgcggcttcttgggtaattggttttactatctcagcacttcattcctcctttactttctgggtacccctttgtggacatcgcctagtggatcacttcttctgtgaagttccagcacttctgcgtttatcatgtgttgacacccatgcaaatgagctgaccctcatggtcatgagctccatttttgttctcatacctctcattctcattctcactacctatggtgccattgcccgggctgtactgagcatgcaatcaaccactgggcttcagaaagtgtttaggacatgtggagcccatcttatggttgtatctctctttttcattccagtcatgtgcatgtatctccagccaccatcagaaaattctcctgatcagggcaagttcattgccctcttttatactgttgtcacaccgagtcttaatcctctaatctacactctcagaaacaagcatgtaaaaggggcagcgaagagactattggggtgggagtgggggaagtga

SEQ ID NO: 56 - protein, 312 aa, Homo sapiens
MMIKKNASSEDFFILLGFSNWPQLEVVLFVVILIFYLMTLTGNLFIIILSYVDSHLHTPMYFFLSNLSFLDLCYTTSSIPQLLVNLRGPEKTISYAGCMVQLYFVLALGITECVLLVVMSYDRYVAVCRPLHYTVLMHPRFCHLLVAASWVIGFTISALHSSFTFWVPLCGHRLVDHFFCEVPALLRLSCVDTHANELTLMVMSSIFVLIPLILILTTYGAIARAVLSMQSTTGLQKVFRTCGAHLMVVSLFFIPVMCMYLQPPSENSPDQGKFIALFYTVVTPSLNPLIYTLRNKHVKGAAKRLLGWEWGK

SEQ ID NO: 57 - DNA, 963 bp, Homo sapiens
  CDS (1)..(963)
atggaccaaagcaattatagttctttacatggttttattctgcttggcttctctaaccatccaaaaatggagatgatcctgtcaggagttgtcgccatcttctacttaattacattggtgggtaacacagccatcattcttgcatctctcctggattcccagcttcatacaccaatgtactttttcctcagaaatttatctttcctagatctatgtttcacaaccagcatcatccctcagatgctggtcaacttgtggggacctgataagaccatcagctatgtgggttgtatcatccaactctatgtttacatgtggttgggctcagttgagtgccttctcctggctgttatgtcctatgatcgttttacagctatatgtaagcccttgcattattttgtagtcatgaacccacatctatgtctaaagatgattatcatgatctggagtattagtttggccaattctgtagtattatgtacactcactctgaatttgcccacatgtggaaacaacattctggatcatttcttgtgtgagttgccagctctggtcaagatagcttgtgtagacaccacaacagttgaaatgtctgttttcgctttaggcattataattgtcctcacacctctcatccttattcttatatcctatggctacattgccaaagctgtgctgagaacgaagtcaaaagcaagccagcgaaaagcaatgaatacctgtggatctcatcttactgtagtgtctatgttctatggaactattatctacatgtacctgcaaccaggtaacagggcttccaaagaccagggcaagttcctcaccctcttttacaccgtcatcactccaagtctcaacccgctcatttacaccttaagaaataaggacatgaaggatgccctgaagaaactgatgagatttcaccacaaatctacaaaaataaagaggaattgcaagtcatag

SEQ ID NO: 58 - protein, 320 aa, Homo sapiens
MDQSNYSSLHGFILLGFSNHPKMEMILSGVVAIFYLITLVGNTAIILASLLDSQLHTPMYFFLRNLSFLDLCFTTSIIPQMLVNLWGPDKTISYVGCIIQLYVYMWLGSVECLLLAVMSYDRFTAICKPLHYFVVMNPHLCLKMIIMIWSISLANSVVLCTLTLNLPTCGNNILDHFLCELPALVKIACVDTTTVEMSVFALGIIIVLTPLILILISYGYIAKAVLRTKSKASQRKAMNTCGSHLTVVSMFYGTIIYMYLQPGNRASKDQGKFLTLFYTVITPSLNPLIYTLRNKDMKDALKKLMRFHHKSTKIKRNCKS

SEQ ID NO: 59 - DNA, 2526 bp, Homo sapiens
  CDS (1)..(2526)
atgctgctctgcacggctcgcctggtcggcctgcagcttctcatttcctgctgctgggcctttgcctgccatagcacggagtcttctcctgacttcaccctccccggagattacctcctggcaggcctgttccctctccattctggctgtctgcaggtgaggcacagacccgaggtgaccctgtgtgacaggtcttgtagcttcaatgagcatggctaccacctcttccaggctatgcggcttggggttgaggagataaacaactccacggccctgctgcccaacatcaccctggggtaccagctgtatgatgtgtgttctgactctgccaatgtgtatgccacgctgagagtgctctccctgccagggcaacaccacatagagctccaaggagaccttctccactattcccctacggtgctggcagtgattgggcctgacagcaccaaccgtgctgccaccacagccgccctgctgagccctttcctggtgcccatgattagctatgcggccagcagcgagacgctcagcgtgaagcggcagtatccctctttcctgcgcaccatccccaatgacaagtaccaggtggagaccatggtgctgctgctgcagaagttcgggtggacctggatctctctggttggcagcagtgacgactatgggcagctaggggtgcaggcactggagaaccaggccactggtcaggggatctgcattgctttcaaggacatcatgcccttctctgcccaggtgggcgatgagaggatgcagtgcctcatgcgccacctggcccaggccggggccaccgtcgtggttgttttttccagccggcagttggccagggtgtttttcgagtccgtggtgctgaccaacctgactggcaaggtgtgggtcgcctcagaagcctgggccctctccaggcacatcactggggtgcccgggatccagcgcattgggatggtgctgggcgtggccatccagaagagggctgtccctggcctgaaggcgtttgaagaagcctatgcccgggcagacaagaaggcccctaggccttgccacaagggctcctggtgcagcagcaatcagctctgcagagaatgccaagctttcatggcacacacgatgcccaagctcaaagccttctccatgagttctgcctacaacgcataccgggctgtgtatgcggtggcccatggcctccaccagctcctgggctgtgcctctggagcttgttccaggggccgagtctacccctggcagcttttggagcagatccacaaggtgcatttccttctacacaaggacactgtggcgtttaatgacaacagagatcccctcagtagctataacataattgcctgggactggaatggacccaagtggaccttcacggtcctcggttcctccacatggtctccagttcagctaaacataaatgagaccaaaatccagtggcacggaaaggacaaccaggtgcctaagtctgtgtgttccagcgactgtcttgaagggcaccagcgagtggttacgggtttccatcactgctgctttgagtgtgtgccctgtggggctgggaccttcctcaacaagagtgacctctacagatgccagccttgtgggaaagaagagtgggcacctgagggaagccagacctgcttcccgcgcactgtggtgtttttggctttgcgtgagcacacctcttgggtgctgctggcagctaacacgctgctgctgctgctgctgcttgggactgctggcctgtttgcctggcacctagacacccctgtggtgaggtcagcagggggccgcctgtgctttcttatgctgggctccctggcagcaggtagtggcagcctctatggcttctttggggaacccacaaggcctgcgtgcttgctacgccaggccctctttgcccttggtttcaccatcttcctgtcctgcctgacagttcgctcattccaactaatcatcatcttcaagttttccaccaaggtacctacattctaccacgcctgggtccaaaaccacggtgctggcctgtttgtgatgatcagctcagcggcccagctgcttatctgtctaacttggctggtggtgtggaccccactgcctgctagggaataccagcgcttcccccatctggtgatgcttgagtgcacagagaccaactccctgggcttcatactggccttcctctacaatggcctcctctccatcagtgcctttgcctgcagctacctgggtaaggacttgccagagaactacaacgaggccaaatgtgtcaccttcagcctgctcttcaacttcgtgtcctggatcgccttcttcaccacggccagcgtctacgacggcaagtacctgcctgcggccaacatgatggctgggctgagcagcctgagcagcggcttcggtgggtattttctgcctaagtgctacgtgatcctctgccgcccagacctcaacagcacagagcacttccaggcctccattcaggactacacgaggcgctgcggctccacctga

SEQ ID NO: 60 - protein, 841 aa, Homo sapiens
MLLCTARLVGLQLLISCCWAFACHSTESSPDFTLPGDYLLAGLFPLHSGCLQVRHRPEVTLCDRSCSFNEHGYHLFQAMRLGVEEINNSTALLPNITLGYQLYDVCSDSANVYATLRVLSLPGQHHIELQGDLLHYSPTVLAVIGPDSTNRAATTAALLSPFLVPMISYAASSETLSVKRQYPSFLRTIPNDKYQVETMVLLLQKFGWTWISLVGSSDDYGQLGVQALENQATGQGICIAFKDIMPFSAQVGDERMQCLMRHLAQAGATVVVVFSSRQLARVFFESVVLTNLTGKVWVASEAWALSRHITGVPGIQRIGMVLGVAIQKRAVPGLKAFEEAYARADKKAPRPCHKGSWCSSNQLCRECQAFMAHTMPKLKAFSMSSAYNAYRAVYAVAHGLHQLLGCASGACSRGRVYPWQLLEQIHKVHFLLHKDTVAFNDNRDPLSSYNIIAWDWNGPKWTFTVLGSSTWSPVQLNINETKIQWHGKDNQVPKSVCSSDCLEGHQRVVTGFHHCCFECVPCGAGTFLNKSDLYRCQPCGKEEWAPEGSQTCFPRTVVFLALREHTSWVLLAANTLLLLLLLGTAGLFAWHLDTPVVRSAGGRLCFLMLGSLAAGSGSLYGFFGEPTRPACLLRQALFALGFTIFLSCLTVRSFQLIIIFKFSTKVPTFYHAWVQNHGAGLFVMISSAAQLLICLTWLVVWTPLPAREYQRFPHLVMLECTETNSLGFILAFLYNGLLSISAFACSYLGKDLPENYNEAKCVTFSLLFNFVSWIAFFTTASVYDGKYLPAANMMAGLSSLSSGFGGYFLPKCYVILCRPDLNSTEHFQASIQDYTRRCGST

SEQ ID NO: 61 - DNA, 2520 bp, Homo sapiens
  CDS (1)..(2520)
atggggcccagggcaaagaccatctcctccctgttcttcctcctatgggtcctggctgagccggctgagaactcggacttctacctgcctggggattacctcctgggtggcctcttctccctccatgccaacatgaagggcattgttcaccttaacttcctgcaggtgcccatgtgcaaggagtatgaagtgaaggtgataggctacaacctcatgcaggccatgcgctttgcggtggaggagatcaacaatgacagcagcctgctgcctggtgtgctgctgggctatgagatcgtggatgtgtgctacatctccaacaatgtccagccggtgctctacttcctggcacacgaggacaacctccttcccatccaagaggactacagtaactacatttcccgtgtggtggctgtcattggccctgacaactccgagtctgtcatgactgtggccaacttcctctccctatttctccttccacagatcacctacagcgccatcagcgatgagctgcgagacaaggtgcgcttcccggctttgctgcgtaccacacccagcgccgaccaccacatcgaggccatggtgcagctgatgctgcacttccgctggaactggatcattgtgctggtgagcagcgacacctatggccgcgacaatggccagctgcttggcgagcgcgtggcccggcgcgacatctgcatcgccttccaggagacgctgcccacactgcagcccaaccagaacatgacgtcagaggagcgccagcgcctggtgaccattgtggacaagctgcagcagagcacagcgcgcgtcgtggtcgtgttctcgcccgacctgaccctgtaccacttcttcaatgaggtgctgcgccagaacttcactggcgccgtgtggatcgcctccgagtcctgggccatcgacccggtcctgcacaacctcacggagctgcgccacttgggcaccttcctgggcatcaccatccagagcgtgcccatcccgggcttcagtgagttccgcgagtggggcccacaggctgggccgccacccctcagcaggaccagccagagctatacctgcaaccaggagtgcgacaactgcctgaacgccaccttgtccttcaacaccattctcaggctctctggggagcgtgtcgtctacagcgtgtactctgcggtctatgctgtggcccatgccctgcacagcctcctcggctgtgacaaaagcacctgcaccaagagggtggtctacccctggcagctgcttgaggagatctggaaggtcaacttcactctcctggaccaccaaatcttcttcgacccgcaaggggacgtggctctgcacttggagattgtccagtggcaatgggaccggagccagaatcccttccagagcgtcgcctcctactaccccctgcagcgacagctgaagaacatccaagacatctcctggcacaccatcaacaacacgatccctatgtccatgtgttccaagaggtgccagtcagggcaaaagaagaagcctgtgggcatccacgtctgctgcttcgagtgcatcgactgccttcccggcaccttcctcaaccacactgaagatgaatatgaatgccaggcctgcccgaataacgagtggtcctaccagagtgagacctcctgcttcaagcggcagctggtcttcctggaatggcatgaggcacccaccatcgctgtggccctgctggccgccctgggcttcctcagcaccctggccatcctggtgatattctggaggcacttccagacacccatagttcgctcggctgggggccccatgtgcttcctgatgctgacactgctgctggtggcatacatggtggtcccggtgtacgtggggccgcccaaggtctccacctgcctctgccgccaggccctctttcccctctgcttcacaatctgcatctcctgtatcgccgtgcgttctttccagatcgtctgcgccttcaagatggccagccgcttcccacgcgcctacagctactgggtccgctaccaggggccctacgtctctatggcatttatcacggtactcaaaatggtcattgtggtaattggcatgctggccacgggcctcagtcccaccacccgtactgaccccgatgaccccaagatcacaattgtctcctgtaaccccaactaccgcaacagcctgctgttcaacaccagcctggacctgctgctctcagtggtgggtttcagcttcgcctacatgggcaaagagctgcccaccaactacaacgaggccaagttcatcaccctcagcatgaccttctatttcacctcatccgtctccctctgcaccttcatgtctgcctacagcggggtgctggtcaccatcgtggacctcttggtcactgtgctcaacctcctggccatcagcctgggctacttcggccccaagtgctacatgatcctcttctacccggagcgcaacacgcccgcctacttcaacagcatgatccagggctacaccatgaggagggactag

SEQ ID NO: 62 - protein, 839 aa, Homo sapiens
MGPRAKTISSLFFLLWVLAEPAENSDFYLPGDYLLGGLFSLHANMKGIVHLNFLQVPMCKEYEVKVIGYNLMQAMRFAVEEINNDSSLLPGVLLGYEIVDVCYISNNVQPVLYFLAHEDNLLPIQEDYSNYISRVVAVIGPDNSESVMTVANFLSLFLLPQITYSAISDELRDKVRFPALLRTTPSADHHIEAMVQLMLHFRWNWIIVLVSSDTYGRDNGQLLGERVARRDICIAFQETLPTLQPNQNMTSEERQRLVTIVDKLQQSTARVVVVFSPDLTLYHFFNEVLRQNFTGAVWIASESWAIDPVLHNLTELRHLGTFLGITIQSVPIPGFSEFREWGPQAGPPPLSRTSQSYTCNQECDNCLNATLSFNTILRLSGERVVYSVYSAVYAVAHALHSLLGCDKSTCTKRVVYPWQLLEEIWKVNFTLLDHQIFFDPQGDVALHLEIVQWQWDRSQNPFQSVASYYPLQRQLKNIQDISWHTINNTIPMSMCSKRCQSGQKKKPVGIHVCCFECIDCLPGTFLNHTEDEYECQACPNNEWSYQSETSCFKRQLVFLEWHEAPTIAVALLAALGFLSTLAILVIFWRHFQTPIVRSAGGPMCFLMLTLLLVAYMVVPVYVGPPKVSTCLCRQALFPLCFTICISCIAVRSFQIVCAFKMASRFPRAYSYWVRYQGPYVSMAFITVLKMVIVVIGMLATGLSPTTRTDPDDPKITIVSCNPNYRNSLLFNTSLDLLLSVVGFSFAYMGKELPTNYNEAKFITLSMTFYFTSSVSLCTFMSAYSGVLVTIVDLLVTVLNLLAISLGYFGPKCYMILFYPERNTPAYFNSMIQGYTMRRD

SEQ ID NO: 63 - DNA, 2559 bp, Homo sapiens
  CDS (1)..(2559)
atgctgggccctgctgtcctgggcctcagcctctgggctctcctgcaccctgggacgggggccccattgtgcctgtcacagcaacttaggatgaagggggactacgtgctgggggggctgttccccctgggcgaggccgaggaggctggcctccgcagccggacacggcccagcagccctgtgtgcaccaggttctcctcaaacggcctgctctgggcactggccatgaaaatggccgtggaggagatcaacaacaagtcggatctgctgcccgggctgcgcctgggctacgacctctttgatacgtgctcggagcctgtggtggccatgaagcccagcctcatgttcctggccaaggcaggcagccgcgacatcgccgcctactgcaactacacgcagtaccagccccgtgtgctggctgtcatcgggccccactcgtcagagctcgccatggtcaccggcaagttcttcagcttcttcctcatgccccaggtcagctacggtgctagcatggagctgctgagcgcccgggagaccttcccctccttcttccgcaccgtgcccagcgaccgtgtgcagctgacggccgccgcggagctgctgcaggagttcggctggaactgggtggccgccctgggcagcgacgacgagtacggccggcagggcctgagcatcttctcggccctggccgcggcacgcggcatctgcatcgcgcacgagggcctggtgccgctgccccgtgccgatgactcgcggctggggaaggtgcaggacgtcctgcaccaggtgaaccagagcagcgtgcaggtggtgctgctgttcgcctccgtgcacgccgcccacgccctcttcaactacagcatcagcagcaggctctcgcccaaggtgtgggtggccagcgaggcctggctgacctctgacctggtcatggggctgcccggcatggcccagatgggcacggtgcttggcttcctccagaggggtgcccagctgcacgagttcccccagtacgtgaagacgcacctggccctggccaccgacccggccttctgctctgccctgggcgagagggagcagggtctggaggaggacgtggtgggccagcgctgcccgcagtgtgactgcatcacgctgcagaacgtgagcgcagggctaaatcaccaccagacgttctctgtctacgcagctgtgtatagcgtggcccaggccctgcacaacactcttcagtgcaacgcctcaggctgccccgcgcaggaccccgtgaagccctggcagctcctggagaacatgtacaacctgaccttccacgtgggcgggctgccgctgcggttcgacagcagcggaaacgtggacatggagtacgacctgaagctgtgggtgtggcagggctcagtgcccaggctccacgacgtgggcaggttcaacggcagcctcaggacagagcgcctgaagatccgctggcacacgtctgacaaccagaagcccgtgtcccggtgctcgcggcagtgccaggagggccaggtgcgccgggtcaaggggttccactcctgctgctacgactgtgtggactgcgaggcgggcagctaccggcaaaacccagacgacatcgcctgcaccttttgtggccaggatgagtggtccccggagcgaagcacacgctgcttccgccgcaggtctcggttcctggcatggggcgagccggctgtgctgctgctgctcctgctgctgagcctggcgctgggccttgtgctggctgctttggggctgttcgttcaccatcgggacagcccactggttcaggcctcgggggggcccctggcctgctttggcctggtgtgcctgggcctggtctgcctcagcgtcctcctgttccctggccagcccagccctgcccgatgcctggcccagcagcccttgtcccacctcccgctcacgggctgcctgagcacactcttcctgcaggcggccgagatcttcgtggagtcagaactgcctctgagctgggcagaccggctgagtggctgcctgcgggggccctgggcctggctggtggtgctgctggccatgctggtggaggtcgcactgtgcacctggtacctggtggccttcccgccggaggtggtgacggactggcacatgctgcccacggaggcgctggtgcactgccgcacacgctcctgggtcagcttcggcctagcgcacgccaccaatgccacgctggcctttctctgcttcctgggcactttcctggtgcggagccagccgggccgctacaaccgtgcccgtggcctcacctttgccatgctggcctacttcatcacctgggtctcctttgtgcccctcctggccaatgtgcaggtggtcctcaggcccgccgtgcagatgggcgccctcctgctctgtgtcctgggcatcctggctgccttccacctgcccaggtgttacctgctcatgcggcagccagggctcaacacccccgagttcttcctgggagggggccctggggatgcccaaggccagaatgacgggaacacaggaaatcaggggaaacatgagtga

SEQ ID NO: 64 - protein, 852 aa, Homo sapiens
MLGPAVLGLSLWALLHPGTGAPLCLSQQLRMKGDYVLGGLFPLGEAEEAGLRSRTRPSSPVCTRFSSNGLLWALAMKMAVEEINNKSDLLPGLRLGYDLFDTCSEPVVAMKPSLMFLAKAGSRDIAAYCNYTQYQPRVLAVIGPHSSELAMVTGKFFSFFLMPQVSYGASMELLSARETFPSFFRTVPSDRVQLTAAAELLQEFGWNWVAALGSDDEYGRQGLSIFSALAAARGICIAHEGLVPLPRADDSRLGKVQDVLHQVNQSSVQVVLLFASVHAAHALFNYSISSRLSPKVWVASEAWLTSDLVMGLPGMAQMGTVLGFLQRGAQLHEFPQYVKTHLALATDPAFCSALGEREQGLEEDVVGQRCPQCDCITLQNVSAGLNHHQTFSVYAAVYSVAQALHNTLQCNASGCPAQDPVKPWQLLENMYNLTFHVGGLPLRFDSSGNVDMEYDLKLWVWQGSVPRLHDVGRFNGSLRTERLKIRWHTSDNQKPVSRCSRQCQEGQVRRVKGFHSCCYDCVDCEAGSYRQNPDDIACTFCGQDEWSPERSTRCFRRRSRFLAWGEPAVLLLLLLLSLALGLVLAALGLFVHHRDSPLVQASGGPLACFGLVCLGLVCLSVLLFPGQPSPARCLAQQPLSHLPLTGCLSTLFLQAAEIFVESELPLSWADRLSGCLRGPWAWLVVLLAMLVEVALCTWYLVAFPPEVVTDWHMLPTEALVHCRTRSWVSFGLAHATNATLAFLCFLGTFLVRSQPGRYNRARGLTFAMLAYFITWVSFVPLLANVQVVLRPAVQMGALLLCVLGILAAFHLPRCYLLMRQPGLNTPEFFLGGGPGDAQGQNDGNTGNQGKHE

SEQ ID NO: 65 - DNA, 1125 bp, Homo sapiens
  CDS (1)..(1125)
atggcccgctcgctgacctggcgctgctgcccctggtgcctgacggaggatgagaaggccgccgcccgggtggaccaggagatcaacaggatcctcttggagcagaagaagcaggaccgcggggagctgaagctgctgcttttgggcccaggcgagagcgggaagagcaccttcatcaagcagatgcggatcatccacggcgccggctactcggaggaggagcgcaagggcttccggcccctggtctaccagaacatcttcgtgtccatgcgggccatgatcgaggccatggagcggctgcagattccattcagcaggcccgagagcaagcaccacgctagcctggtcatgagccaggacccctataaagtgaccacgtttgagaagcgctacgctgcggccatgcagtggctgtggagggatgccggcatccgggcctgctatgagcgtcggcgggaattccacctgctcgattcagccgtgtactacctgtcccacctggagcgcatcaccgaggagggctacgtccccacagctcaggacgtgctccgcagccgcatgcccaccactggcatcaacgagtactgcttctccgtgcagaaaaccaacctgcggatcgtggacgtcgggggccagaagtcagagcgtaagaaatggatccattgtttcgagaacgtgatcgccctcatctacctggcctcactgagtgaatacgaccagtgcctggaggagaacaaccaggagaaccgcatgaaggagagcctcgcattgtttgggactatcctggaactaccctggttcaaaagcacatccgtcatcctctttctcaacaaaaccgacatcctggaggagaaaatccccacctcccacctggctacctatttccccagtttccagggccctaagcaggatgctgaggcagccaagaggttcatcctggacatgtacacgaggatgtacaccgggtgcgtggacggccccgagggcagcaagaagggcgcacgatcccgacgccttttcagccactacacatgtgccacagacacacagaacatccgcaaggtcttcaaggacgtgcgggactcggtgctcgcccgctacctggacgagatcaacctgctgtga

SEQ ID NO: 66 - protein, 374 aa, Homo sapiens
MARSLTWRCCPWCLTEDEKAAARVDQEINRILLEQKKQDRGELKLLLLGPGESGKSTFIKQMRIIHGAGYSEEERKGFRPLVYQNIFVSMRAMIEAMERLQIPFSRPESKHHASLVMSQDPYKVTTFEKRYAAAMQWLWRDAGIRACYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSKKGARSRRLFSHYTCATDTQNIRKVFKDVRDSVLARYLDEINLL

SEQ ID NO: 67 - DNA, 1065 bp, Rattus norvegicus
  CDS (1)..(1065)
atgggaagtggaattagttcagagagcaaggagtcagccaaaaggtccaaagaactggagaagaagcttcaggaagatgctgaacgagatgcaagaactgtgaagttgctgctattaggagcaggtgaatctggaaaaagtactattgttaaacaaatgaagatcatccacaagaatggttacagtaaacaagaatgcatggagtttaaagcagtggtttacagtaacacgttgcagtccatcctggccattgtgaaagccatgactacactagggattgattatgtcaatccgagaagtagagaggaccaacaactgcttctctccatggcaaacacactagaagatggtgacatgacgcctcagttggctgaaataattaaacgtctgtggggcgatccaggaattcaagcctgcttcgaaagggcatctgaataccagctcaatgactctgcagcttactaccttaatgacttagatagactcacagcccctgggtatgtgccaaatgaacaagacgttctacattcccgggtgaaaaccactggtatcattgaaactcaattctcctttaaagacttgaacttcagaatgtttgatgtaggtggccagagatcagaaagaaagaaatggatccactgctttgaaggagtgacgtgcattatattttgtgcagccctaagtgcctacgacatggtacttgtagaagatgaagaggtgaacagaatgcatgaaagtcttcacctcttcaacagcatctgtaatcacaagtattttgcaaccacctccattgttctgtttcttaacaagaaagatctcttccaggagaaagtgaccaaggtgcacctcagcatctgtttcccagaatacactggaccaaatacattcgaagatgcagggaactacatcaagaaccagttcctagacctgaacttaaaaaaagaagataaggaaatctattctcacatgacctgcgctactgacacacaaaacgtcaaattcgtgtttgatgccgtgacagatataataataaaagagaacctcaaagactgtgggctcttctga

SEQ ID NO: 68 - protein, 354 aa, Rattus norvegicus
MGSGISSESKESAKRSKELEKKLQEDAERDARTVKLLLLGAGESGKSTIVKQMKIIHKNGYSKQECMEFKAVVYSNTLQSILAIVKAMTTLGIDYVNPRSREDQQLLLSMANTLEDGDMTPQLAEIIKRLWGDPGIQACFERASEYQLNDSAAYYLNDLDRLTAPGYVPNEQDVLHSRVKTTGIIETQFSFKDLNFRMFDVGGQRSERKKWIHCFEGVTCIIFCAALSAYDMVLVEDEEVNRMHESLHLFNSICNHKYFATTSIVLFLNKKDLFQEKVTKVHLSICFPEYTGPNTFEDAGNYIKNQFLDLNLKKEDKEIYSHMTCATDTQNVKFVFDAVTDIIIKENLKDCGLF

SEQ ID NO: 69 - DNA, 1146 bp, Homo sapiens
  CDS (1)..(1146)
atggggtgtttgggcggcaacagcaagacgacggaagaccagggcgtcgatgaaaaagaacgacgcgaggccaacaaaaagatcgagaagcagttgcagaaagagcgcctggcttacaaggctacccaccgcctgctgctcctgggggctggtgagtctgggaaaagcactatcgtcaaacagatgaggatcctgcacgtcaatgggtttaatcccgaggaaaagaaacagaaaattctggacatccggaaaaatgttaaagatgctatcgtgacaattgtttcagcaatgagtactataatacctccagttccgctggccaaccctgaaaaccaatttcgatcagactacatcaagagcatagcccctatcactgactttgaatattcccaggaattctttgaccatgtgaaaaaactttgggacgatgaaggcgtgaaggcatgctttgagagatccaacgaataccagctgattgactgtgcacaatacttcctggaaagaatcgacagcgtcagcttggttgactacacacccacagaccaggacctcctcagatgcagagttctgacatctgggatttttgagacacgattccaagtggacaaagtaaacttccacatgtttgatgttggtggccagagggatgagaggagaaaatggatccagtgctttaacgatgtcacagctatcatttacgtcgcagcctgcagtagctacaacatggtgattcgagaagataacaacaccaacaggctgagagagtccctggatctttttgaaagcatctggaacaacaggtggttacggaccatttctatcatcttgttcttgaacaaacaagatatgctggcagaaaaagtcttggcagggaaatcaaaaattgaagactatttcccagaatatgcaaattatactgttcctgaagacgcaacaccagatgcaggagaagatcccaaagttacaagagccaagttctttatccgggacctgtttttgaggatcagcacggccaccggtgacggcaaacattactgctacccgcacttcacctgcgccgtggacacagagaacatccgcagggtgttcaacgactgccgcgacatcatccagcggatgcacctcaagcagtatgagctcttgtga

SEQ ID NO: 70 - protein, 381 aa, Homo sapiens
MGCLGGNSKTTEDQGVDEKERREANKKIEKQLQKERLAYKATHRLLLLGAGESGKSTIVKQMRILHVNGFNPEEKKQKILDIRKNVKDAIVTIVSAMSTIIPPVPLANPENQFRSDYIKSIAPITDFEYSQEFFDHVKKLWDDEGVKACFERSNEYQLIDCAQYFLERIDSVSLVDYTPTDQDLLRCRVLTSGIFETRFQVDKVNFHMFDVGGQRDERRKWIQCFNDVTAIIYVAACSSYNMVIREDNNTNRLRESLDLFESIWNNRWLRTISIILFLNKQDMLAEKVLAGKSKIEDYFPEYANYTVPEDATPDAGEDPKVTRAKFFIRDLFLRISTATGDGKHYCYPHFTCAVDTENIRRVFNDCRDIIQRMHLKQYELL

SEQ ID NO: 71 - DNA, 588 bp, Encephalomyocarditis virus
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 72 - DNA, 135 bp, Rattus norvegicus
  CDS (1)..(135)
atggccgctgttacctatccttcatccgtgcctacgaccttggaccctgggaatgcatcctcagcctggcccctggacacgtccctggggaatgcatctgctggcactagcctggcaggactggctgtcagtggc

SEQ ID NO: 73 - protein, 45 aa, Rattus norvegicus
MAAVTYPSSVPTTLDPGNASSAWPLDTSLGNASAGTSLAGLAVSG

SEQ ID NO: 74 - DNA, 1047 bp, Homo sapiens
  CDS (1)..(1047)
atgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaa

SEQ ID NO: 75 - protein, 348 aa, Homo sapiens [sequence omitted]

SEQ ID NO: 76 - DNA, 45 bp, Artificial Sequence
  V5 epitope
ggtaagcctatccctaaccctctcctcggtctcgattctacgtga

SEQ ID NO: 77 - DNA, 1125 bp, Artificial Sequence
  coding Garufa16/Garufagustducin chimera protein
atggcccgctcgctgacctggcgctgctgcccctggtgcctgacggaggatgagaaggccgccgcccgggtggaccaggagatcaacaggatcctcttggagcagaagaagcaggaccgcggggagctgaagctgctgcttttgggcccaggcgagagcgggaagagcaccttcatcaagcagatgcggatcatccacggcgccggctactcggaggaggagcgcaagggcttccggcccctggtctaccagaacatcttcgtgtccatgcgggccatgatcgaggccatggagcggctgcagattccattcagcaggcccgagagcaagcaccacgctagcctggtcatgagccaggacccctataaagtgaccacgtttgagaagcgctacgctgcggccatgcagtggctgtggagggatgccggcatccgggcctgctatgagcgtcggcgggaattccacctgctcgattcagccgtgtactacctgtcccacctggagcgcatcaccgaggagggctacgtccccacagctcaggacgtgctccgcagccgcatgcccaccactggcatcaacgagtactgcttctccgtgcagaaaaccaacctgcggatcgtggacgtcgggggccagaagtcagagcgtaagaaatggatccattgtttcgagaacgtgatcgccctcatctacctggcctcactgagtgaatacgaccagtgcctggaggagaacaaccaggagaaccgcatgaaggagagcctcgcattgtttgggactatcctggaactaccctggttcaaaagcacatccgtcatcctctttctcaacaaaaccgacatcctggaggagaaaatccccacctcccacctggctacctatttccccagtttccagggccctaagcaggatgctgaggcagccaagaggttcatcctggacatgtacacgaggatgtacaccgggtgcgtggacggccccgagggcagcaagttaaaaaaagaagataaggaaatctattctcacatgacctgcgctactgacacacaaaacgtcaaattcgtgtttgatgccgtgacagatataataataaaagagaacctcaaagactgtgggctcttctga

SEQ ID NO: 78 - DNA, 720 bp, Homo sapiens
  CDS (1)..(720)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 79 - protein, 239 aa, Homo sapiens
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 80 - DNA, 5529 bp, Artificial Sequence
  pcDNA3.1 Hygro(+)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 81 - DNA, 6069 bp, Artificial Sequence
  pEAK10 vector
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctgaagccagttaccagtggctgctgccagtggcgataagtcgtgtcttaccgggttggactcaagagatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattgatgccgggcaagagcaactcggtcgccgcatacactattctcagaatgacttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcggcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagacgatctgcacactggtatttcggtttttggggccgcgggcggcgacggggcccgtgcgtcccagcgcacatgcatgttcggcgaggcggggcctgcgagcgcggccaccgagaatcggacgggggtagtctcaagctggccggcctgctctggtgcctggcctcgcgccgccgtgtatcgccccgccctgggcggcaaggctgggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagcttgaattcggcgcgccgcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggtcgtggaaggtgctactccagtgcccaccagccttgtcctaataaaattaagttgcatcattttgtttgactaggtgtccttgtataatattatggggtggaggcgggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

(19) United States
(12) Patent Application Publication
(10) Pub. No.: US 2020/0247859 A1
(43) Pub. Date: Aug. 6, 2020

(54) CONSTRUCT FOR EXPRESSING G PROTEIN-COUPLED RECEPTOR OR SUBUNIT THEREOF, AND USE OF SAID CONSTRUCT

(71) Applicant: JAPAN TOBACCO INC., Tokyo (JP)

(72) Inventors: Ippei SHIMOMURA, Tokyo (JP); Takeshi YAMAMOTO, Tokyo (JP); Masafumi JOTAKI, Tokyo (JP); Naoya TOBITA, Tokyo (JP)

(73) Assignee: JAPAN TOBACCO INC., Tokyo (JP)

(21) Appl. No.: 16/855,849

(22) Filed: Apr. 22, 2020

Related U.S. Application Data
(63) Continuation of application No. PCT/JP2018/038391, filed on Oct. 16, 2018.

(30) Foreign Application Priority Data
Oct. 23, 2017 (JP) 2017-204687

(51) Int. Cl. C07K 14/47; C12N 15/63; G01N 33/50

(52) U.S. Cl. CPC C07K 14/4722; G01N 33/5041; C12N 15/63

(57) ABSTRACT

An purpose of the present invention is to provide a construct for expressing a G protein-coupled receptor, and the use of said construct. The construct is used for expressing a G protein-coupled receptor protein or a subunit thereof, wherein the construct is characterized by including a nucleic acid that encodes G protein α subunit protein; a nucleic acid that comprises an IRES sequence; and a nucleic acid that encodes the G protein-coupled receptor protein or a subunit thereof in the stated order from the 5′ side in a single vector.

### CROSS REFERENCE TO RELATED APPLICATIONS

The present application is a continuation application of International Application No. PCT/JP2018/038391, filed on Oct. 16, 2018.

### TECHNICAL FIELD

The present invention relates to a construct for expressing a G protein-coupled receptor or a subunit thereof, transformed cells prepared by gene transfer of the construct of the present invention, a method for measuring a physiological response to a substance using the transformed cells of the present invention, a use of the transformed cells of the present invention for measuring a physiological response to a substance, and a kit for use in a method for measuring a physiological response to a substance, which comprises the transformed cells of the present invention.

### BACKGROUND ART

### 1. G Protein-Coupled Receptor and G Protein-Mediated Signaling

G protein-coupled receptors are a form of receptors present on eukaryotic cytoplasmic membrane or intracellular constitutive membrane. When various signals (neurotransmitters, hormones, chemical substances, light, etc.) are received from outside the cells (outside the membrane), G protein-coupled receptors undergo a structural change to activate trimeric G proteins bound to the inner side of the membrane for signaling.

G proteins are heterotrimers bound to the inner surface of cell membranes, in which Gβγ dimers are tightly bound to Gα subunits. When activated upon binding of ligands to G protein-coupled receptors, G proteins bound to the G protein-coupled receptors dissociate their own GDP from Gα subunits and bind to new GTP molecules (Conversion from GDP type→GTP type). This conversion dissociates Gα subunits, Gβγ dimers and G protein-coupled receptors, respectively. The thus dissociated Gα subunits activate effector proteins downstream thereof.

G protein-coupled receptors and G proteins downstream thereof control many basic physiochemical reactions in intracellular signal networks such as olfactory sense, gustatory sense, vision, neurotransmission, metabolism, cell differentiation and cell proliferation, and inflammatory response and immune response.

### 2. Findings on Gustatory Sense and Olfactory Sense

Gustatory sense is sensation that especially results from binding of a substance to specific receptors present on the surface of the tongue, when the substance is in the mouth. The mammalian gustatory sense is composed of five basic tastes, that is, salty taste, sour taste, sweet taste, umami taste and bitter taste, and is considered to be formed by integration of these basic tastes. At present, salty taste and sour taste are said to be sensed via several ionotropic receptors expressed on the cell membrane on the proximal side of taste cells present in taste buds on the surface of the tongue.

Sweet taste, umami taste and bitter taste are considered to be sensed by signaling via G protein-coupled receptors present in taste cells and G proteins coupled thereto. Specifically, it has been revealed that bitter taste is accepted by molecules (bitter taste receptors) (25 types in human) designated as the T2R family, sweet taste is accepted by a T1R2+T1R3 heterodimer (sweet taste receptor), and umami taste is accepted by a T1R1+T1R3 heterodimer (umami taste receptor).

The signal transduction mechanism of gustatory sense information is generally understood as follows. Specifically, it is commonly considered that: first, a taste substance is bound at receptors of taste cells to activate a signal transduction process through mediation by intracellular second messengers (IP3, DAG) and the like, increasing the intracellular calcium concentration; calcium ions supplied into the cells cause synapses to release neurotransmitters, so as to cause neurons to generate an action potential; gustatory sense signals originating from the receptors are transmitted from the gustatory nerve to the brain; and thus the gustatory sense information is identified and determined.

Olfactory sense receptors are also G protein-coupled receptors, as are gustatory sense receptors such as bitter taste receptors, sweet taste receptors and umami taste receptors. Olfactory sense receptors are present on olfactory cells of the olfactory epithelium. The location of olfactory cells is the only place in the human body where neurons are in direct contact with the outside world, and the other end thereof is directly connected to a region called the olfactory bulb of the brain. “Odorant” is a low molecular weight compound having a molecular weight of about 30 to 300, and hundreds of thousands of odorants are said to exist on the earth. As many as 400 or more olfactory sense receptors have been identified in humans. When an odorant is bound at an olfactory sense receptor, in a manner similar to that of gustatory sense receptors, such as bitter taste receptors, sweet taste receptors, and umami taste receptors, intracellular calcium concentration increases through the signal transduction process mediated by intracellular second messengers, etc.

3. Evaluation of G Protein-Coupled Receptor Agonists

Chemical substances, neurotransmitters, hormones, etc., contained in foods and beverages, etc., can be G protein-coupled receptor agonists in a series of signaling, whereby their binding at G protein-coupled receptors causes a structural change to activate G proteins bound to the inside of the membrane. It is desirable to evaluate conveniently and efficiently the physiological responses to these candidate G protein-coupled receptor agonists. If it is possible to appropriately evaluate the physiological responses to such candidate protein-coupled receptor agonists, beneficial effects such as improvement of the taste, quality, etc., of foods and beverages, medicines, and the like, and regulation of the dosages of protein-coupled receptor agonists, can be achieved.

On the other hand, substances contained in the above foods, beverages and the like have also been evaluated by sensory tests. However, the evaluation by such sensory tests is known to be intrinsically problematic in that ensuring the objectivity of the evaluation is not easy, such evaluation is burdensome in terms of human factors, time and money, and a large amount of substances cannot be evaluated in a short time, for example.

Therefore, as an evaluation method that is an alternative to the sensory tests, a method that involves preparing a cell line expressing a G protein-coupled receptor and a Gα protein, directly adding a G protein-coupled receptor agonist (candidate compound) to such a cell line, and then evaluating the effects has been reported.

In cell lines for evaluation of G protein-coupled receptor agonists, a G protein-coupled receptor protein and a Gα protein are required to be expressed in the same cell. In order to express the two protein types, a double-gene transfer method is generally widely used in which two vector types comprising nucleic acids encoding respective proteins are introduced simultaneously or sequentially. This is an embodiment as follows, for example:

(a) cotransfection of host cells with a vector comprising a nucleic acid encoding a Gα protein and a vector comprising a nucleic acid encoding the G protein-coupled receptor protein or a subunit thereof and

(b) transformation of a cell line stably expressing a Gα protein with a vector comprising a nucleic acid encoding the G protein-coupled receptor protein or a subunit thereof.

Examples of using such double-gene transfer method for bitter taste receptors are described in Chem. Senses 35, p. 157-170, 2010; The Journal of Neuroscience, 23(19), p. 7376-7380, 2003; Biochem Biophys Res Commun, 365(4): p. 851-855, 2008. Further, examples of the same for sweet taste receptors are described in Japanese Unexamined Patent Publication No. 2008-13570, Japanese Unexamined Patent Publication No. 2008-228690, and WO2007-121604.

However, in any of these cases, the preparation difficulty is high (the success rate is low), and stable data cannot be acquired. Therefore, these cell lines cannot be said as having sufficient quality for use in evaluation of G protein-coupled receptor agonists.

As a method for expressing two protein types, a method in which a co-expression vector prepared by insertion of nucleic acids expressing two protein types, respectively, into one vector is known in addition to the double-gene transfer method. In general, even if two nucleic acid types expressing two protein types are directly inserted in tandem into a single vector, the more distant the nucleic acid located from a promoter, the more difficult it is to be translated, resulting in the decreased protein expression level. Therefore, ingenuity is required for increasing the translational efficiency of nucleic acids located more distant from a promoter.

Japanese Patent No. 5905187 describes “a sweet taste receptor expression construct obtained by insertion of the genes encoding gustatory receptor subunits T1R2 and T1R3 and a G protein α subunit, respectively, into the same plasmid, wherein a gene encoding the G protein α subunit is linked via an IRES sequence to a site immediately following the gene encoding the sweet taste receptor subunit T1R2 located downstream of an EF-1α promoter, and, a gene encoding the G protein α subunit is linked via an IRES sequence to a site immediately following the gene encoding the sweet taste receptor subunit T1R3 located downstream of a CMV promoter located downstream of the above-mentioned G protein α subunit-encoding gene”. The construct of Japanese Patent No. 5905187 has a specific structure comprising, from 5′ in this order, EF-1α promoter-T1R2-IRES-Gα-CMV promoter-TIR3-IRES-Gα. In this construct, T1R2 and T1R3 are located downstream of particular promoters, respectively, and located upstream of the IRESs, respectively.

IRES is an RNA factor that enables cap-independent translational initiation to further increase the efficiency of protein synthesis. It is known that IRES-mediated translation tends to be less efficient compared to that of upstream proteins that generally undergo cap-dependent translation.

Prior to the present invention, cell lines having sufficient quality for use in evaluation of G protein-coupled receptor agonists have not been obtained.

### CITATION LIST

### Patent Literature

PTL 1: Japanese Unexamined Patent Publication No. 2008-13570
PTL 2: Japanese Unexamined Patent Publication No. 2008-228690
PTL 3: WO2007-121604

### Non Patent Literature

NPL1: Chem. Senses 35, p. 157-170, 2010;
NPL2: The Journal of Neuroscience, 23(19), p. 7376-7380, 2003;
NPL3: Biochem Biophys Res Commun, 365(4): p. 851-855, 2008
NPL4: Molecular BioSystems, 7, p. 911-919, 2011
NPL5: Proc. Natl. Acad. Sci. USA, Vol. 88, p. 5587-5591, 1991
NPL6: Journal of Virology, Vol. 86, No. 3, p. 1468-1486, 2012
NPL7: GENES & DEVELOPMENT, Vol, 15, p. 1593-1612, 20
NPL8: Archives of Physiology and Biochemistry Vol. 110 p. 137-45, 2002
NPL9: Nature Genetics, Vol. 32, p. 397-401, 2002;
NPL10: Cell, Vol. 100, p. 703-711, 2000

### SUMMARY OF INVENTION

### Technical Problem

As a result of intensive efforts to solve the above problems, the present inventors have discovered that a construct for expressing a G protein-coupled receptor protein or a subunit thereof comprising: a nucleic acid encoding a G protein α subunit protein; a nucleic acid comprising an IRES sequence; and a nucleic acid encoding the G protein-coupled receptor protein or a subunit thereof in the stated order from the 5′ side in a single vector, provides high efficiency expression of the G protein-coupled receptor protein or a subunit thereof and high efficiency response to a G protein-coupled receptor agonist. Hence, the present inventors have conceived of the present invention.

The expression level of a protein encoded by a gene placed on the 3′ side of IRES is thought to be low. Accordingly, in the present invention, the fact that a G protein-coupled receptor protein or a subunit thereof is expressed with high efficiency is contrary to conventional knowledges and is characteristic.

### Solution to Problem

The present invention encompasses, but is not limited to, the following embodiments.

### Embodiment 1

A construct for expressing a G protein-coupled receptor protein or a subunit thereof, comprising: a nucleic acid encoding a G protein α subunit protein; a nucleic acid comprising an IRES sequence; and a nucleic acid encoding the G protein-coupled receptor protein or a subunit thereof in the stated order from the 5′ side in a single vector.

### Embodiment 2

The construct according to embodiment 1, wherein the G protein-coupled receptor or a subunit thereof is a G protein-coupled receptor of a rhodopsin-like receptor class or a metabotropic glutamate receptor/metabotropic pheromone receptor class.

### Embodiment 3

The construct according to embodiment 1, wherein the G protein-coupled receptor is a G protein-coupled receptor of the rhodopsin-like receptor class.

### Embodiment 4

The construct according to embodiment 1, wherein the G protein-coupled receptor is a bitter taste receptor.

### Embodiment 5

The construct according to any one of embodiments 1 to 4, wherein the vector is a plasmid vector.

### Embodiment 6

The construct according to any one of embodiments 1 to 4, wherein the vector is a plasmid vector comprising a CMV promoter.

### Embodiment 7

The construct according to any one of embodiments 1 to 4, wherein the vector is a plasmid vector comprising a CMV promoter and a hygromycin resistance gene.

### Embodiment 8

The construct according to any one of embodiments 1 to 4, wherein the vector is a plasmid vector comprising an EF1α promoter.

### Embodiment 9

The construct according to any one of embodiments 1 to 4, wherein the vector is a plasmid vector comprising an EF1α promoter and a puromycin resistance gene.

### Embodiment 10

The construct according to any one of embodiments 1 to 9, wherein the IRES sequence is a type 2 IRES sequence.

### Embodiment 11

The construct according to any one of embodiments 1 to 10, wherein the IRES sequence is an IRES sequence derived from the encephalomyocarditis virus.

### Embodiment 12

The construct according to any one of embodiments 1 to 7, comprising a nucleic acid encoding a polypeptide having a function of enhancing the translocation of the G protein-coupled receptor protein or a subunit thereof to a cell membrane, 5′ to the nucleic acid encoding the G protein-coupled receptor protein or a subunit thereof.

### Embodiment 13

The construct according to embodiment 12, wherein the polypeptide having a function of enhancing the translocation of the G protein-coupled receptor protein or a subunit thereof to a cell membrane is a polypeptide comprising N-terminal 45 amino acids of a rat somatostatin receptor or a polypeptide comprising N-terminal 39 amino acids of bovine rhodopsin.

### Embodiment 14

The construct according to any one of embodiments 1 to 13, wherein the G protein α subunit protein is a G protein α subunit protein of the Gq family, a G protein α subunit protein of the Gi family, a G protein α subunit protein of the Gs family, or a chimeric protein thereof.

### Embodiment 15

The construct according to any one of embodiments 1 to 14, wherein the G protein α subunit protein is Gα16, Gαgust or Gαolf, or a chimeric protein thereof.

### Embodiment 16

The construct according to any one of embodiments 1 to 15, wherein the G protein α subunit protein is human Gα16/rat Gαgust.

### Embodiment 17

The construct according to embodiment 16, wherein the G protein α subunit protein is human Gα16/rat Gαgust43.

### Embodiment 18

The construct according to any one of embodiments 1 to 17, wherein the G protein-coupled receptor protein is a human G protein-coupled receptor protein.

### Embodiment 19

Transformed cells, into which the construct according to any one of embodiments 1 to 18 is introduced.

### Embodiment 20

A method for measuring a physiological response to a substance, comprising adding a substance to the transformed cells according to embodiment 19, and then measuring a physiological response induced by the substance.

### Embodiment 21

The measurement method according to embodiment 20, wherein the substance is a bitter substance, an odorant, an umami substance or a sweet substance, or a modulator for these substances.

### Embodiment 22

Use of the transformed cells according to embodiment 19, for measurement of a physiological response to a substance.

### Embodiment 23

A kit comprising the transformed cells according to embodiment 19, which is used for a method for measuring a physiological response to a substance by adding a substance to the transformed cells, and then measuring a physiological response induced by the substance.

### Advantageous Effects of Invention

Cells transformed with the construct of the present invention express a G protein-coupled receptor protein or a subunit thereof with high efficiency and respond with high efficiency to a G protein-coupled receptor agonist. The transformed cells of the present invention provide cell lines having sufficient quality to be used for evaluation of G protein-coupled receptor agonists.

### BRIEF DESCRIPTION OF DRAWINGS

FIG. 1 illustrates the construction of a construct prepared in Example 1. In FIG. 1, the term “CMV” refers to a CMV promoter.

FIG. 2 depicts the results of measuring the response of a T2R16 stable expression cell line to a bitter substance, salicin.

FIG. 3 depicts the results of measuring the response of a T2R40 stable expression cell line to a bitter substance, diphenydol, and the response of a T2R43 stable expression cell line to a bitter substance, quinine.

FIG. 4 depicts the results of measuring the response of T2R16 transiently expressing cells to the bitter substance, salicin.

FIG. 5 depicts the results of measuring the response to a T2R-independent Gα16 agonist (isoproterenol) in T2R16 transiently expressing cells.

FIG. 6 depicts the results of measuring the expression level of T2R16 in T2R16 transiently expressing cells.

FIG. 7 depicts the results of comparing response intensity before and the same after cryopreservation of the T2R16 stable expression cell line.

FIG. 8 depicts the results of examining the correlation between the response intensity of a Gα-T2R40 stable expression cell line to a T2R40 agonist and the response intensity of the same to a Gα agonist.

FIG. 9 depicts the results of measuring the expression level of T1R2 in a cell line (Gα-T1R2+T1R3) transiently expressing Gα-T1R2 and T1R3 after double-gene transfer thereof.

FIG. 10 depicts the results of measuring the response to a T2R-independent Gα16 agonist (isoproterenol) in a cell line (Gα-T1R2+T1R3) transiently expressing Gα-T1R2 and T1R3 after double-gene transfer thereof.

FIG. 11 depicts the results of measuring the response to a sweetener, aspartame, in a cell line (Gα-T1R2+T1R3) transiently expressing Gα-T1R2 and T1R3 after double-gene transfer thereof.

FIG. 12 depicts the results of measuring the expression level of T1R3 in a cell line (Gα-T1R3+T1R2) transiently expressing Gα-T1R3 and T1R2 after double-gene transfer thereof.

FIG. 13 depicts the results of measuring the response to a T2R-independent Gα16 agonist (isoproterenol) in a cell line (Gα-T1R3+T1R2) transiently expressing Gα-T1R3 and T1R2 after double-gene transfer thereof.

FIG. 14 depicts the results of measuring the response to a sweetener, cyclamate, in a cell line (Gα-T1R3+T1R2) transiently expressing Gα-T1R3 and T1R2 after double-gene transfer thereof.

FIG. 15 depicts the results of measuring the expression level of EmGFP when a green fluorescent protein (EmGFP) was expressed upstream and downstream of IRES.

FIG. 16 depicts the results of examining the expression level of T2R16 in HEK293T cells transiently expressing pEAK10-T2R16-Gα and pEAK10-Gα-T2R16.

FIG. 17 depicts the results of examining seven T2R subtypes (bitter taste receptors) for the response of transiently expressing cells to each agonist (bitter substance).

FIG. 18 depicts the results of examining a T2R38 subtype (bitter taste receptor) for the response of stably expressing cells to an agonist (6-n-propylthiouracil). Black bars indicate the result of T2R38, and diagonally shaded bars indicate the results of a control (a cell line (HEK293T) not incorporating the expression vector Gα-T2R38).

FIG. 19 depicts the results of examining the expression levels of fourteen T2R subtypes (bitter taste receptors) in transiently expressing cells (compared to T2R4).

FIG. 20 depicts the results of examining the response in cells transiently expressing an olfactory receptor (OR3A1). The diagonally shaded bars indicate the results of introducing OR3A1-Gαolf and black bars indicate the results of introducing Gαolf-OR3A1. The dotted bars (OR3A1+Gαolf) indicate the results of introducing OR3A1 and Gαolf genes separately into pcDNA for double-gene transfer thereof into HEK293T.

FIG. 21 depicts the expression levels of OR3A1 and Gαolf in cells transiently expressing the olfactory receptor (OR3A1). The diagonally shaded bars indicate the results of OR3A1, and the black bars indicate the results of Gαolf “OR3A1+Gαolf” indicates the result of introducing the genes of OR3A1 and Gαolf separately into pcDNA for double-gene transfer thereof into HEK293T.

### DESCRIPTION OF EMBODIMENTS

The present invention relates to a construct for expressing a G protein-coupled receptor or a subunit thereof, transformed cells containing the construct of the present invention through gene transfer, a method for measuring a physiological response to a substance using the transformed cells of the present invention, a use of the transformed cells of the present invention for measuring a physiological response to a substance, and a kit comprising the transformed cells of the present invention, which is used for a method for measuring a physiological response to a substance. Hereinafter, embodiments of the present invention will be described without limitation.

I. Construct for Expressing G Protein-Coupled Receptor or Subunit Thereof

The present invention relates to, in one embodiment, a construct for expressing a G protein-coupled receptor or a subunit thereof. The construct of the present invention comprises: a nucleic acid encoding a G protein α subunit protein; a nucleic acid comprising an IRES sequence; and a nucleic acid encoding the G protein-coupled receptor protein or a subunit thereof in the stated order from the 5′ side in a single vector.

(1) G Protein-Coupled Receptor or Subunit Thereof

“G protein-coupled receptors (GPCR)” are a form of receptors present on eukaryotic cytoplasmic membrane or intracellular constitutive membrane. Seven helical structures penetrate the cell membrane (or intracellular constituent membrane), and the N-terminus is located outside the membrane, and the C-terminus is located inside the membrane. Because of such a characteristic structure, these receptors are also referred to as another name “7-transmembrane receptors”. The extramembrane loop contains two well-conserved cysteine residues, and the receptor structure is stabilized by disulfide bonds. When G protein-coupled receptors accept various signals (neurotransmitters, hormones, chemical substances, light, etc.) from outside the cells (outside the membrane), the receptors undergo a structural change to activate trimeric G proteins bound to the inner side of the membrane, so that signaling is performed. G protein-coupled receptors control many basic physiochemical reactions in intracellular signal networks such as olfactory sense, gustatory sense, vision, neurotransmission, metabolism, cell differentiation and cell proliferation, and inflammatory responses and immune responses.

G protein-coupled receptors are classified into six classes based on similarities in amino acid sequences, pharmacological dynamics, functions, etc. (Molecular BioSystems, 7, p. 911-919, 2011).

Class A: rhodopsin-like receptor

Class B: secretin receptor family

Class C: metabotropic glutamate receptor/metabotropic pheromone receptor

Class D: fungal mating factor receptor

Class E: cyclic AMP (cAMP) receptor

Class F: frizzled, smoothened

(i) Class A: Rhodopsin-Like Receptor

The rhodopsin-like receptors of class A have a relatively short N-terminal extracellular domain, wherein a plurality of transmembrane domains form a ligand binding site. Class A rhodopsin-like receptors account for 80% or more of G protein-coupled receptors, and include bitter taste receptors, olfactory sense receptors, rhodopsin, adrenoceptors, muscarinic acetylcholine receptors and the like. An amino acid sequence conserved in the rhodopsin-like receptor is the E/DRY (Asp/Glu, Arg, Tyr) motif located inside the membrane of the third transmembrane domain.

Bitter taste receptors are G protein-coupled receptors belonging to rhodopsin-like receptors, and have been identified as members of the T2R family on human and mouse chromosome 6. At least 35 types of T2R in mice and 25 types of T2R in humans function as bitter taste receptors. Examples of the relationship between human T2R and ligands are as follows.

T2R4: denatonium benzoate;

T2R10: strychnine;

T2R14: nonspecific binding of bitter substances with many different structural features

T2R16: β-glucopyranoside (salicin)

T2R31: aristolochic acid

T2R38: bitter substances having isothiocyanate groups, for example, phenylthiocarbamide (PTC) and propylthiouracil (PROP)

T2R40: diphenydol

T2R43: quinine

T2R46: absinthin, strychnine, denatonium

The amino acid sequences of the proteins of and the nucleic acid sequences of the genes of mouse T2R and human T2R are known. For example, the amino acid sequences and the gene sequences of 25 types of human T2R bitter taste receptors are registered in the National Center for Biotechnology Information (NCBI) or GenBank under the accession numbers described in Table 1 below.

TABLE 1

| | | | Another | | | | Difference between |
|---|---|---|---|---|---|---|---|
| | Amino | | name | | | | a sequence used |
| Nucleotide | acid | Gene | (former | Data | ACCESSION | | in Example and |
| sequence | sequence | name | name) | source | No. | Title | a sequence in DB |
| 1 | 2 | hTAS2R1 | | NCBI | NM_019599.2 | Homo sapiens taste 2 | Regarding those |
| | | | | Reference | | receptor member 1 | subjected to |
| | | | | Sequence | | (TAS2R1), mRNA | insertion of V5, |
| 3 | 4 | hTAS2R3 | | GenBank | AF227130.1 | Homo sapiens candidate | the 3′ terminal |
| | | | | | | taste receptor T2R3 | stop codon, a |
| | | | | | | gene, complete cds | nucleotide |
| 5 | 6 | hTAS2R4 | | NCBI | NM016944 | Homo sapiens taste 2 | triplet, was |
| | | | | Reference | | receptor member 4 | deleted and V5 |
| | | | | Sequence | | (TAS2R4), mRNA | (including a |
| 7 | 8 | hTAS2R5 | | GenBank | BC101588.1 | Homo sapiens taste | stop codon) was |
| | | | | | | receptor, type 2, | inserted. |
| | | | | | | member 5, mRNA (cDNA | |
| | | | | | | clone MGC: 126637 | |
| | | | | | | IMAGE: 8069094), | |
| | | | | | | complete cds | |
| 9 | 10 | hTAS2R7 | | NCBI | NM_023919.2 | Homo sapiens taste 2 | |
| | | | | Reference | | receptor member 7 | |
| | | | | Sequence | | (TAS2R7), mRNA | |
| 11 | 12 | hTAS2R8 | | NCBI | NM_023918.1 | Homo sapiens taste 2 | |
| | | | | Reference | | receptor member 8 | |
| | | | | Sequence | | (TAS2R8), mRNA | |
| 13 | 14 | hTAS2R9 | | NCBI | NM_023917.2 | Homo sapiens taste 2 | |
| | | | | Reference | | receptor member 9 | |
| | | | | Sequence | | (TAS2R9), mRNA | |
| 15 | 16 | hTAS2R10 | | NCBI | NM_023921.1 | Homo sapiens taste 2 | |
| | | | | Reference | | receptor member 10 | |
| | | | | Sequence | | (TAS2R10), mRNA | |
| 17 | 18 | hTAS2R13 | | NCBI | NM_023920.2 | Homo sapiens taste 2 | |
| | | | | Reference | | receptor member 13 | |
| | | | | Sequence | | (TAS2R13), mRNA | |
| 19 | 20 | hTAS2R14 | | NCBI | NM_023922.1 | Homo sapiens taste 2 | |
| | | | | Reference | | receptor member 14 | |
| | | | | Sequence | | (TAS2R14), mRNA | |
| 21 | 22 | hTAS2R16 | | GenBank | AF227139.1 | Homo sapiens candidate | |
| | | | | | | taste receptor T2R16 | |
| | | | | | | gene, complete cds | |
| 23 | 24 | hTAS2R38 | | GenBank | AY258597.1 | Homo sapiens PTC bitter | |
| | | | | | | taste receptor (PTC) | |
| | | | | | | gene, PTC-taster allele, | |
| | | | | | | complete cds | |
| 25 | 26 | hTAS2R39 | | NCBI | NM_176881.2 | Homo sapiens taste 2 | |
| | | | | Reference | | receptor member 39 | |
| | | | | Sequence | | (TAS2R39), mRNA | |
| 27 | 28 | hTAS2R40 | | NCBI | NM_176882.1 | Homo sapiens taste 2 | |
| | | | | Reference | | receptor member 40 | |
| | | | | Sequence | | (TAS2R40), mRNA | |
| 29 | 30 | hTAS2R41 | | GenBank | AF494232.1 | Homo sapiens candidate | |
| | | | | | | taste receptor TAS2R41 | |
| | | | | | | gene, complete cds | |
| 31 | 32 | hTAS2R42 | | NCBI | NM_181429.2 | Homo sapiens taste 2 | |
| | | | | Reference | | receptor member 42 | |
| | | | | Sequence | | (TAS2R42), mRNA | |
| 33 | 34 | hTAS2R43 | | GenBank | BC117423.1 | Homo sapiens taste | |
| | | | | | | receptor, type 2, | |
| | | | | | | member 43, mRNA (cDNA | |
| | | | | | | clone MGC: 151032 | |
| | | | | | | IMAGE: 40125974), | |
| | | | | | | complete cds | |
| 35 | 36 | hTAS2R44 | hTAS2R31 | GenBank | AF494228 | Homo sapiens candidate | Regarding those |
| | | | | | | taste receptor TAS2R44 | subjected to |
| | | | | | | gene, complete cds | insertion of V5, |
| 37 | 38 | hTAS2R45 | | GenBank | AF494226.1 | Homo sapiens candidate | the 3′ terminal |
| | | | | | | taste receptor TAS2R45 | stop codon, a |
| | | | | | | gene, complete cds | nucleotide |
| 39 | 40 | hTAS2R46 | | NCBI | NM_176887.2 | Homo sapiens taste 2 | triplet, was |
| | | | | Reference | | receptor member 46 | deleted and V5 |
| | | | | Sequence | | (TAS2R46), mRNA | (including a |
| 41 | 42 | hTAS2R47 | hTAS2R30 | NCBI | NM_001097643.1 | Homo sapiens taste 2 | stop codon) was |
| | | | | Reference | | receptor member 30 | inserted. |
| | | | | Sequence | | (TAS2R30), mRNA | |
| 43 | 44 | hTAS2R48 | hTAS2R19 | NCBI | NM_176888.2 | Homo sapiens taste 2 | |
| | | | | Reference | | receptor member 19 | |
| | | | | Sequence | | (TAS2R19), mRNA | |
| 45 | 46 | hTAS2R49 | hTAS2R20 | NCBI | NM_176889.3 | Homo sapiens taste 2 | |
| | | | | Reference | | receptor member 20 | |
| | | | | Sequence | | (TAS2R20), mRNA | |
| 47 | 48 | hTAS2R50 | | NCBI | NM_176890.2 | Homo sapiens taste 2 | |
| | | | | Reference | | receptor member 50 | |
| | | | | Sequence | | (TAS2R50), mRNA | |
| 49 | 50 | hTAS2R60 | | NCBI | NM_177437.1 | Homo sapiens taste 2 | |
| | | | | Reference | | receptor member 60 | |
| | | | | Sequence | | (TAS2R60), mRNA | |

In the description, the term “gene” is used synonymously with “nucleic acid” unless otherwise specified, and the present invention includes an embodiment in which a control sequence such as a promoter is not contained. Nucleic acids also include DNA (including both genomic DNA and cDNA), RNA (mRNA), and chimeras of DNA and RNA. The term “gene sequence” of a particular protein refers to a DNA sequence (containing no intron) encoding the amino acid sequence of the protein.

Olfactory sense receptors are also G protein-coupled receptors belonging to rhodopsin-like receptors as in the case of bitter taste receptors. Olfactory sense receptors have an average length of about 310 amino acid residues and share several motif sequences. A particularly characteristic motif sequence is MAYDRYVAIC which is present from the third transmembrane site to the fourth intramembrane loop. Among these motif sequences, three amino acid residues of DRY (Asp/Glu, Arg, Tyr) are common with respect to other rhodopsin-like receptors and are considered to be important for G protein activation. The amino acid of the proteins and the nucleic acid sequences of the genes of many olfactory sense receptors are known.

For example, the amino acid sequences and the gene sequences of human olfactory sense receptors are registered in the NCBI or the GenBank under the accession numbers described in Table 2 below.

TABLE 2

| | | | Another | | | | Difference between |
|---|---|---|---|---|---|---|---|
| | Amino | | name | | | | a sequence used |
| Nucleotide | acid | Gene | (former | Data | ACCESSION | | in Example and |
| sequence | sequence | name | name) | source | No. | Title | a sequence in DB |
| 51 | 52 | hOR3A1 | | NCBI | NM_002550.2 | Homo sapiens olfactory | A start codon, |
| | | | | Reference | | receptor family 3 | a nucleotide |
| | | | | Sequence | | subfamily A member 1 | triplet, was |
| | | | | | | (OR3A1), mRNA | deleted and the |
| 53 | 54 | hOR1D2 | | NCBI | NM_002548.2 | Homo sapiens olfactory | 5′ terminal 63 |
| | | | | Reference | | receptor family 1 | nucleotides of |
| | | | | Sequence | | subfamily D member 2 | Rho (including |
| | | | | | | (OR1D2), mRNA | a start codon) |
| 55 | 56 | hOR2J2 | | NCBI | NM_030905.2 | Homo sapiens olfactory | were inserted. |
| | | | | Reference | | receptor family 2 | |
| | | | | Sequence | | subfamily J member 2 | |
| | | | | | | (OR2J2), mRNA | |
| 57 | 58 | hOR2W1 | | NCBI | NM_030903.3 | Homo sapiens olfactory | |
| | | | | Reference | | receptor family 2 | |
| | | | | Sequence | | subfamily W member 1 | |
| | | | | | | (OR2W1), mRNA | |

(ii) Class C: Metabotropic Glutamate Receptor/Metabotropic Pheromone Receptor

Class C includes, in addition to a metabotropic glutamate receptor and a metabotropic pheromone receptor, a sweet taste receptor, an umami taste receptor, a GABA (type B) receptor, and a calcium sensing receptor etc. When referred to as “metabotropic glutamate receptor/metabotropic pheromone receptor class” in the description and claims, the term refers to the whole members of Class C, unless otherwise specified. Class C receptors function as homo- or hetero-dimers and have the largest extracellular domain of about 500 to 600 residues among G protein-coupled receptors. The ligand binding domain of the extracellular domain has a structure called the Venus Flytrap module (VF™) structure.

The sweet taste receptors are receptors belonging to class C and function as T1R2/T1R3 heterodimers. It has been reported that the N-terminal domain of human T1R2 is important for acceptance of artificial sweeteners, aspartame and neotame, the transmembrane domain of human T1R3 is important for acceptance of an artificial sweetener, cyclamate, and a sweetness suppressant, lactisole, and the N-terminal domain of human T1R3 is important for acceptance of a sweetness modifier, neoculin. It has been reported that the human T1R3 C-rich domain is important for sensitivity to a sweet taste protein, brazzein.

Umami taste receptors are receptors belonging to class C and function as T1R1/T1R3 heterodimers. It has been revealed that umami substances, glutamic acid and inosinic acid (IMP), bind to the N-terminal domain of T1R1.

The amino acid sequences and the gene sequences of human T1R1, T1R2 and T1R3 constituting the sweet taste receptors and the umami taste receptors are registered in the NCBI under the accession numbers described in Table 3 below.

TABLE 3

| | | | Another | | | | Difference between |
|---|---|---|---|---|---|---|---|
| | Amino | | name | | | | a sequence used |
| Nucleotide | acid | Gene | (former | Data | ACCESSION | | in Example and |
| sequence | sequence | name | name) | source | No. | Title | a sequence in DB |
| 59 | 60 | hT1R1 | | GenBank | BK000153.1 | TPA_exp: Homo sapiens | Regarding those |
| | | | | | | taste receptor (TAS1R1) | subjected to |
| | | | | | | mRNA, complete cds | insertion of V5, |
| 61 | 62 | hT1R2 | | NCBI | NM_152232.2 | Homo sapiens taste 1 | the 3′ terminal |
| | | | | Reference | | receptor member 2 | stop codon, a |
| | | | | Sequence | | (TAS1R2), mRNA | nucleotide |
| 63 | 64 | hT1R3 | | NCBI | NM_152228.2 | Homo sapiens taste 1 | triplet, was |
| | | | | Reference | | receptor member 3 | deleted and V5 |
| | | | | Sequence | | (TAS1R3), mRNA | (including a |
| | | | | | | | stop codon) was |
| | | | | | | | inserted. |

The entire G protein-coupled receptor protein or a subunit thereof may be expressed using the construct of the present invention. For example, a construct that expresses only one subunit type, T1R2, T1R3 or T1R1, which constitutes a dimeric sweet taste receptor or umami taste receptor, is included. In that case, the other subunit constituting the dimer may be co-transfected with the use of another construct. Alternatively, a construct may be constructed such that nucleic acids encoding multiple subunits constituting a G protein-coupled receptor protein are contained in a single vector. In the present invention, a construct is constructed such that a nucleic acid encoding at least one subunit is located on the 3′ side of IRES.

(iii) Class B: Secretin Receptor Family

The class B secretin receptor family has a long N-terminal extracellular domain to form a ligand binding site. The family is classified into two subgroups: secretin-like receptors (in a narrow sense) and adhesion-type G protein-coupled receptors. The secretin-like receptors (in a narrow sense) include receptors that bind to peptide hormones such as secretin, glucagon, glucagon-like peptide (GLP), calcitonin, and parathyroid hormone. The adhesion-type G protein-coupled receptors have various domain structures at the large N-terminal site, suggesting an interaction with extracellular matrix etc.

(iv) Class F: Frizzled, Smoothened

Class F G-protein coupled receptors include 10 types of frizzled proteins that function in the Wnt signaling pathway and Smoothened proteins that function in the hedgehog signaling pathway. The class F receptors have a domain which is referred to as a cysteine-rich domain (CRD) outside the cell, comprises about 120 amino acid residues, and serves as a binding domain of a ligand such as Wnt.

In the present invention, the type of a G protein-coupled receptor or a subunit thereof is not particularly limited. The advantageous effects of the present invention have been confirmed for a plurality of types of bitter taste receptor T2R belonging to at least class A, and subunits T1R2 (constituting sweet taste receptors) and T1R3 (constituting sweet taste receptors and umami taste receptors) belonging to class C different therefrom.

In one embodiment of the present invention, preferably, the G protein-coupled receptor or a subunit thereof is, without limitation, a G protein-coupled receptor of class A rhodopsin-like receptors or a G protein-coupled receptor of class C metabotropic glutamate receptors/metabotropic pheromone receptors. In one embodiment of the present invention, the G protein-coupled receptor is a G protein-coupled receptor of the class of rhodopsin-like receptors. More preferably, the G protein-coupled receptor is a bitter taste receptor.

The biological species from which the G protein-coupled receptor protein is derived is not particularly limited. Preferably it is derived from an animal, more preferably a mammalian species. Examples of mammals include humans, rats, monkeys, dogs, pigs, sheep, horses, and mice. In one embodiment of the present invention, the G protein-coupled receptor protein is a human G protein-coupled receptor protein.

(2) G Protein α Subunit

The term “G protein (guanidine nucleotide binding protein)” is a protein belonging to a group of GTPases, and refers to a membrane receptor-related heterotrimer that is generally composed of three subunits, α, β, and γ.

G proteins bind to the inner surface of the cell membrane, wherein Gβγ dimers are tightly bound to Gα subunits. When activated upon binding of ligands to G protein-coupled receptors, G proteins bound to the G protein-coupled receptors dissociate their own GDP from the Gα subunits and bind to new GTP molecules (conversion from GDP type->GTP type). This conversion dissociates the Gα subunits, the Gβγ dimers and the G protein-coupled receptors, respectively. The thus dissociated Gα subunits activate effector proteins downstream thereof.

The G protein α subunit consists of two domains, a GTPase domain and an a helical domain. At present, at least 20 Gα subunit types are known and mainly classified into four families.

TABLE 4

| | | | | |
|---|---|---|---|---|
| G protein family | α subunit | Gene | Signaling | Examples of effects |

Gi Family

| | | | | |
|---|---|---|---|---|
| Gi/o | αi, αo | GNAO1, | Suppression of adenylate | Smooth muscle contraction, |
| | | GNAI1, | cyclase, (β/γ subunit- | decreased neuronal activity |
| | | GNAI2, | mediated) K⁺ channel | |
| | | GNAI3 | opening, Ca²⁺channel | |
| | | | closing | |
| Gt | αt (transducin) | GNAT1, | Activation of | Vision |
| | | GNAT2 | phosphodiesterase 6 | |
| Ggust | αgust (gustducin) | GNAT3 | Activation of | Gustatory sense |
| | | | phosphodiesterase 6 | |
| Gz | αz | GNAZ | Suppression of adenylate | Ion balance maintenance in |
| | | | cyclase | cochlear duct fluid of external |
| | | | | lymph and internal lymph |

Gs Family

| | | | | |
|---|---|---|---|---|
| Gs | αs | GNAS | Activation of adenylate | Increased heart rate, relaxed |
| | | | cyclase | smooth muscle, stimulation of |
| | | | | neuronal activity |
| Golf | αolf | GNAL | Activation of adenylate | Sense of smell |
| | | | cyclase | |

Gq Family

| | | | | |
|---|---|---|---|---|
| Gq | αq, α11, | GNAQ, | Activation of phospholipase | Smooth muscle contraction, |
| | α14, α15, | GNA11, | C | Ca²⁺ flow |
| | α16 | GNA14, | | |
| | | GNA15 | | |

G12/13 Family

| | | | | |
|---|---|---|---|---|
| G12/13 | α12, α13 | GNA12, | Activation of GTPase Rho | Cytoskeletal function, smooth |
| | | GNA13 | family | muscle contraction |

In the present invention, the type of G protein α subunit is not particularly limited. In one embodiment of the present invention, the G protein α subunit protein is, without limitation, a G protein α subunit protein of the Gq family, a G protein α subunit protein of the Gi family, a G protein α subunit protein of the Gs family, or a chimeric protein thereof. In one embodiment of the present invention, the G protein α subunit protein is Gα16, Gαgust or Gαolf, or a chimeric protein thereof.

The biological species from which the G protein subunit is derived is not particularly limited. Preferably the G protein subunit is derived from an animal, more preferably a mammalian species. Examples of mammals include humans, rats, monkeys, dogs, pigs, sheep, horses, and mice. In one embodiment of the present invention, the G protein is a human G protein-coupled receptor protein.

Human Gα16 is described, for example, in Proc. Natl. Acad. Sci. USA, Vol. 88, p. 5587-5591, 1991, and the mRNA sequence is registered in the GenBank under the accession number of M63904.1. The nucleotide sequence and the amino acid sequence of human Gα16 are also represented by SEQ ID NOs: 65 and 66 in the sequence listing of the present application. The gene encoding human Gα16 can also be obtained, for example, from cultured cells such as HL60 cells.

The mRNA sequence of rat Gastducin is registered in the NCBI under the accession number of NM_173139.1. The nucleotide sequence and the amino acid sequence of rat Gastducin are also represented by SEQ ID NOs: 67 and 68 in the sequence listing of the present application.

The mRNA sequence of human Gαolf is registered in the GenBank under the accession number of AF493893.1. The nucleotide sequence and the amino acid sequence of human Gαolf are also represented by SEQ ID NOS: 69 and 70 in the sequence listing of the present application.

The G protein α subunit protein may be a chimeric protein. The chimeric protein may be a fusion protein of different types of G protein α subunits derived from the same biological species, or a fusion protein of G protein α subunits of the same type derived from different biological species, or a fusion protein of G protein α subunits of different types derived from different biological species. The chimeric protein of G protein α subunit protein n may differ from the original G protein α subunit protein in characteristics such as binding specificity to a G protein-coupled receptor, binding strength or stability. With the use of a construct comprising a nucleic acid encoding such a chimeric protein, transformed cells having desired characteristics can be obtained. Further, the transformed cells having desired characteristics can be appropriately used in a method for measuring a physiological response to a substance.

In one embodiment of the present invention, the G protein α subunit protein is human Gα16/rat Gαgust. Human Gα16/rat Gαgust is a chimeric protein in which the C-terminal sequence of human Gα16 is substituted with the C-terminal sequence of rat Gαgust. It has been reported that a chimeric protein in which the C-terminal sequence of Gα16 is substituted with the C-terminal sequence of Gαgust having 37 or more amino acid residues responds to bitter taste receptor T2R (The Journal of Neuroscience, 23(19), p. 7376-7380, 2003).

The C-terminal sequence of rat Gαgust that is substituted with human Gα16 is composed of preferably at least 25 or more amino acid groups, 30 or more amino acid groups, 35 or more amino acid residues, 37 or more amino acid residues, 40 or more amino acid groups, or 43 or more amino acid residues. The C-terminal sequence of rat Gαgust that is substituted with human Gα16 is composed of preferably 60 or less amino acid residues, 50 or less amino acid groups, 45 or less amino acid groups, or 43 or less amino acid groups. In one embodiment of the present invention, the G protein α subunit protein is human Gα16/rat Gαgust43.

In the examples herein, the 3′ terminal 132 nucleotides (corresponding to a sequence encoding C terminal 43 amino acids including stop and start codons) of SEQ ID NO: 65 of human Gα16 were substituted with the corresponding sequence of rat Gastducin; that is, the 3′ terminal 132 nucleotides (corresponding to a sequence encoding C terminal 43 amino acids including stop and start codons) of SEQ ID NO: 67 (Gα16/Gαgust43 (Gα)).

(3) IRES

The construct of the present invention comprises a nucleic acid comprising an IRES sequence downstream (3′ side) of the nucleic acid encoding a G protein α subunit protein and an IRES sequence upstream of (5′ to) the nucleic acid encoding a G protein-coupled receptor protein or a subunit thereof in a single vector. IRES is an abbreviation of Internal Ribosome Entry Site (site for the entry of an internal ribosome), and is an RNA factor that enables cap-independent translation initiation in order to further increase the efficiency of protein synthesis. Although many IRESs are present in the 5′UTR (5 ‘untranslated region) of RNA viruses, it has been suggested that IRESs are also present in mammalian cells cellular mRNAs.

Viral IRESs discovered to date are roughly divided into five classes (Journal of Virology, Vol. 86, No. 3, p. 1468-1486, 2012).

The first class includes the dicistrovirus IRES group. The second class includes the IRES of the hepatitis C virus (HCV) and viruses of the genus Pestivirus, as well as the IRES of a number of structurally similar viruses.

Furthermore, three classes of IRES, referred to as type 1 to type 3, are known. These are all identified at the 5’ UTR of the family Picornaviridae.

Among these, members of type 2 are IRESs mainly found in the genera Aphthovirus, Cardiovirus, and Parechovirus. Furthermore, divergent members also exist in the genera Erbovirus and Cosavirus. Viruses of the genus Cardiovirus include Encephalomyocarditis virus (EMCV) and Theilovirus. Type 2 IRESs have a length of about 450 nucleotides, and have 3′-terminal Yn-Xm-AUG (Yn is a pyrimidine tract, n is an integer of 8-10, and m is 18-20) motif, wherein the AUG triplet may be a start codon. Type 2 IRESs contain five major domains: H, I, J, K and L. Type 2 IRESs function without the involvement of eukaryotic translation initiation factor IF4E and factors involved in ribosomal scanning, such as eIF1 and eIF1A. However, some of the type 2 IRESs require one or more IRES trans-activating factors (ITAFs). ITAF is a cellular RNA binding protein such as a pyrimidine tract binding protein (PTB).

Type 1 IRESs are those found only in the genus Enterovirus. Type 1 IRESs contain five major domains of II-VI. Type 2 IRESs have a length of about 450 nucleotides and have a 3′-terminal Yn-Xm-AUG motif like type 2. However, this motif of the type 1 IRES is separated from the start codon by a non-conserved spacer. Type 3 IRESs are those found only in hepatitis A virus.

Cellular IRES is described, for example, in GENES & DEVELOPMENT, Vol, 15, p. 1593-1612, 2001.

In the present invention, the type of IRES is not particularly limited. In one embodiment of the present invention, IRES is, but is not limited to, a viral IRES. In one embodiment of the present invention, the IRES sequence is a type 2 IRES sequence, such as an IRES sequence derived from encephalomyocarditis virus. The nucleic acid sequence of the IRES derived from encephalomyocarditis virus is registered in the GenBank under the accession number of M81861.1. The IRES sequence used in the examples herein is represented by SEQ ID NO: 71, which is partially modified based on the sequence registered in the GenBank under the accession number of M81861.1, simulating the IRES site of the pIRES2-EGFP vector (Clontech). Specifically, in SEQ ID NO: 71, G is added to nucleotide number 1 and A is added to nucleotide number 512.

(4) Polypeptide Having a Function of Enhancing Translocation of G Protein-Coupled Receptor Protein or Subunit Thereof to Cell Membrane

In one embodiment, the construct of the present invention comprises a nucleic acid encoding a polypeptide having a function of enhancing the translocation of the G protein-coupled receptor protein or a subunit thereof to the cell membrane, 5′ to the nucleic acid encoding the G protein-coupled receptor protein or a subunit thereof.

When such a construct is used, a fusion protein, in which such a polypeptide is bound to the N′-terminal side of a G protein-coupled receptor protein or a subunit thereof, is expressed. Therefore, in cells transformed with the construct, the translocation of the G protein-coupled receptor protein or a subunit thereof to the cell membrane is improved. Transformed cells, in which more G protein-coupled receptors are translocated to the cell membrane, will be more accessible by a method for measuring a physiological response to a substance.

In the present invention, the type of a polypeptide having a function of enhancing the translocation of the G protein-coupled receptor protein or a subunit thereof to the cell membrane is not particularly limited, as long as it has a function of “enhance the translocation of the G protein-coupled receptor protein or a subunit thereof to a cell membrane”. The expression “enhance the translocation of the G protein-coupled receptor protein or a subunit thereof to a cell membrane” means that when the polypeptide is bound to the N-terminal side of each G protein-coupled receptor protein or a subunit thereof, the proportion of the thus expressed G protein-coupled receptor proteins or subunits thereof translocated from the nuclei of transformed cells to a cell membrane is (significantly) increased, compared with a case where no polypeptide is bound.

Examples of such a polypeptide that enhances the translocation of the G protein-coupled receptor proteins or a subunit thereof to a cell membrane include, but are not limited to, a polypeptide comprising the whole or a portion of a rat somatostatin receptor, and a polypeptide comprising the whole or a portion of bovine rhodopsin (Archives of Physiology and Biochemistry Vol. 110 p. 137-45, 2002; Nature Genetics, Vol. 32, p. 397-401, 2002; Cell, Vol. 100, p. 703-711, 2000). In one embodiment of the present invention, a polypeptide that enhances the translocation of the G protein-coupled receptor protein or a subunit thereof to a cell membrane is a polypeptide comprising the N-terminal 45 amino acids of a rat somatostatin receptor, a polypeptide comprising N-terminal 39 amino acids of bovine rhodopsin, or a polypeptide comprising N-terminal 21 amino acids of human rhodopsin.

The mRNA sequence of the rat somatostatin receptor is registered in the NCBI under the accession number of NM_133522.1. The nucleotide sequence and the amino acid sequence of the rat somatostatin receptor are also represented by SEQ ID NOs: 72 and 73 in the sequence listing of the present application. In the examples of the description, the 5′ terminal 135 nucleotides of SEQ ID NO: 72 were linked 5′ to the nucleotide sequence encoding a G protein-coupled receptor.

The mRNA sequence of human rhodopsin is registered in the NCBI under the accession number of NM_000539.1. The nucleotide sequence and the amino acid sequence of human rhodopsin are also represented by SEQ ID NOs: 74 and 75 in the sequence listing of the present application.

(5) Vector

The construct of the present invention comprises: a nucleic acid encoding a G protein α subunit protein; a nucleic acid comprising an IRES sequence; and a nucleic acid encoding the G protein-coupled receptor protein or a subunit thereof in the stated order from the 5′ side in a single vector. The construct comprising the above nucleic acids in the order in a single vector provides effects of the increased expression level of the G protein-coupled receptor protein or a subunit thereof in transformed cells, and/or the increased responsiveness of the G protein-coupled receptor protein or a subunit thereof to a ligand substance. In the vector, it is important that the nucleic acid encoding the G protein α subunit protein, the nucleic acid comprising the IRES sequence, and the nucleic acid encoding the G protein-coupled receptor protein or a subunit thereof are arranged from 5′ in this order. It is a conventional theory that the expression level of a protein encoded by the nucleic acid on the 3′ side of IRES is not high. The present inventors conceived of the present invention based on the discovery that in the case of the G protein-coupled receptor protein or a subunit thereof, the expression level is increased by arranging the protein or a subunit thereof 3′ rather than 5′ to IRES.

Further, when the G protein-coupled receptor is a dimer such as a sweet taste receptor (T1R2/T1R3) or a umami taste receptor (T1R1/T1R3), for example, only a nucleic acid encoding one subunit constituting the dimer may be contained together with a nucleic acid encoding the G protein αsubunit protein and IRES in a vector, as described above. A nucleic acid encoding the other subunit may be contained in another vector and this vector and the afore-mentioned vector may be co-transfected.

The type of a vector is not particularly limited as long as it is an expression vector that enables the expression of a protein in desired cells. Examples thereof include, but are not limited to, plasmid vectors, phage vectors, cosmid vectors, and viral vectors. Persons skilled in the art can appropriately select a vector depending on the type of cells in which the protein is expressed.

In one embodiment of the present invention, the vector is a plasmid vector. Examples of the plasmid vector include pFRT/lacZeo, pUC12, pUC13, pUC19, pBR322, pBR325, pSH15, pSH19, pUB110, and pC194. The vectors of the pcDNA™ series (Thermo Fisher Scientific Inc., pcDNA3 and pcDNA5) can be used as expression vectors in mammalian cells. In addition to these vectors, pEAK10 (Edge biosystem) can also be used as an expression vector in mammalian cells.

The vector of the present invention preferably comprises a promoter. The type of the promoter is not particularly limited, and examples thereof include a human cytomegalovirus-derived promoter (CMV promoter) and an EF1α promoter. In one embodiment of the present invention, the vector is a plasmid vector comprising a CMV promoter or a plasmid vector comprising an EF1α promoter.

The vector of the present invention preferably comprises a selection marker for selecting transformed cells. The type of the selection marker is not particularly limited, and examples thereof include known promoters such as a drug resistance gene and a fluorescent protein gene. Containing such a selection marker makes it possible to select a cell line that stably expresses the protein.

In one embodiment of the present invention, the selection marker is a drug resistance gene. Examples of the drug resistance gene include, but are not limited to, a hygromycin resistance gene, a puromycin resistance gene, a neomycin resistance gene, and a blasticidin resistance gene.

In one embodiment of the present invention, the vector is a plasmid vector comprising a CMV promoter and a hygromycin resistance gene. An example of such a vector is the pcDNA3.1 Hygro(+) vector (Thermo Fisher Scientific Inc.) used in the examples herein.

In another embodiment of the present invention, the vector is a plasmid vector comprising an EF1α promoter and a puromycin resistance gene. An example of such a vector is pEAK10 (Edge biosystem).

The sequences of the pcDNA3.1 Hygro(+) vector and the pEAK10 vector used in the examples herein are represented by SEQ ID NOs: 80 and 81, respectively, in the sequence listing of the present application. Nucleotides 895 to 942 of SEQ ID NO: 80 correspond to a multiple cloning site (MCS) into which a target gene is inserted. Nucleotides 1892-2823 of SEQ ID NO: 81 correspond to the EF-1α promoter sequence, nucleotides 2844 to 2845 correspond to a site where the target gene is inserted, and nucleotides 3293 to 3892 correspond to the puromycin resistance gene sequence.

II. Transformed Cells

The present invention also relates to transformed cells into which the construct of the present invention is introduced. The type of host cells for preparation of transformed cells by gene transfer of the construct of the present invention is not particularly limited as long as the cells can be transformed by introducing an exogenous nucleic acid via gene transfer.

For example, the cells are selected from the group consisting of animal cells, insect cells, plant cells, yeasts and bacteria. The origin of animal cells to be used herein is not particularly limited. Preferably, such cells refer to cells derived from an animal belonging to the phylum Chordata. The phylum Chordata includes Agnatha and Gnathostomata, and the Gnathostomata includes Mammalia, Ayes, Amphibia, Reptilia and the like. Preferably, the animal cells are generally cells derived from an animal belonging to the Mammalia referred to as mammals. Examples of mammals include, but are not limited to, preferably mice, rats, humans, monkeys, pigs, dogs, sheep, and goats.

Cells derived from mammals include “established cell lines”, which are cultured cells maintained outside the body for a long period of time to have certain stable properties, and thus the semi-permanent passage thereof can be performed. For example, there are established cell lines derived from various tissues of various biological species including humans, such as PC12 cells (derived from rat adrenal medulla), CHO cells (derived from Chinese hamster ovary), HEK293 cells (derived from human fetal kidney), HL-60 cells (derived from human white blood cells), HeLa cells (derived from human cervical cancer), Vero cells (derived from African green monkey kidney epithelial cells), MDCK cells (derived from canine renal tubular epithelial cells) and HepG2 cells (derived from human liver cancer).

The origin of plant cells herein is not particularly limited. The cells of plants including bryophytes, pteridophytes, and spermatophytes are target plant cells. Plants from which cells of spermatophytes are derived include both monocotyledonous and dicotyledonous plants. The origin of insect cells herein is also not particularly limited. The types of archaebacteria and bacteria to be used herein are also not particularly limited.

Examples of host cells for preparation of the transformed cells of the present invention, which can be used herein, include, but are not limited to, eukaryotic cells such as HEK 293 cells, CHO cells, 32D cells, HeLa cells, COS cells, and BHK cells. In one embodiment of the present invention, the host cells are HEK 293 cells.

Methods for the gene transfer (transformation) into host cells using the construct of the present invention are not particularly limited. Methods suitable for gene transfer of nucleic acids into animal cells, plant cells, and bacteria, are known for each of these cells (e.g., MOLECULAR CLONING: A Laboratory Manual (Fourth Edition), Michael R Green and Joseph Sambrook, 2012, (Cold Spring Harbor Laboratory Press)). Persons skilled in the art can employ appropriate gene transfer methods depending on the type of a vector, the type of cells, and the like. For example, in the case of non-viral vectors such as plasmid vectors, gene transfer methods such as a lipofection method, an electroporation method and a microinjection method are known.

The transformed cells may be cells (stable expression cell line) stably expressing a G protein-coupled receptor protein or a subunit thereof or cells transiently expressing the same (transiently expressing cells). The “stable expression cell line” is, for example, a cell line stably expressing the G protein-coupled receptor protein or a subunit thereof, as confirmed using a selection marker that a nucleic acid contained in the construct has been stably incorporated by gene transfer into the genome of host cells. The “transiently expressing cells” are not confirmed to undergo stable incorporation of the nucleic acid contained in the construct into the genome of the host cells, and thus are not confirmed to stably express the G protein-coupled receptor protein or a subunit thereof.

The transformed cells of the present invention have the increased expression level of a G protein-coupled receptor protein or a subunit thereof, and/or the increased responsiveness of the G protein-coupled receptor protein or a subunit thereof to a ligand substance. The expression “the increased expression level of a G protein-coupled receptor protein or a subunit thereof” means that the expression level of the G protein-coupled receptor protein or a subunit thereof is increased compared to a case where the same is expressed using conventional techniques. The expression “the increased responsiveness of a G protein-coupled receptor protein or a subunit thereof to a ligand substance” means that the responsiveness of the G protein-coupled receptor protein or a subunit thereof to a ligand substance is increased compared to a case where the same is expressed using conventional techniques prior to the present invention.

The “conventional techniques” includes, for example, the following embodiments.

(1) Double-gene transfer
(1a) Host cells are co-transfected with a vector comprising a nucleic acid encoding a Gα protein and a vector comprising a nucleic acid encoding the G protein-coupled receptor protein or a subunit thereof;
(1b) A cell line in which a Gα protein is stably expressed is transformed with a vector comprising a nucleic acid encoding the G protein-coupled receptor protein or a subunit thereof.
(2) Co-expression vector comprising IRES

Transformation is performed using a single vector comprising, from 5′ in this order: a promoter; a nucleic acid encoding the G protein-coupled receptor protein or a subunit thereof; IRES; and a nucleic acid encoding a Gα protein.

The transformed cells of the present invention are superior in stability to a case where a G protein-coupled receptor protein or a subunit thereof is expressed using conventional techniques. Examples of the stability include stability under normal culture conditions for cells and stability under storage conditions such as conditions for cryopreservation. For example, in the examples herein, even in the case of about 1 month of cryopreservation, the response intensity to a ligand was hardly decreased and was 0.8 or more as compared to that before cryopreservation. On the other hand, in the case of double-gene transfer (double stable), the response intensity to a ligand was decreased to less than 0.6 after freeze-drying.

III. Method for Measuring Physiological Response to Substance

The present invention further relates to a method for measuring a physiological response to a substance. The measuring method of the present invention comprises a step of adding a substance to the transformed cells of the present invention and measuring the physiological response to the substance.

The “substance” is not particularly limited as long as it is a substance that can bind to a G protein-coupled receptor and function as an agonist. Such substance causes a structural change in a G protein-coupled receptor, and activates a G protein bound to the inside of the membrane to perform signaling, and examples thereof include chemical substances contained in foods and beverages, odor molecules, neurotransmitters, hormones, etc. Signals induce physiological responses in intracellular signal networks such as olfactory sense, gustatory sense, vision, neurotransmission, metabolism, cell differentiation and cell proliferation, and inflammatory responses and immune responses. Substances differ depending on the types of G protein-coupled receptors.

In one embodiment, the substance is a bitter substance, an odorant, an umami substance or a sweet substance, or a modulator to these substances.

The “bitter substance” is not particularly limited as long as it is a substance that a human can sense its bitter taste. Examples thereof include denatonium benzoate, strychnine, β-glucopyranoside (salicin), aristolochic acid, a bitter substance having an isothiocyanate group (e.g., phenylthiocarbamide (PTC), propylthiouracil (PROP)), diphenydol, quinine, absinthin and denatonium.

The “odorant” is a low molecular weight compound having a molecular weight of about 30 to 300. For example, the basic skeleton of serotonin, which is a brain hormone, is a substance with a fecal odor called indole, and the skeleton of dopamine or adrenalin resembles vanillin, which is the odor of vanilla. Any substance can be an odorant, as long as it is a low molecular weight organic compound having the property of volatility. Odors are emitted from various foods, plants and animals, and even humans said to have deteriorated sense of smell can identify about 10,000 odor types. Examples of “odorant”, and specific examples of “odorant” include muscone which is a main component of musk (mush) odor, civetone which is a main component of civet, and α-ionone which exudes a flower scent of violet, α-terpineol which is a main component of a flower scent of lilac, (−) 5R-carvone which produces a scent of spearmint, and further, methylamine, dimethylamine, trimethylamine, etc., which produce fish odor.

The “umami substance” is not particularly limited as long as it is a substance, the umami taste of which can be detected by humans, for example. Examples thereof include glutamic acid and inosinic acid (IMP).

The “sweet substance” is not particularly limited as long as it is a substance the sweetness of which can be sensed by humans. Examples thereof broadly include saccharide sweeteners such as glucose, fructose, galactose, raffinose, xylose, sucrose, maltose, lactose, starch syrup, isomerized sugar, isomaltooligosaccharide, fructooligosaccharide, galactooligosaccharide, xylooligosaccharide, lactosucrose, soy oligosaccharide, trehalose, sorbitol, mannitol, maltitol, xylitol, erythritol, lactitol, isomaltol, reduced sugar syrup, reduced palatinose, Wasanbon (Japanese cured sugar), black sugar, soft brown sugar, honey, molasses, licorice extract, and maple syrup, and non-saccharide sweeteners such as aspartame, saccharin, dulcin, stevioside, stevia extract, glycyrrhizin, acesulfame-K, sucralose (registered trademark; San-Ei Gen F. F. I., Inc.), cyclamate, alitame, neotame, perillartin, monellin, and curculin (registered trademark; ADEKA).

The term “a bitter substance, an odorant, an umami substance or a modulator of a sweet substance” refers to a substance that does not independently produce bitterness, smell, umami taste or sweetness, but enhances, modifies or suppresses the gustatory sense and the olfactory sense of each substance when accepted together with a bitter substance, an odorant, an umami substance or a sweetener.

Examples of “a modulator of a bitter substance” include Benecort BMI (Kao) and probenecid. Examples of “a modulator of an odorant” include olfactory inhibitors such as 2,4,6-trichloroanisole and geraniol. Examples of “a modulator of a sweetener” include a sweetness suppressant, lactisole, a sweetness suppressant, gymnemic acid, and a sweetness modifier, neoculin.

A physiological response to a substance is measured using the transformed cells of the present invention as follows, for example.

A predetermined number of the transformed cells of the present invention are seeded in each well of a microplate having a large number of wells (24 wells, 48 wells, 96 wells, 384 wells, etc.) (e.g., 10,000 to 500,000 cells/well) and then the cells are cultured in a predetermined medium (e.g., DMEM medium). After cell culture, when a specific substance is added, a physiological response generated in a stable culture cell line is measured, and the physiological response to the substance is evaluated based on the measurement result. In measuring physiological responses, events that change with activation of G protein-coupled receptors are appropriately selected depending on the type of a substance.

For example, when a gustatory sense receptor such as a bitter taste receptor, a sweet taste receptor, and an umami taste receptor is activated, intracellular calcium concentration is increased through the signal transduction process mediated by intracellular second messengers (IP3 (inositol triphosphate), DAG), etc. Therefore, physiological responses to be measured include changes in intracellular second messengers, changes in intracellular calcium concentration, and the like, which take place in association with activation of taste receptors. Thus, it is possible to measure a physiological response by measuring changes in intracellular second messengers or calcium concentration.

For example, such measurement can be performed by a calcium imaging method that involves observing a change in intracellular calcium concentration caused by stimulation of gustatory senses such as bitter taste, sweet taste or umami taste using a fluorescent calcium indicator. The fluorescent calcium indicator requires a change in fluorescence characteristics at a calcium concentration that can be physiologically changed, and induction of such a change in a calcium-specific manner. Examples of the fluorescent calcium indicator include Fluo-8 (registered trademark) (AAT Bioquest, Inc.) (excitation wavelength of 490 nm, fluorescence wavelength of 514 nm), Fluo-4 AM (AAT Bioquest, Inc.) (excitation wavelength of 490 nm, fluorescence wavelength of 525 nm), and Fura 2 AM (Thermo Fisher, Inc.) (excitation wavelength of 340 nm, fluorescence wavelength of 380 nm).

For measuring a physiological response to an odorant, for example, a technique for measuring a change in intracellular cAMP concentration induced by the stimulation of olfactory sense by an odorant is employed for measuring a physiological response to the odorant. Examples of a method for measuring intracellular cAMP include techniques that involve measuring by a fluorescent polarization immunoassay or a competitive immunoassay using cAMP labeled with a fluorescence indicator or an enzyme such as HRP and an anti-cAMP antibody, and techniques that involve performing gene transfer of a vector containing luciferase inserted therein to a site downstream of a cAMP response element (CRE) and then measuring by luciferase assay.

The present invention comprises the use of the transformed cells according to the present invention for measuring a physiological response to a substance.

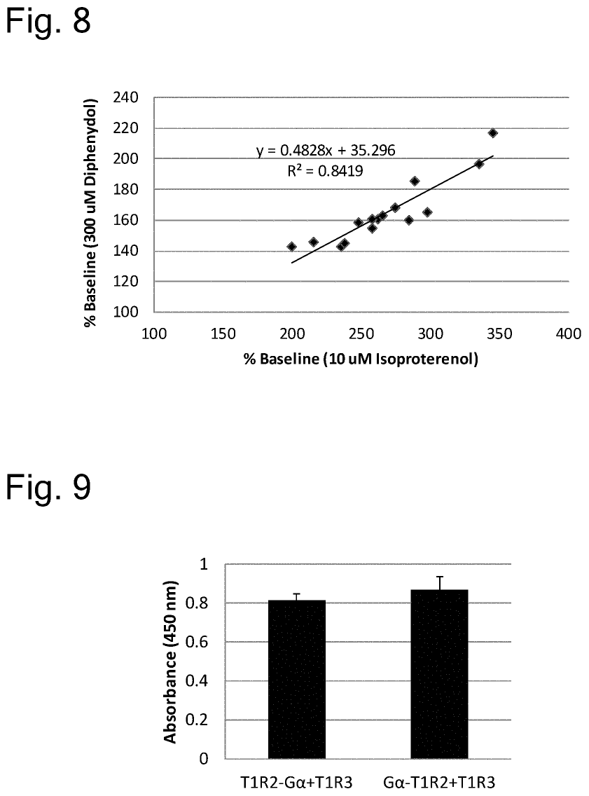

The present invention comprises, in a single vector, a nucleic acid encoding a G protein α subunit protein and a nucleic acid encoding a G protein-coupled receptor protein or a subunit thereof. Therefore, it is presumed that the expression levels of the G protein α subunit protein and the G protein-coupled receptor protein in the transformed cells are positively correlated. In the transformed cells of the present invention, the responsiveness of the G protein-coupled receptor to a G protein-coupled receptor agonist and the response intensity to a Gα agonist exhibit a high positive correlation (Example 7, FIG. 8). Therefore, it is also possible to estimate the physiological response intensity to a substance by measuring the response intensity of a G protein α subunit to a Gα agonist in the transformed cells to which the substance (G protein-coupled receptor agonist) has been added.

IV. Kit for Use in a Method for Measuring Physiological Response to Substance

The present invention further relates to a kit comprising the transformed cells of the present invention, which is used for a method for measuring a physiological response to a substance by adding a substance to the transformed cells, and then measuring a physiological response induced by the substance.

The kit of the present invention may comprise, in addition to the transformed cells of the present invention, materials necessary for use in a method for measuring a physiological response to a substance, instructions for use, and the like. Examples of materials necessary for use in the method for measuring a physiological response to a substance include, a medium for maintaining and culturing transformed cells, and reagents (e.g., a fluorescent calcium indicator for measuring calcium concentration etc.) for measuring second messengers or the concentration of calcium resulting from a physiological response to a substance.

The present invention relates to an apparatus comprising transformed cells, which is used for a method for measuring a physiological response to a substance by adding a substance to the transformed cells and measuring a physiological response induced by the substance. The apparatus of the present invention includes the kit of the present invention, and equipment for measuring second messengers or the concentration of calcium resulting from a physiological response to a substance.

As an example of the apparatus for measuring a physiological response to a taste substance such as a bitter substance, an odorant, an umami substance or a sweet substance, or modulators of these substances, an “artificial lipid membrane taste sensor” has been developed by Intelligent Sensor Technology Inc., (http://www.insent.co.jp/products/taste_sensor_index.html), for example. This taste sensor is a device for detecting a displacement in the membrane potential of the artificial lipid membrane due to electrostatic interaction or hydrophobic interaction with various taste substances in the form of sensor outputs by a computer. For example, a taste sensing system TS-5000Z is marketed by the Intelligent Sensor Technology Inc. The apparatus of the present invention utilizes the transformed cells of the present invention instead of the artificial lipid membrane in such a sensor. Specifically, the transformed cells of the present invention are immobilized in the apparatus instead of an artificial membrane, a substance is allowed to act, a change in intracellular second messenger or calcium concentration is measured, and thus a physiological response to the substance is measured. The apparatus of the present invention can be used repeatedly for measurement by removing test substances by washing or the like after use.

### EXAMPLES

Hereinafter, the present invention will be described in detail based on examples, but the present invention is not limited to these examples. Persons skilled in the art can easily make modifications and changes to the present invention based on the description, and these are included in the technical scope of the present invention.

### Example 1 Construction of Construct and Method for Preparing Construct

In this example, a construct for expressing a G protein-coupled receptor protein was prepared. The construct of the present invention comprises: a nucleic acid encoding a G protein α subunit protein; a nucleic acid comprising an IRES sequence; and a nucleic acid encoding the G protein-coupled receptor protein in the stated order from the 5′ side in a single vector.

The following three gene types were amplified by PCR respectively.

A human bitter taste receptor (T2R16) expression gene (ssr/T2R16N5) prepared by the addition of a gene sequence ranging from the start codon to the 135th nucleotide of rat somatostatin receptor 3 (ssr3) to the 5′ end and a V5 epitope sequence to the 3′ end.

Note that the V5 epitope sequence is an antibody recognition tag for examining the protein expression level of T2R (nucleotide sequence: SEQ ID NO: 76).
Encephalomyocarditis virus-derived IRES, and
Gα16/gust expression gene (Gα16/Gαgust43) (SEQ ID NO: 77) prepared by substitution of terminal 129 nucleotides of human G protein αsubunit G16 with terminal 129 nucleotides of rat gustducin (Ggust).

The thus obtained gene fragment was inserted into the multiple cloning site of pcDNA3.1 Hygro(+) vector (Thermo Fisher Scientific Inc.) using an In-Fusion HD Cloning Kit (Clontech). The insertion order is as follows.

Vector of the present invention: Gα-T2R16

Gα16/Gαgust43-IRES-ssr/T2R16N5

Vector of a comparative example: T2R16-Gα
ssr/T2R16N5-IRES-Gα16/Gαgust43

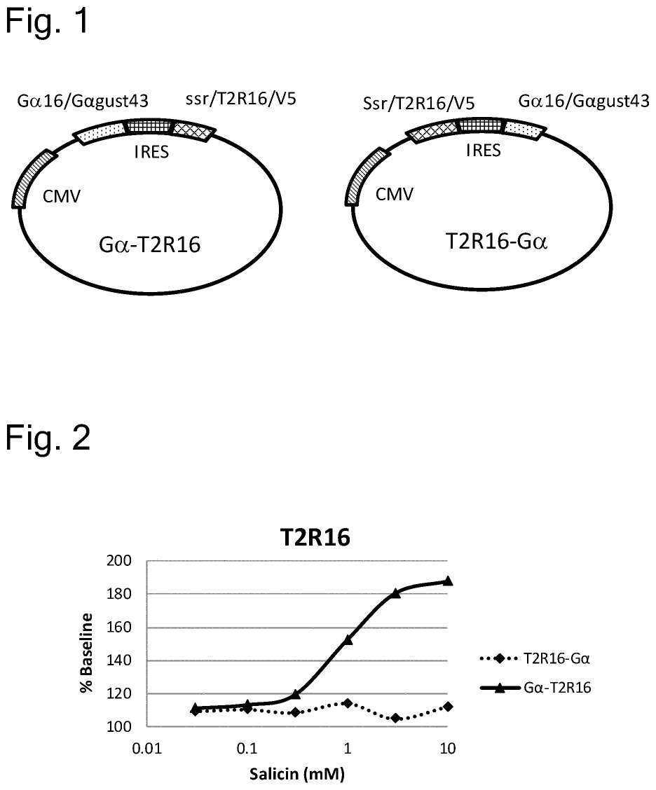

The construction of each vector is depicted in FIG. 1.

### Example 2 Preparation of Stable Expression Cell Line Containing the Construct of the Present Invention

In this example, a cell line (T2R16 stable expression cell line) stably expressing the bitter taste receptor T2R16 was prepared by incorporating the construct (Gα-T2R16) of the present invention.

Specifically, into 1×10⁶ cells of the human embryonic kidney cell line HEK293T (obtained from RIKEN) seeded in a culture vessel, 5 μg of each of the Gα-T2R16 and T2R16-Gα plasmid vectors prepared in Example 1 was introduced through the use of lipofectamine (registered trademark) 3000 (Thermo Fisher Scientific Inc.). After 48 hours of culture, drug resistance screening was performed by adding 100 μg/mL hygromycin B to the medium and then culturing for about 15 days. Cells that had acquired drug resistance were cloned by seeding these cells in each well of a 96-well plate at one cell per well. After about 10 days of culture, a stable expression cell line was obtained.

### Example 3 Measurement of Response of Stable Expression Cell Line to Bitter Substance

### (1) Measurement of Response of Gα-T2R16 Stable Expression Cell Line

In this example, the response of the T2R16 stable expression cell line of Example 2 to a bitter substance was measured.

The T2R16 stable expression cell line prepared based on the method described in Example 2 was seeded at 2×10⁴ cells/well in a 96-well plate. The culture media for cells contained a green fluorescent calcium indicator Fluo-8 (registered trademark) (AAT Bioquest, Inc.), and the uptake of Fluo-8 into the cell lines was examined. Fluo-8 (registered trademark) is suitable for detection of intracellular calcium dynamics and is an indicator of GPCR activity.

Cells incorporating Fluo-8 (registered trademark) were excited at 490 nm and the fluorescence intensity at 514 nm was measured. During measurement, T2R16 agonist, salicin (10, 3, 1, 0.3 or 0.1 mM), was added, and the maximum fluorescence intensity during 40 seconds after addition was obtained. The increase rate of the maximum fluorescence intensity relative to the fluorescence intensity at the start of the measurement was calculated as the response intensity of T2R16 to the agonist. The results are depicted in FIG. 2.

As depicted in FIG. 2, only the cell line containing the Gα-T2R16 plasmid exhibited responses to salicin. In the cell line containing the T2R16-Gα plasmid, almost no response to salicin was observed, and a clone exhibiting a response could not be isolated.

(2) Measurement of Response of Gα-T2R40 and Gα-T2R43 Stable Expression Cell Lines

Responses of cell lines stably expressing bitter taste receptor subtypes, T2R40 and T2R43, other than T2R16, were also measured by the same method as in Example 3(1).

Specifically, by the same method as in Example 1 and Example 2, a Gα-T2R40 stable expression cell line (containing Gα16/Gαgust43-IRES-ssr/T2R40N5) and a Gα-T2R43 stable expression cell line (containing Gα16/Gαgust43-IRES-ssr/T2R43/V5) were prepared.

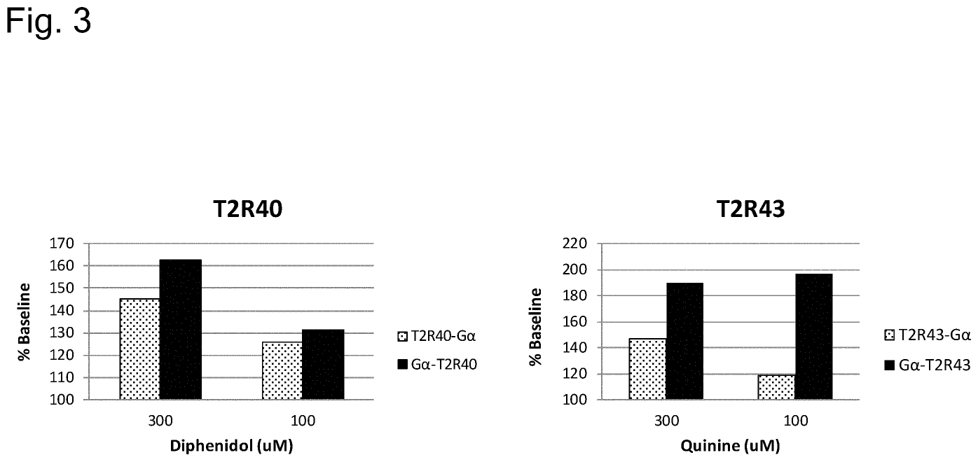

The Gα-T2R40 stable expression cell line and the Gα-T2R43 stable expression cell line were prepared by the same method as in Example 2. Each agonist (T2R40: diphenydol, T2R43: quinine) was allowed to act on these cell lines at 100 μM or 300 μM, and then the maximum fluorescence intensity during 40 seconds after addition was obtained. The increase rate of the maximum fluorescence intensity relative to the fluorescence intensity at the start of measurement was calculated as the response intensity of T2R40 and T2R43 to the agonists. The results are depicted in FIG. 3. As compared with T2R40-Gα and T2R43-Gα, Gα-T2R40 and Gα-T2R43 exhibited higher response intensity to each agonist.

### Example 4 Measurement of Response of Transiently Expressing Cells to Bitter Substance

Into HEK293T seeded at 2×10⁴ in a 96-well plate, 0.1 μg of each of the Gα-T2R16 and the T2R16-Gα plasmid vectors was introduced using lipofectamine (registered trademark) 3000. After 30 hours of culture, response intensity to salicin was measured using the same technique as in Example 3 (1) (maximum fluorescence intensity during 40 seconds after the addition of salicin). Note that drug resistance screening with hygromycin B as in Example 2 was not performed (transiently expressing cells).

Furthermore, as a comparative example, the following cells were also prepared other than T2R16-Gα expressing cells, and then the response to salicin was measured:

cells (cotransfection), prepared by simultaneous gene transfer of a total of 0.1 μg of a Gα16/Gαgust43gust expression vector and a ssr-T2R16-V5 expression vector comprising a Gα16/Gαgust43 expression gene and a ssr-T2R16-V5 expression gene, respectively, via incorporation thereof into pcDNA3.1 Hygro(+) vectors for transient expression of the genes; and

cells (Gα stable), prepared by the gene transfer of 0.1 μg of a ssr-T2R16-V5 expression vector into a Gα16/Gαgust43 stable expression cell line (subjected in advance to drug resistance screening using hygromycin B) prepared by the same technique as in Example 2 for transient expression of the genes.

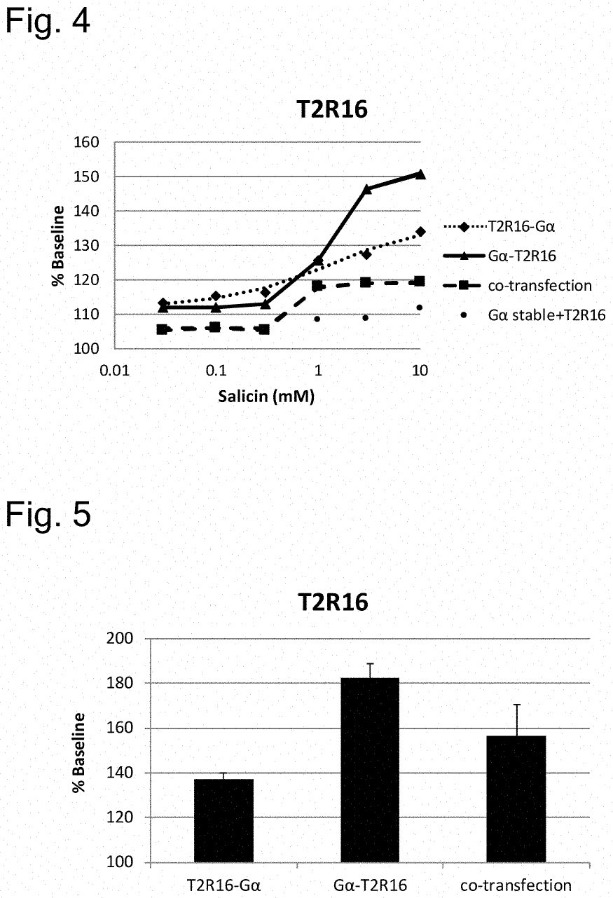

The results are depicted in FIG. 4. As can be seen from the results in FIG. 4, it was confirmed that Gα-T2R16 exhibited response intensity higher than that of T2R16-Gα even in the transient expression system, and also higher than that of the previously reported transient expression systems (co-transfection and Gα stable).

### Example 5 Expression Levels of Gα16/GαGust43 and Bitter Taste Receptor T2R16

In general, IRES-mediated translation of mRNA is considered to be less efficient compared to cap-dependent translation from the 5′ end. In this example, the expression levels of Gα16/Gαgust43 and bitter taste receptor T2R16 were examined for cap-dependent translation and IRES-dependent translation.

(1) Gα16/Gαgust43 Expression Level

Transiently expressing cells, “Gα-T2R16 (the present invention)”, transiently expressing cells, “T2R16-Gα”, and transiently expressing cells, “co-transfection” prepared in Example 4, were subjected to measurement and comparison of response intensity to a T2R independent Gα16 agonist, isoproterenol (3 μM) by the same method as in Example 3. The results are depicted in FIG. 5.

The response intensity to isoproterenol is considered to be proportional to the expression level of Gα16. As depicted in FIG. 5, it was confirmed that the expression level of T2R16-Gα resulting from IRES-mediated translation of Gα16/Gαgust43 is lower than that of “Gα-T2R16” and “co-transfection” resulting from cap-dependent translation of Gα16/Gαgust43. Specifically, it was demonstrated that the expression level of Gα16/Gαgust43 is cap-dependent>IRES-dependent.

(1) T2R16 Expression Level

T2R16-Gα, Gα-T2R16 (the present invention) and ssr-T2R16-V5 alone (T2R16 single)-transiently expressing cells were compared for the expression level of T2R16 by an In cell ELISA method. For the In cell ELISA, an In Cell ELISA Kit (BOO Bioscientific) was used, and for detection of T2R16, an anti-V5-HRP antibody (Thermo Fisher Scientific Inc.) for recognizing V5 epitope added to the C-terminus was used. The relative expression level of T2R16 was obtained by measuring the absorbance at 450 nm to determine the color development of the HRP substrate.

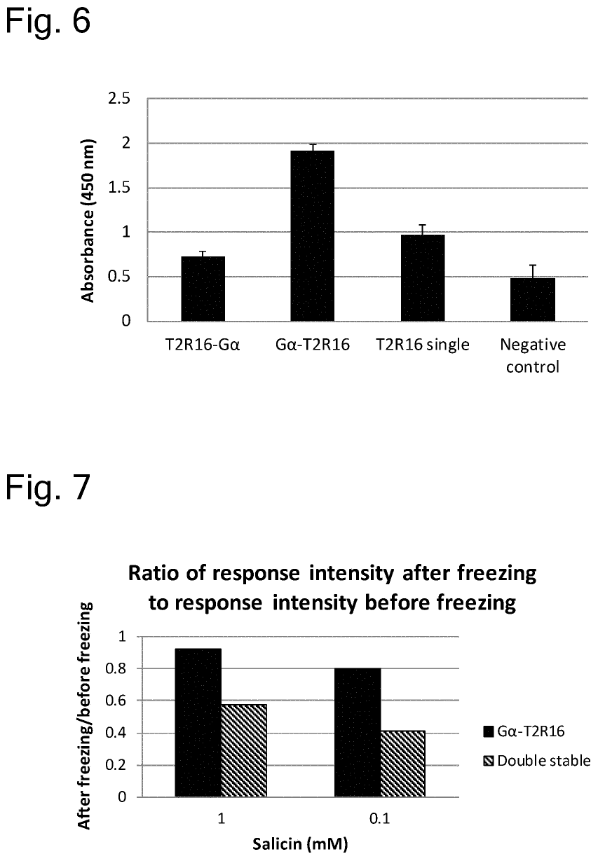

The results are depicted in FIG. 6. As a result, it was confirmed that the expression level of T2R16 of Gα-T2R16 resulting from IRES-mediated translation of “T2R16” was higher than that of “T2R16-Gα and T2R16 single” resulting from cap-dependent translation of T2R16. Specifically, it was demonstrated that the expression level of T2R16 was cap-dependent<IRES-dependent. Similar results were also obtained upon the use of Gα16 having a property of binding with T2R lower than that of Gα16/Gαgust43 (data not shown).

Therefore, it was demonstrated that the Gα-T2R16 of the present invention causes the high levels of expression of both Gα 16/Gαgust43 (and Gal 6) and T2R16. Furthermore, similar results were observed for subtypes (T2R40 and T2R43) other than the bitter taste receptor T2R16 (data not shown).

### Example 6 Storage Stability of Gα-T2R16 Stable Expression Cell Line

In this example, response intensity before and that after cryopreservation of the Gα-T2R16 stable expression cell line were examined.

Gα-T2R16 in this example was prepared in the same manner as in the case of the Gα-T2R16 stable expression cell line in Example 2, but pEAK10 (Edge biosystem, Inc.) was used as an expression vector and “ssr/T2R16” (without V5) was inserted instead of “ssr/T2R16/V5”. Further, drug resistance screening was performed with 1 μg/mL puromycin instead of hygromycin B.

As a comparative example, into the Gα16/Gαgust43 stable expression cell line (subjected in advance to drug resistance screening with puromycin) prepared using the same technique as in Example 2, 5 μg of ssr-T2R16 expression vector was introduced and then drug resistance screening was performed using puromycin. The thus prepared stable expression cell line (Double stable) stably expressing Gα16/Gαgust43+T2R16 was used.

Before and after cryopreservation of the cell line, the response intensity to salicin (1 or 0.1 mM) was examined in the same manner as in Example 3. For cryopreservation, CELLBANKER (Nippon Zenyaku Kogyo Co., Ltd.) was used, and cells were stored under liquid nitrogen for 1 month. The temperature for the cryopreserved cells was brought back to normal temperature, cells were passaged at least twice, and then the response intensity after cryopreservation was measured. The results are depicted in FIG. 7.

As a result, almost no effect due to freezing was confirmed among the Gα-T2R16 stable expression cell line. On the other hand, in the case of Double stable, cell lines exhibiting a significant decrease in response intensity after freezing were occasionally observed. An example of the results is depicted in FIG. 7. In FIG. 7, the Gα-T2R16 stable expression cell line exhibited the ratio of response intensity after freezing/response intensity before freezing of 0.8 or more in cases of both salicin concentrations of 1 mM and 0.1 mM. On the contrary, in “Double stable”, the ratio was less than 0.6 (in particular, 0.4 or less in the case of 0.1 mM salicin).

### Example 7 Response Intensity of Gα-T2R40 Stable Expression Cell Line to T2R40 Agonist and Response Intensity of the Same to Gα Agonist

In this example, the Gα-T2R40 stable expression cell line of the present invention was examined for the correlation between the response intensity to a T2R40 agonist (diphenydol) and the response intensity to a Gα agonist (isoproterenol).

As described in Example 3 (2), a Gα-T2R40 stable expression cell line was prepared by the same method as in Example 1 and Example 2, and 15 clones were prepared by cloning. The correlation between responses to isoproterenol (10 μM) and responses to diphenydol (300 μM) in these 15 clones is depicted in FIG. 8. As a result, a high correlation was obtained between the responses to the T2R40 agonist, diphenydol, and the responses to the Gα16 agonist, isoproterenol.

Therefore, through the method using the construct of the present invention, a desired cell line that expresses both Gα subunit and T2R proteins can be obtained by only one evaluation of either T2R or Gα subunit protein. On the other hand, when a conventional method is employed, a gene for expressing the Gα subunit protein and a gene for expressing the T2R protein are successively introduced into cells, and after every introduction, screening should be performed while evaluating whether or not each protein is stably expressed. Specifically, after a Gα stable expression cell line is prepared and then screened for (evaluation 1), a gene encoding the T2R protein is introduced and thus a cell line expressing the T2R protein at a high level (evaluation 3) while maintaining the high-level expression of the Gα protein (evaluation 2) is obtained. Hence, evaluation should be performed 3 times to obtain a desired cell line.

According to the present invention, the working efficiency for obtaining a Gα-T2R40 stable expression cell line is remarkably improved.

### Example 8 IRES-Mediated Expression System 1 for Sweet Taste Receptor T1R2/T1R3: Examination of T1R2

A sweet taste receptor is a G protein-coupled receptor of the metabotropic glutamate receptor/metabotropic pheromone receptor class consisting of a combination of two subunit types of T1R2 and T1R3. In this example, T1R2 of sweet taste receptor T1R2/T1R3 in the IRES-mediated expression system was examined.

(1) Preparation of Expression Vector

Human sweet taste receptor subunit T1R2 and human sweet taste receptor subunit T1R3 and IRES and Gα16/Gαgust43 (Gα) described in Example 1 were prepared. They were inserted into the multiple cloning site of pcDNA3.1 Hygro (+) vectors (Thermo Fisher Scientific Inc.) as follows, thereby obtaining each construct.

### Construct Gα-T1R2

Gα16/Gαgust43-IRES-T1R2N5

Construct T1R2-Gα

ssr/T1R2N5-IRES-Gα16/Gαgust43

### Construct T1R3

T1R3

(2) T1R2 Expression Level

Through double-gene transfer of Gα-T1R2 and T1R3 by the same method as in Example 4, a cell line (Gα-T1R2+T1R3) transiently expressing Gα-T1R2 and T1R3 was obtained. As a comparative example, a cell line (T1R2-Gα+T1R3) transiently expressing T1R2-Gα and T1R3 was obtained by double-gene transfer thereof.

The expression level of T1R2 was examined by the In cell ELISA using an In Cell ELISA Kit (BOO Bioscientific) by the same method as in Example 5 (2). The results are depicted in FIG. 9. The expression level of T1R2 tended to be higher in “Gα-T1R2+T1R3” than that in “T1R2-Gα+T1R3”. As in the bitter taste receptor (T2R), the expression level was cap-dependent<IRES-dependent.

(3) Gα16/GαGust43 Expression Level

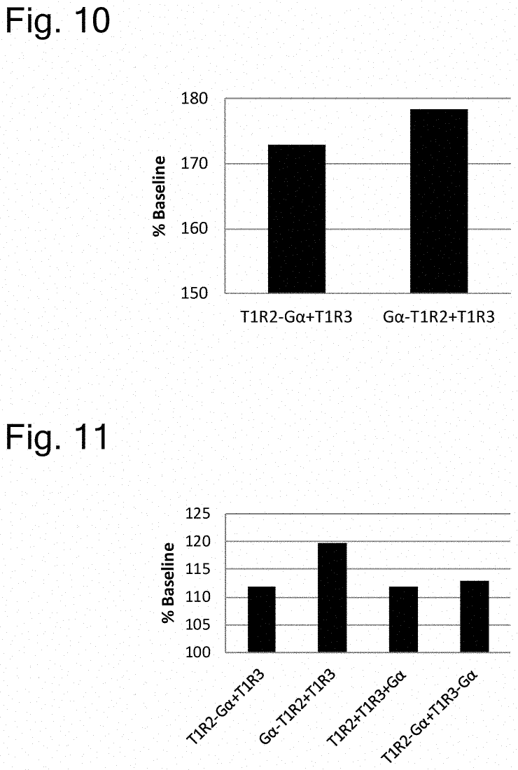

The expression level of Gα16/Gαgust43 was examined for “Gα-T1R2+T1R3” and “T1R2-Gα+T1R3” by the same method as in Example 5(1). 10 μM of isoproterenol, which is a Gα16 agonist, was used. As depicted in FIG. 10, the expression level of Gα16/Gαgust43 was higher in “Gα-T1R2+T1R3” than the other. As in the bitter taste receptor (T2R), the expression level was cap-dependent>IRES-dependent.

(4) Response to T1R2 Agonist

“Gα-T1R2+T1R3” and “T1R2-Gα+T1R3” were examined for the response to a T1R2 agonist. Aspartame (10 mM) was added and the maximum fluorescence intensity during 40 seconds after addition was obtained. The increase rate of the maximum fluorescence intensity relative to the fluorescence intensity at the start of measurement was calculated as the response intensity of T1R2 to the agonist.

As a comparative example, in addition to “T1R2-Gα+T1R3”, “cells (T1R2+T1R3+Gα) co-transfected with three types of vectors each comprising a gene encoding each protein of T1R2, T1R3 and Gα16/Gα gust43”, and “cells (T1R2-Gα+T1R3-Gα) co-transfected with T1R2-Gα and T1R3-Gα” were used. As depicted in FIG. 11, “Gα-T1R2+T1R3” exhibited the highest response to the T1R2 agonist.

### Example 9 IRES-Mediated Expression System 2 for Sweet Taste Receptor T1R2/T1R3: Examination of T1R3

In this example, T1R3 of sweet taste receptor T1R2/T1R3 in the IRES-mediated expression system was examined.

(1) Preparation of Expression Vector

Human sweet taste receptor subunit T1R2 and human sweet taste receptor subunit T1R3 and IRES and Gα16/Gαgust43 (Gα) described in Example 1 were prepared. They were inserted into the multiple cloning site of pcDNA3.1 Hygro (+) vectors (Thermo Fisher Scientific Inc.) as follows, thereby obtaining each construct.

### Construct Gα-T1R3

Gα16/Gαgust43-IRES-ssr/T1R3/V5

Construct T1R3-Gα

ssr/T1R3N5-IRES-Gα16/Gαgust43

### Construct T1R2

T1R2

(2) T1R3 expression level

Through double-gene transfer of Gα-T1R3 and T1R2 by the same method as in Example 4, a cell line (Gα-T1R3+T1R2) transiently expressing Gα-T1R3 and T1R2 was obtained. As a comparative example, a cell line (T1R3-Gα+T1R2) transiently expressing T1R3-Gα and T1R2 was obtained by double-gene transfer thereof.

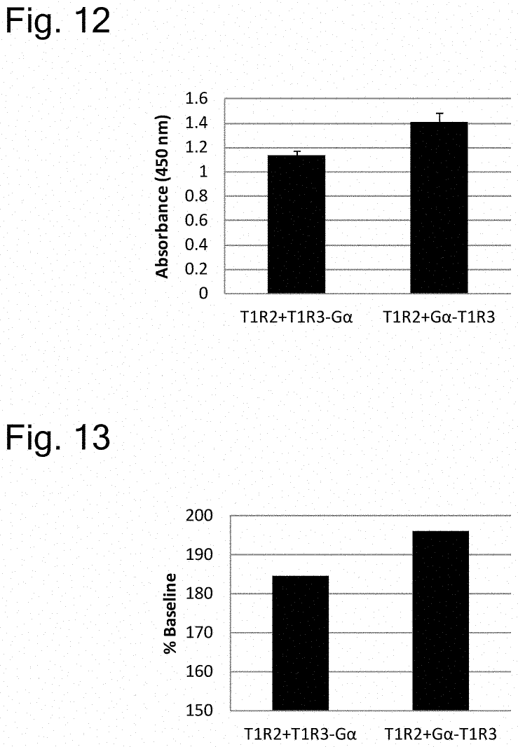

The expression level of T1R3 was examined by the In cell ELISA using an In Cell ELISA Kit (BOO Bioscientific) by the same method as in Example 5(2). The results are depicted in FIG. 12. The expression level of T1R3 was higher in “Gα-T1R3+T1R2” than in “T1R3-Gα+T1R2”. The expression level of T1R3 was cap-dependent<IRES-dependent.

(3) Gα16/Gαgust43 Expression Level

“Gα-T1R3+T1R2” and “T1R3-Gα+T1R2” were examined for the expression level of Gα16/Gαgust43 by the same method as in Example 5(1). 10 μM of isoproterenol, which is a Gα16 agonist, was used. As depicted in FIG. 13, the expression level of Gα16/Gαgust43 was higher in “Gα-T1R3+T1R2”, and was cap-dependent>IRES-dependent, as in the case of the bitter taste receptor (T2R).

Taken together with the results of Example 9(2), it was demonstrated that the Gα-T1R3 of the present invention causes the high-level expression of both T1R3 and Gα16/Gαgust43.

(4) Response to T1R3 Agonist

“Gα-T1R3+T1R2” and “T1R3-Gα+T1R2” were examined for the response to a T1R3 agonist. Cyclamate (10 mM) was added, and the maximum fluorescence intensity during 40 seconds after addition was obtained. The increase rate of the maximum fluorescence intensity relative to the fluorescence intensity at the start of measurement was calculated as the response intensity of T1R3 to the agonist.

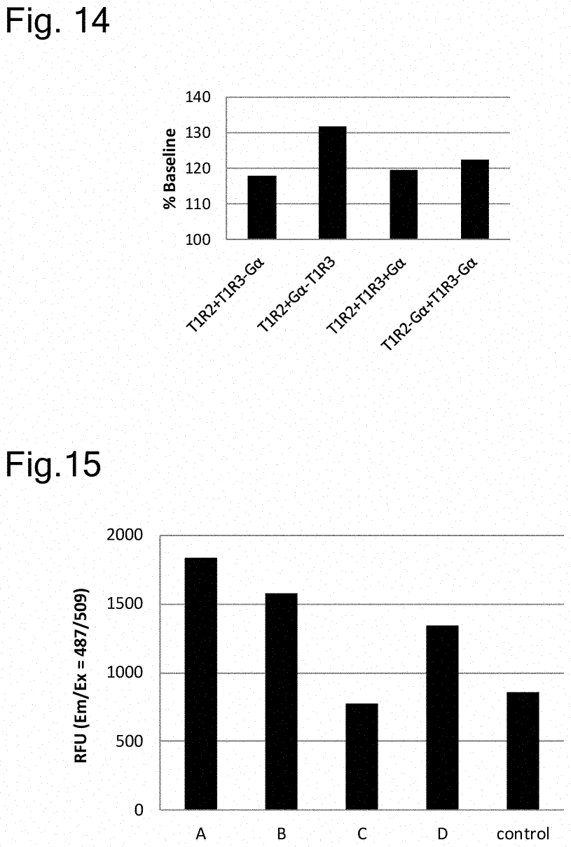

As a comparative example, in addition to “T1R3-Gα+T1R2”, “cells co-transfected with three types of vectors each containing a gene encoding each protein of T1R2, T1R3 and Gα16/Gαgust43”, and “cells co-transfected with T1R2-Gα and T1R3-Gα” were used. As depicted in FIG. 14, “Gα-T1R3+T1R2” exhibited the highest response to the T1R3 agonist.

### Example 10 IRES-Mediated Protein Expression

In order to examine the effect of IRES on the expression level of a protein, proteins other than a GPCR protein were expressed upstream and downstream of IRES. As the protein, a green fluorescent protein (EmGFP) (nucleotide sequence: SEQ ID NO: 78 (GenBank Accession No. EU056363.1); amino acid sequence: SEQ ID NO: 79) was used. Constructs were prepared using pcDNA vectors comprising the following nucleic acid sequences A to D, and then introduced into cell line HEK293T for transient expression thereof.

A: ssr-EmGFP-IRES-Gα

### B: EmGFP-IRES-Gα

### C: Gα-IRES-ssr-EmGFP

### D: Gα-IRES-EmGFP

Control: pcDNA vector containing no inserted gene

Cells were excited at the wavelength of 487 nm and the fluorescence intensity at the wavelength of 509 nm was measured. The fluorescence intensity is proportional to the expression level of EmGFP. The results are depicted in FIG. 15. The expression level of EmGFP protein was higher in cases (A, B) where the EmGFP gene had been placed upstream of IRES, than in cases (C, D) where the same was placed downstream of IRES. The expression level of EmGFP protein was cap-dependent>IRES-dependent as in the case of Gα protein.

### Example 11 T2R16 Expression Level in Cells Transiently Expressing pEAK10-T2R16-Gα and pEAK10-Gα-T2R16

In this example, T2R16 expression level in HEL293T cells transiently expressing pEAK10-IRES-T2R16-Gα and pEAK10-Gα-IRES-T2R16 after gene transfer thereof was examined. Hereinafter, the description of “IRES” may be omitted in Examples 11-15.

A gene sequence encoding T2R16-IRES-Gα and Gα-IRES-T2R16 was subcloned into pEAK10 vectors (Edge Biosystem) to prepare expression vectors pEAK10-T2R16-Gα and pEAK10-Gα-T2R16. The pEAK10 vectors are plasmid vectors each comprising an EF1α promoter and a puromycin resistance gene. The thus prepared expression vectors were introduced into HEK293T cells by the same method as in Example 4 to prepare transiently expressing cells.

The expression level of T2R16 in transiently expressing cells was evaluated by the In cell ELISA using the same technique as in Example 5 (2). As a control, HEK293T cells into which a pEAK expression vector containing no gene inserted therein were used. For detection, as in Example 5 (2), an anti-V5-HRP antibody (Thermo Fisher Scientific Inc.) recognizing the V5 epitope added to the C-terminus was used.

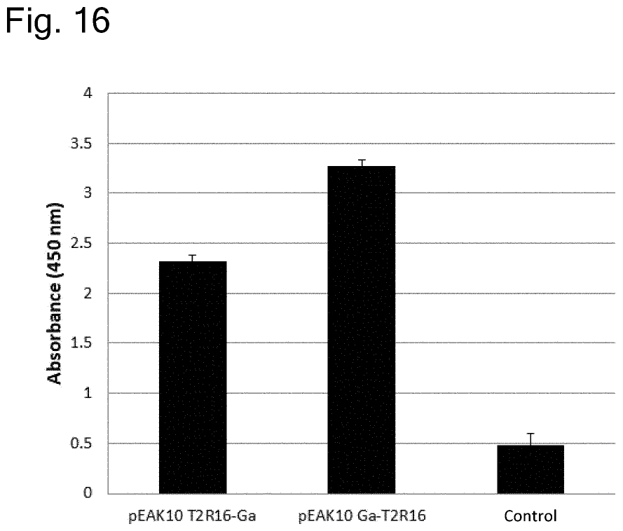

As depicted in FIG. 16, the cells into which the pEAK10-Gα-T2R16 expression vector had been introduced exhibited a higher T2R16 expression level than that exhibited by the cells into which the pEAK10-T2R16-Gα expression vector had been introduced. This demonstrates that the effect of the present invention is not influenced by the gene sequence unique to the vector, such as a promoter and a non-translated region.

### Example 12 Measurement of Response of Transiently Expressing Cells to Bitter Substance (2)

In this example, with respect to seven T2R subtypes (bitter taste receptors), responses of transiently expressing cells to bitter substances were measured.

Seven types of expression vectors comprising each of the genes encoding the seven T2R subtypes listed in Table 5 instead of the gene encoding T2R16 of Gα-T2R16 in Example 4 were prepared, and then the response of transiently expressing cells to known agonists (Table 5) for each subtype was measured by the same method as in Example 4.

TABLE 5

| Subtype | Agonist | Concentration for addition |
|---|---|---|
| T2R4 | Amarogentin | 300 uM |
| T2R8 | Denatonium benzoate | 300 uM |
| T2R9 | Ofloxacin | 100 uM |
| T2R10 | Chloroquine | 3 mM |
| T2R13 | Diphenidole | 100 uM |
| T2R20 | Cromolyn | 1 mM |
| T2R39 | Chloroquinine | 1 mM |

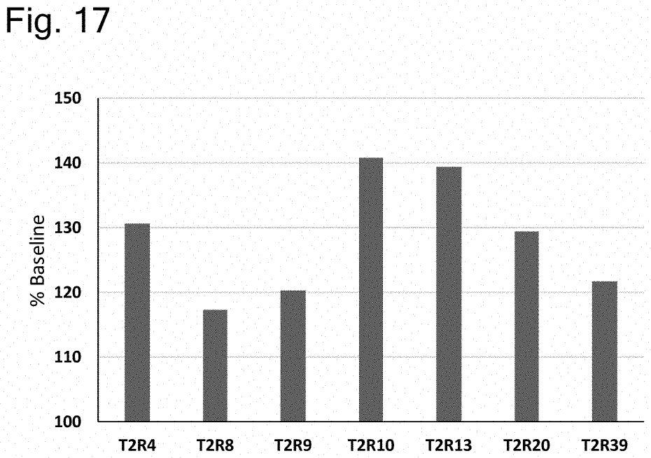

As a result, all T2R subtypes exhibited responses to agonists (FIG. 17).

### Example 13 Measurement of Response of Stably Expressing Cells to Bitter Substance (2)

In this example, with regard to a T2R38 subtype (bitter taste receptor), the response of stably expressing cells to a bitter substance was measured.

A Gα-T2R38 expression vector comprising a gene encoding a T2R38 subtype (bitter taste receptor) instead of the gene encoding T2R16 of Gα-T2R16 in Examples 1 and 2 was prepared, and thus a stable expression cell line was prepared by the same method as in Examples 1 and 2.

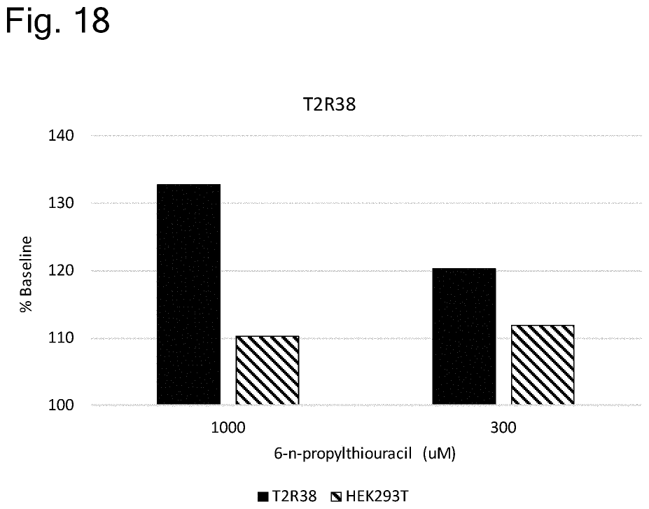

Subsequently, using the same method as in Example 3, the response to a known T2R38 agonist, 6-n-propylthiouracil (1000 μM, 300 μM), was measured. As a control, a cell line (HEK293T) not incorporating any Gα-T2R38 expression vector was used. As a result, responses to 6-n-propylthiouracil (at both 1000 μM and 300 μM) were exhibited (FIG. 18).

### Example 14 Expression Level of Bitter Taste Receptor in Transiently Expressing Cells

In this example, the expression levels of fourteen T2R subtypes (bitter taste receptors) in transiently expressing cells were examined.

Instead of the gene encoding T2R16 of Gα-T2R16 in Example 4, fourteen expression vector types comprising genes encoding each of fourteen T2R subtypes listed in Table 6 were prepared, and thus cells transiently expressing each of T2R and Gα were prepared by the same method as in Example 4.

TABLE 6
T2R Subtype

| | | |
|---|---|---|
| T2R4 | T2R14 | T2R45 |
| T2R1 | T2R19 | T2R46 |
| T2R3 | T2R41 | T2R47 |
| T2R5 | T2R42 | T2R50 |
| T2R7 | T2R44 | T2R60 |

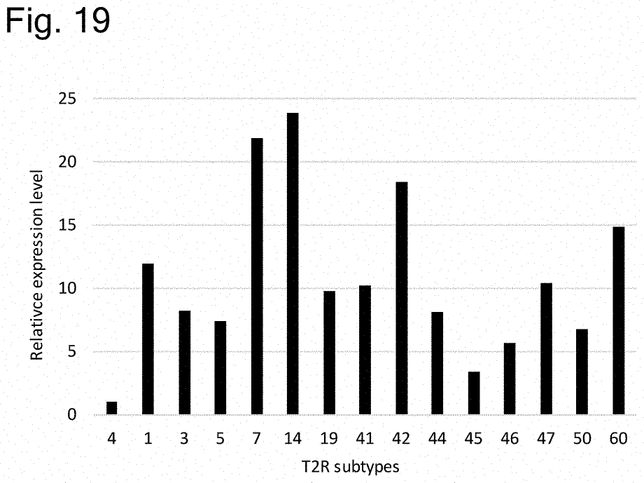

Next, the expression level of T2R in each cell was measured by the In cell ELISA using the same technique as in Example 5 (2). The thus obtained each T2R expression level was compared with the T2R4 expression level in cells transiently expressing Gα-T2R4, the response of which to a known agonist was confirmed in Example 12. As a result, cells exhibited the expression levels of the fourteen T2R subtypes higher than that of T2R4 (FIG. 19). These results demonstrated that the expression levels of the 14 types of T2R were all sufficient for measurement of the response.

### Example 15 Measurement of Response and Expression Level in Cells Transiently Expressing Olfactory Receptor

In this example, the measurement of the response and the expression level in cells transiently expressing an olfactory receptor (OR3A1) were examined.

Genes encoding Rho-OR3A1, in which N-terminal 21 amino acids (Rho) of human rhodopsin had been added to the N-terminus of OR3A1, and Gαolf were introduced together with IRES into pcDNA vectors by the same method as in Example 1, thereby preparing expression vectors, OR3A1-Gαolf and Gαolf-OR3A1. Using these expression vectors, transiently expressing HEK293T cells were prepared by the same method as in Example 4. As a control, HEK293T cells prepared by separate introduction of each gene of OR3A1 and Gαolf into pcDNA and then double-gene transfer thereof were used.

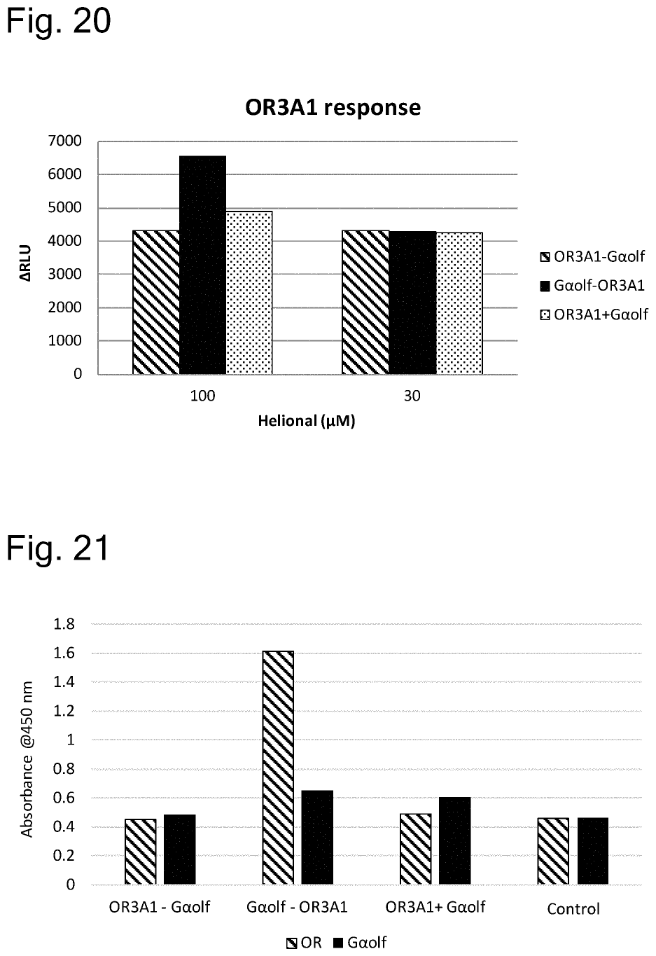

Next, helional, a known agonist for OR3A1, was added onto the prepared cells (100 μM, 30 μM). The amount of cAMP produced in association with the response of OR3A1 20 minutes after the addition of helional was detected by luminescence measurement using a cAMP-GloMax assay kit (Promega). As a result, cells into which the Gαolf-OR3A1 expression vector had been introduced exhibited an increase in cAMP after the addition of 100 μM helional. In the case of introducing the OR3A1-Gαolf expression vector and in the case of separately introducing the OR3A1 and Gαolf genes, no response (increase in cAMP) was observed due to the addition of helional (FIG. 20).

Furthermore, the expression levels of Rho-OR3A1 and Gαolf were compared by In Cell ELISA using an anti-Rho antibody (Merck) and an anti-Gαolf antibody (SANTA CRUZ). As a control, HEK293T cells into which pcDNA containing no such gene inserted therein had been introduced were used. As a result, cells into which Gαolf-OR3A1 had been introduced exhibited higher expression levels of Gαolf and OR3A1 compared to cells into which OR3A1-Gαolf had been introduced (FIG. 21).

1. A construct for expressing a G protein-coupled receptor protein or a subunit thereof, comprising: a nucleic acid encoding a G protein α subunit protein; a nucleic acid comprising an IRES sequence; and a nucleic acid encoding the G protein-coupled receptor protein or a subunit thereof in the stated order from the 5′ side in a single vector.

2. The construct according to claim 1, wherein the G protein-coupled receptor or a subunit thereof is a G protein-coupled receptor of a rhodopsin-like receptor class or a metabotropic glutamate receptor/metabotropic pheromone receptor class.

3. The construct according to claim 1, wherein the G protein-coupled receptor is a G protein-coupled receptor of the rhodopsin-like receptor class.

4. The construct according to claim 1, wherein the G protein-coupled receptor is a bitter taste receptor.

5. The construct according to claim 1, wherein the vector is a plasmid vector.

6. The construct according to claim 1, wherein the vector is a plasmid vector comprising a CMV promoter.

7. The construct according to claim 1, wherein the vector is a plasmid vector comprising a CMV promoter and a hygromycin resistance gene.

8. The construct according to claim 1, wherein the vector is a plasmid vector comprising an EF1α promoter.

9. The construct according to claim 1, wherein the vector is a plasmid vector comprising an EF1α promoter and a puromycin resistance gene.

10. The construct according to claim 1, wherein the IRES sequence is a type 2 IRES sequence.

11. The construct according to claim 1, wherein the IRES sequence is an IRES sequence derived from encephalomyocarditis virus.

12. The construct according to claim 1, comprising a nucleic acid encoding a polypeptide having a function of enhancing the translocation of the G protein-coupled receptor protein or a subunit thereof to a cell membrane, 5′ to the nucleic acid encoding the G protein-coupled receptor protein or a subunit thereof.

13. The construct according to claim 12, wherein the polypeptide having a function of enhancing the translocation of the G protein-coupled receptor protein or a subunit thereof to a cell membrane is a polypeptide comprising N-terminal 45 amino acids of a rat somatostatin receptor or a polypeptide comprising N-terminal 39 amino acids of bovine rhodopsin.

14. The construct according to claim 1, wherein the G protein α subunit protein is a G protein α subunit protein of the Gq family, a G protein α subunit protein of the Gi family, a G protein α subunit protein of the Gs family, or a chimeric protein thereof.

15. The construct according to claim 1, wherein the G protein α subunit protein is Gα16, Gαgust or Gαolf, or a chimeric protein thereof.

16. The construct according to claim 1, wherein the G protein α subunit protein is human Gα16/rat Gαgust.

17. The construct according to claim 16, wherein the G protein α subunit protein is human Gα16/rat Gαgust43.

18. The construct according to claim 1, wherein the G protein-coupled receptor protein is a human G protein-coupled receptor protein.

19. Transformed cells, into which the construct according to claim 1 is introduced.

20. A method for measuring a physiological response to a substance, comprising adding the substance to the transformed cells according to claim 19, and then measuring a physiological response induced by the substance.

21. The measurement method according to claim 20, wherein the substance is a bitter substance, an odorant, an umami substance or a sweet substance, or a modulator for these substances.

22. Use of the transformed cells according to claim 19, for measurement of a physiological response to a substance.

23. A kit comprising the transformed cells according to claim 19, which is used for a method for measuring a physiological response to a substance by adding the substance to the transformed cells and then measuring a physiological response induced by the substance.
